(19) United States
(12) Patent Application Publication
(10) Pub. No.: US 2014/0017787 A1
(43) Pub. Date: Jan. 16, 2014

(54) MESENCHYMAL STEM CELLS AND RELATED THERAPIES

(71) Applicant: Aline M. Betancourt, New Orleans, LA (US)

(72) Inventor: Aline M. Betancourt, New Orleans, LA (US)

(21) Appl. No.: 13/878,734

(22) PCT Filed: Oct. 11, 2011
(86) PCT No.: PCT/US11/55831

§ 371 Date: Jul. 15, 2013

Related U.S. Application Data
(60) Provisional application No. 61/391,749, filed on Oct. 11, 2010.

(51) Int. Cl. C12N 5/0775

(52) U.S. Cl. CPC C12N 5/0675
USPC 435/375

(57) ABSTRACT

Mesenchymal stem cells that selectively promote or suppress inflammation are provided, as well as methods of producing and using the same.

### BACKGROUND

1. Field

The present disclosure relates to mesenchymal stem cells (MSCs), and methods for producing and using the same. More particularly, the present disclosure relates to MSCs that either promote or suppress inflammation, as well as methods of producing and using the same.

2. Description of Related Art

Many human diseases are caused or exacerbated by inappropriate inflammation that is refractory to most current treatment protocols. Nevertheless, sales of products targeting two therapeutic areas (oncology, and arthritis, immune and inflammatory diseases (referred to collectively as “AIID”)) account for approximately one-half of total top-20 protein sales in 2010. The AIID market encompasses a wide-reaching therapeutic area covering a vast array of disease categories. Primary indications include, for example, rheumatoid arthritis (RA), psoriasis, psoriatic arthritis, systemic lupus erythematosis (SLE), Crohn's disease, ulcerative colitis and ankylosing spondylitis.

Oncologic and arthritis, immune and inflammatory diseases represent a significant and recurrent health issue. For example, in 2002 alone, an estimated 43 million adults in the U.S. displayed some form of arthritis, RA, gout, lupus, or fibromyalgia. Arthritis is the leading cause of disability in America, affecting an estimated 45 million people per year. As a cause of disability, inflammation affects more people than back pain, heart or lung conditions, diabetes, or cancer. According to the Arthritis Foundation, inflammation is responsible for 427 million days of restricted activity, 156 million days in bed, and 45 million days lost from work each year. The economic impact is dramatic, and costs the U.S. economy at least $128 billion per year in medical care and lost wages.

Recently, stem cell-based therapies for such diseases have received significant attention. However, current methods for adult stem cell therapy utilize whole stem cell populations that may or may not behave in the manner intended by a physician; the problem is an inability to predict what behavior the infused mix of cells will have in the patient. For example, because mixed and undefined cell populations are infused into patients by current methodologies, there is the potential that some patients receive a population of cells that have undergone differentiation cues and are only capable to fill in where bone cells are needed while another patient may be infused with a mix of cells that solely direct anti-inflammatory behaviors or fat deposition. Thus, the problem is an inability to predict what behavior the infused mix of cells will have in the patient. As such, current stem cell therapies are used blindly.

Solutions to these technical problems are provided by the embodiments characterized in the claims below.

### BRIEF SUMMARY

In one embodiment, an isolated, stimulated mesenchymal stem cell is provided, wherein the stimulated mesenchymal stem cell demonstrates, versus a mesenchymal cell that is not stimulated: elevated secretion of IL4, IL6, and IL8, reduced secretion of TGFβ1, and increased expression of Jagged 1, MIR155, and Bic; or elevated secretion of IL4, IP10, RANTES, IL1RA, PGE2, and SMAD7, reduced expression of TGFβ1, TGFβ3, Jagged 1, MIR155, and Bic, and increased indoleamine 2,3-dioxygenase activity.

In one embodiment, a method for stimulating mesenchymal stem cells is provided, comprising: (a) isolating mesenchymal stem cells into a culture medium; (b) incubating the mesenchymal stem cells of (a) for up to 1 hour with a Toll-like receptor ligand selected from the group consisting of poly (I:C) and lipopolysaccharide; (c) removing said Toll-like receptor ligand from the mesenchymal stem cells of (b); (d) and optionally further incubating the mesenchymal stem cells of (c), thereby stimulating said mesenchymal stem cells. In one aspect of the method, the Toll-like receptor ligand is poly (I:C) at a concentration of about 1 μg/mL of culture medium. In one aspect of the method, the Toll-like receptor ligand is lipopolysaccharide at a concentration of about 10 ng/mL of culture medium.

In one embodiment, an isolated, stimulated mesenchymal stem cell is provided, produced by a process comprising: (a) isolating a mesenchymal stem cell into a culture medium; (b) incubating the mesenchymal stem cell of (a) for up to 1 hour with a Toll-like receptor ligand selected from the group consisting of poly (I:C) and lipopolysaccharide; (c) removing said Toll-like receptor ligand from the mesenchymal stem cell of (b); and (d) optionally further incubating the mesenchymal stem cell of (c), thereby producing said stimulated mesenchymal stem cell.

In one embodiment, an isolated mesenchymal stem cell stimulated with at least one TLR3 ligand is provided, wherein the stimulated mesenchymal stem cell exhibits elevated secretion of IL4, CXCL10 (IP10), CXCL5 (RANTES), PGE2, and IL10, reduced expression of TGFβ1, TGFβ3, Jagged 1, MIR155, and Bic, and increased indoleamine 2,3-dioxygenase activity, in comparison to a mesenchymal cell that is not stimulated with the at least one TLR3 ligand. In an embodiment, the isolated mesenchymal stem cell stimulated with at least one TLR3 ligand further exhibits elevated secretion of IL1RA. In yet another embodiment, the isolated mesenchymal stem cell stimulated with at least one TLR3 ligand further exhibits elevated secretion of SMAD7.

In one embodiment, an isolated mesenchymal stem cell stimulated with at least one TLR4 ligand is provided, wherein the stimulated mesenchymal stem cell exhibits elevated secretion of IL6 and IL8, reduced secretion of TGFβ1, and increased expression of Jagged 1, MIR155, and Bic, in comparison to a mesenchymal cell that is not stimulated with the at least one TLR4 ligand.

In one embodiment, an isolated, stimulated, co-cultured mesenchymal stem cell is provided, wherein the cell is produced by a process comprising (a) isolating a mesenchymal stem cell into a culture medium; (b) incubating the isolated mesenchymal stem cell produced from step (a) for up to 1 hour with (i) a TLR 3 ligand selected from the group consisting of IL4, IL13, poly(A:U), poly(I:C), and combinations thereof, or (ii) a TLR 4 ligand selected from the group consisting of aminoalkyl glucosaminide 4-phosphates, interferons, TNF-alpha, GM-CSF, lipopolysaccharide (LPS), and combinations thereof; (c) isolating human fibroblast-like synoviocyte (FLS) cells derived from rheumatoid arthritis or osteoarthritis into a culture medium comprising TNF-alpha or lipopolysaccharide (LPS); and (d) incubating the isolated mesenchymal stem cell produced from step (b) with the FLS cells produced from step (c); thereby producing said isolated, stimulated, co-cultured mesenchymal stem cell.

In one embodiment, a method of treating ovarian cancer is provided wherein the method comprises delivering an isolated mesenchymal stem cell that is incubated with at least one TLR4 ligand for up to 2 hours.

In one embodiment, an isolated mesenchymal stem cell for use in treating ovarian cancer is provided. The method comprises delivering an isolated mesenchymal stem cell incubated with at least one TLR4 ligand selected from the group consisting of aminoalkyl glucosaminide 4-phosphates, interferons, TNF-alpha, GM-CSF, lipopolysaccharide (LPS), and combinations thereof for up to 2 hours.

In one embodiment, a method of treating diabetic peripheral neuropathy is provided wherein the method comprises delivering an isolated mesenchymal stem cell that is incubated with at least one TLR3 ligand for up to 2 hours.

In one embodiment, an isolated mesenchymal stem cell for use in treating diabetic peripheral neuropathy is provided. The method comprises delivering an isolated mesenchymal stem cell incubated with at least one TLR3 ligand selected from the group consisting of IL4, IL13, poly(A:U), poly(I:C), and combinations thereof for up to 2 hours.

In one embodiment, a method of treating acute lung injury is provided wherein the method comprises delivering an isolated mesenchymal stem cell that is incubated with at least one TLR3 ligand for up to 2 hours.

In one embodiment, an isolated mesenchymal stem cell for use in treating acute lung injury is provided. The method comprises delivering an isolated mesenchymal stem cell incubated with at least one Toll-like receptor 3 ligand selected from the group consisting of IL4, IL13, poly(A:U), poly(I:C), and combinations thereof for up to 2 hours.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows that MSC1 differ from MSC2 in their secretion of immune modulators. The data of FIGS. 1A & 1B show increased expression of known immune suppressive factors by TLR3-primed hMSCs (MSC2) but not by TLR4-primed hMSCs (MSC1). The data of FIGS. 1C & 1D implicate direct TLR3 induction of IP10 (CCL10) and RANTES (CCL5) secretion.

FIG. 2 shows that short-term TLR stimulation promotes the migration of hMSCs.

FIG. 3 shows that TLR3 activation inhibits bone and fat differentiation.

FIG. 4 shows that TLR3-primed hMSCs deposit more fibronectin, while TLR4-primed hMSCs deposit more collagen. FIG. 4A shows the results of immunostaining for collagen I/II and fibronectin, demonstrating that TLR4-primed cells deposit twice as much collagen I/II and half as much fibronectin as TLR3-primed cells. FIG. 4B shows the results of densitometric analysis of the photomicrographs of FIG. 4A, normalized to background absorbance.

FIG. 5 shows that transforming growth factor β (TGFβ1 and 3) expression is diminished in TLR3-primed MSCs compared with measured levels for TLR4-primed and unprimed MSCs. TGFβ2 levels are small but are further repressed by both treatments.

FIG. 6 shows SMAD3 expression and activation (phosphoSMAD3, pSMAD3), as well as SMAD7 expression in hMSCs. The data of FIG. 6A show that SMAD3 is activated in TLR4-primed (increased nuclear pSMAD3) but not TLR3-primed hMSCs. Arrows in FIG. 6A point to corresponding magnified cell nuclei. The data of FIG. 6B show that SMAD7 expression is induced after TLR3 but not TLR4 stimulation of hMSCs.

FIG. 7 shows Jagged 1 and Jagged 2 expression in hMSCs. The data of FIG. 7A show that Jagged 1 expression is elevated, perinuclear, and focused on edges in TLR4-primed but not TLR3-primed hMSCs. Arrows in FIG. 7A point to corresponding magnified cell nuclei. The data of FIG. 7B show that Jagged 2 expression is diffuse in TLR3-primed hMSCs, increased, and perinuclear and endosomal after TLR4 stimulation of hMSCs.

FIG. 8 shows that MSC1 differ from MSC2 in their expression of inflammatory mediators. FIG. 8A shows increased expression of known immune suppressive effector indoleamine 2,3-dioxygenase (IDO); and FIG. 8B shows increased expression of known immune suppressive effector prostaglandin E2 (PGE₂).

FIG. 9 shows that MSC1 support PBMC (T cell) activation, while unprimed MSCs and MSC2 suppress it. FIGS. 9A-9C show differences (arrows) in T cell activation when allogeneic PBMCs are stimulated (PBMCs*) and co-cultured with either untreated MSCs (FIG. 9A), MSC1 (FIG. 9B), or MSC2 (FIG. 9C). FIG. 9D shows expression of Jagged 1 and SMAD7 among the CD45+ non-adherent hPBMCs collected at the end of the MLMR experiments. FIG. 9E shows expression of Jagged 1 and SMAD7 among the CD90+ adherent hMSCs collected at the end of the MLMR experiments.

FIG. 10 shows the effects of human mesenchymal stem cell (MSC)-based therapies on lung integrity in mice with established acute lung injury. FIG. 10A shows total myeloperoxidase (MPO) activity (pmol/mL); FIG. 10B shows BALF total cells (×10³); and FIG. 10C shows BALF protein concentration (μg/mL).

FIG. 11 shows that MSC1 do not support tumor growth, while MSC2 favor tumor growth. FIG. 11A shows the results from donor 1179; and FIG. 11B shows the results from donor 1429. The data of FIGS. 11A and 11B are also provided in tabular form at TABLES 4 and 5, respectively.

FIG. 12 shows that members of the pro-inflammatory microRNA155 family (miRNA 155 and Bic) are elevated in MSC1, and repressed in MSC2.

FIG. 13 shows that human MSC1 based therapy does not support tumor growth, while human MSC2 based therapy favors tumor growth and metastases after treatment of mice with established ovarian tumors. FIG. 13A shows differences in tumor volume of primary tumor growth. FIG. 13B shows differences in relative tumor-associated CD45+ leukocytes. FIG. 13C shows differences in relative tumor-associated F4/80+ macrophage recruitment.

FIG. 14 shows divergent effect of MSC1 and MSC2 on co-cultures of various human cancer cell lines with MSC1 and MSC2. FIG. 14A shows distinct effect in Colony Forming Unit (CFU) assays. FIG. 14B shows distinct effects in 3-D tumor spheroid assays.

FIG. 15 shows effect on heat hyperalgesia of MSC-treatments of streptozotocin (STZ)-induced diabetic mice. Data demonstrate that treatment with MSC2 improves diabetic peripheral neuropathy (DPN) heat hyperalgesia over baseline controls, MSCs, and MSC1.

FIG. 16 shows effect on mechanical allodynia of MSC-treatments of streptozotocin (STZ)-induced diabetic mice. Data demonstrate that treatment with MSC2 improves diabetic peripheral neuropathy (DPN) mechanical allodynia over baseline controls, MSC and MSC1.

FIG. 17 shows cytokine/chemokine secretion in the serum of MSC-treated streptozotocin (STZ)-induced diabetic mice. Data in FIG. 17A show that the serum from mice treated with MSC2 had lower levels of the pro-inflammatory cytokines IL-17 and IL-1 alpha (interleukin). Data in FIG. 17B show that the serum from mice treated with MSC2 had lower levels of the pro-inflammatory cytokines IL-1 beta and IL-2 than the serum from the mice of the other three treatments. Data in FIG. 17C show that the serum from mice treated with MSC2 had lower levels of the pro-inflammatory cytokine IL-6 than the serum from the mice of the other three treatments.

FIG. 18 shows cytokine/chemokine secretion of allogenic co-cultures of human fibroblast-like synoviocytes (FLS) derived from rheumatoid arthritis (RA) patients with varying MSC. FIG. 18A is a graph of TNF-alpha secretion in pg/mL. FIG. 18B is a graph of IL-6 secretion in pg/mL. FIG. 18C is a graph of IFN-gamma in pg/mL. FIG. 18D is a graph of IL-8 secretion in pg/mL. FIG. 18E is a graph of CCL5 secretion in pg/mL. FIG. 18F is a graph of CCL10 secretion in pg/mL. FIG. 18G is a graph of IL-10 secretion in pg/mL. FIG. 18H is a graph of VEGF secretion in pg/mL.

FIG. 19 shows cytokine/chemokine secretion of allogenic co-cultures of human fibroblast-like synoviocytes (FLS) derived from osteoarthritis (OA) patients with varying MSC. FIG. 19A is a graph of TNF-alpha secretion in pg/mL. FIG. 19B is a graph of IL-6 secretion in pg/mL. FIG. 19C is a graph of IFN-gamma secretion in pg/mL. FIG. 19D is a graph of IL-8 secretion in pg/mL. FIG. 19E is a graph of CCL5 secretion in pg/mL. FIG. 19F is a graph of CCL10 secretion in pg/mL. FIG. 19G is a graph of IL-10 secretion in pg/mL. FIG. 19H is a graph of VEGF secretion in pg/mL.

FIG. 20 shows results from quantitative PCR (qPCR)-RNA expression assays of allogenic co-cultures of human fibroblast-like synoviocytes (FLS) derived from rheumatoid arthritis (RA) patients with varying mesenchymal stem cells as indicated FIG. 20 provides graphs of normalized RNA expression determined by the ΔΔ cumulative threshold method (C(t)) with 18srRNA as internal housekeeping target gene. FIGS. 20A & 20B are graphs of normalized RNA expression using TNF-alpha and MMP2 primers, respectively. FIGS. 20C & 20D are graphs of normalized RNA expression using IL-6 and MMP9 primers, respectively. FIGS. 20E & 20F are graphs of normalized RNA expression using MT-MMP1 and uPA primers.

FIG. 21 shows results from a collagen I migration/invasion assay of allogenic co-cultures of human fibroblast-like synoviocytes (FLS) derived from rheumatoid arthritis (RA) patients of osteoarthritis (OA) patients with varying MSC. FIGS. 21A & 21B show graphs of average cell numbers per viewing field. Data presented are the average count of 3 fields per sample. FIG. 21C is an illustration of migrating and invading cells visualized on an inverted fluorescence microscope (200×, Olympus©).

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

The present application provides: methods of priming stem cells (and mesenchymal stem cells, more particularly) to behave in either a pro-inflammatory or an anti-inflammatory manner; the stem cells produced thereby; and methods of using said stem cells. The methods of priming the stem cells ensure that the cells act in a predictable manner to control (either promote or inhibit) inflammation. Mesenchymal stem cells are also referred to by others as “marrow stromal cells” and “multipotent stromal cells”. The additional step of priming the cells to behave in a predictable manner (e.g., in either a pro-inflammatory or an anti-inflammatory manner) dramatically extends the usefulness of mesenchymal stem cell therapy; rendering mesenchymal stem cell behavior predictable enables targeted treatment regimens.

The pro-inflammatory stem cells, methods of producing them, and methods of using them are referred to collectively as “START-IT”, while the anti-inflammatory stem cells, methods of producing them, and methods of using them are referred to collectively as “STOP-IT”.

The products and methods of the present disclosure can be used to produce populations of stem cells that act in a predictable manner to either enhance or inhibit inflammation. They also can be used to treat acute or chronic conditions. For example, pro-inflammatory START-IT cells could be used to treat cancer and other pathogenic infections. Anti-inflammatory STOP-IT cells could be used to treat inflammatory diseases such as inflammatory bowel disease (IBD), Crohn's disease, acute lung injury, chronic pain (e.g., associated with rheumatoid arthritis), and pathogenic infections.

The methods and products of the instant disclosure may be applicable to any type of cancer. One theory underlying the spread of cancer in an organism is evasion of the body's innate immune response. Enhancing the immune response with the START-IT methods and cells can be useful in every type of cancer treatment.

The applicant has indentified a wide range of other conditions (and diseases associated with the conditions) and applications to which the methods and products disclosed may be useful, as shown below:

Anti-aging; cancers (e.g., without limitation, pancreatic, lung, and all other type of cancer); auto-immune diseases (e.g., without limitation, diabetes type 1 (juvenile), diabetes type II, systemic lupus erythematosis (SLE), Sjögren's syndrome, myasthenia gravis, autoimmune cytopenias, scleromyxedema, Crohn's disease, Behcet's disease, rheumatoid arthritis (RA), juvenile arthritis, multiple sclerosis (MS), polychrondritis, systemic vasculitis, alopecia universalis, and Buerger's disease); cardiovascular applications (e.g., without limitation, acute heart damage, coronary artery disease, and myocardial infarction); muscular dystrophies (e.g., without limitation, Duchenne muscular dystrophy, and Becker's muscular dystrophy); disorders of the eye and adnexa (e.g., without limitation, retinitis pigmentosa (RP), keratitis, corneal neovascularization, scleritis, iritis, uveitis, macular degeneration, and glaucoma); immunodeficiencies (e.g., without limitation, severe combined immunodeficiency syndrome, X-linked lymphoproliferative syndrome, X-linked hyper IgM syndrome (XHIGM)); neural degenerative diseases and injuries, (e.g., without limitation, Alzheimer's disease (AD), Parkinson's disease (PD), spinal cord injury, stroke, glaucoma and other neuronal ocular diseases, transient ischemic attack (TIA), amyotrophic lateral sclerosis (ALS)); anemia and other blood conditions, (e.g., without limitation, sickle cell anemia, sideroblastic anemia, aplastic anemia, red cell aplasia, amegakaryocytic thrombocytopenia, thalassemias, primary amyloidosis, diamond blackfan anemia, Fanconi anemia (FA), and chronic Epstein-Barr infection); various wounds and injuries, (e.g., without limitation, limb gangrene, surface wounds, surgical wounds, and acute respiratory distress syndrome (ARDS)); other metabolic disorders, (e.g., without limitation, Hurler syndrome (and other mucopolysaccharidoses), osteogenesis imperfecta, Krabbe disease, osteopetrosis, and cerebral X-linked adrenoleukodystrophy); liver disease, (e.g., without limitation, chronic liver failure, and liver cirrhosis); and bladder disease, (e.g., without limitation, end-stage bladder disease).

Exemplary conditions in which human MSC based therapies can be applied as effective treatment include acute lung injury, neuropathic pain, xenograft related disorders, ovarian cancer, and epithelial ovarian carcinoma. Table A lists data presented herein from studies of various murine disease models with MSCs, MSC1, and MSC2 based therapies. In Table A, MSCs refer to conventionally prepared human MSCs; MSC1 refers to hMSCs incubated for 1 hour with 10 ng/mL LPS and washed prior to delivery; and MSC2 refers to hMSCs incubated for 1 hour with 1 μg/mL poly(I:C) and washed prior to delivery.

TABLE A
Human MSC-based Therapies of Exemplary-Murine Disease Models

| | | | Treatment | | | |
|---|---|---|---|---|---|---|
| | MSC- | MSC | Frequency | | | |
| | based | Dose | (Time of | Disease | Length of | Adverse |
| Animal Disease Model | Therapy | (cells) | treatment) | Impact | study | Effects |
| 1. LPS-induced Acute | MSCs | 0.5 × 10⁶ | 1× 24 hrs | Mostly anti- | 1 week | NONE |
| Lung Injury (ALI) | | | post-disease | inflammatory | post- | |
| (BalbC and C57BL/6J, | MSC1 | 0.5 × 10⁶ | onset) | Pro- | treatment | NONE |
| n = 12) | | | | inflammatory | | |
| | MSC2 | 0.5 × 10⁶ | | Anti- | | NONE |
| | | | | inflammatory | | |
| 2. Streptozotocin- | MSCs | 1-3 × 10⁶ | 3× given in | Mostly anti- | 70 days | NONE |
| Induced Diabetes and | | | 10-day | inflammatory | post- | |
| neuropathic pain | MSC1 | 1-3 × 10⁶ | intervals post- | Pro- | treatment | NONE |
| (C57BL/6J, n = 30) | | | disease onset) | inflammatory | | |
| | MSC2 | 1-3 × 10⁶ | | Anti- | | NONE |
| | | | | inflammatory | | |
| 3. Immune-incompetent | MSCs | 0.5 × 10⁶ | 3× given | Mostly anti- | >120 days | NONE |
| human tumor xenografts | | | weekly post- | inflammatory | post- | |
| (Balb scid and nude n = 60) | MSC1 | 0.5 × 10⁶ | disease onset) | Pro- | treatment | NONE |
| | | | | inflammatory | | |
| | MSC2 | 0.5 × 10⁶ | | Anti- | | NONE |
| | | | | inflammatory | | |
| 4. Immune-competent | MSCs | 0.5 × 10⁶ | 3× given | Mostly anti- | >70 days | NONE |
| MOSEC (C67/BL6J | | | weekly post- | inflammatory | post- | |
| n = 20) | | | disease onset) | | treatment | |

Both STOP-IT and START-IT are disruptive technologies that are indicated for treatment of arthritis, immune diseases, and inflammatory diseases. STOP-IT and START-IT cells and methods could have applications in line with the most studied stem cell fields, for example, as catalysts for heart disease healing, as cancer markers by increasing a host's immune response to carcinogenic cells, for suppressing immunity after transplants, or any other application where magnifying or suppressing the immune response is desired.

The present disclosure provides (among other things) new stem cell-based therapies that allow on-site repair of the aberrant inflammation in a manner that has not been possible previously. The applicant has designed approaches for one therapy that exclusively restores repressed inflammation (termed START-IT) and another that quells over-active inflammation (termed STOP-IT). As disclosed herein, priming of human mesenchymal stem cells (MSCs) with specific Toll-like receptor (TLR) agonists programs (or “polarizes”) them into two different but homogenously acting phenotypes (MSC1 and MSC2) that are exploited individually in START-IT and STOP-IT protocols, respectively. Surprisingly, stimulation of TLR4 within MSCs results in the elevated secretion of pro-inflammatory mediators, while stimulation of TIR3 of MSCs leads to the secretion of factors with mostly immunosuppressive properties. This concept is also based on the reported clinical experience that infused unprimed MSCs are well tolerated even in allogeneic hosts, that they naturally respond and track to injured tissues, and finally, that their established clinical benefit is mostly through local immune modulation. For example, an MSC-based therapy is being marketed by Osiris Therapeutics, which has fast-track FDA approval for the use of these cells. Osiris has already reported some success in allogeneic MSC cell-based therapy clinical trials in graft-versus host disease, type 1 diabetes mellitus, chronic obstructive pulmonary disease (COPD), and myocardial infarction. The instant disclosure improves on MSC-based therapy by priming heterogeneous preparations of MSCs into defined phenotypes (pro- and anti-inflammatory).

As used herein, the term “pro-inflammatory” and “pro-inflammation” refers to any state or condition characterized by an increase of at least one indication of localized inflammation (such as, but not limited to, heat, pain, swelling, and redness) and/or a change in systemic state characterized by (i) an increase of at least one pro-inflammatory immune cell (such as, but not limited to, neutrophils, B-lymphocytes, T-lymphocytes (such as, but not limited to, T-helper cell-1(Th1) and -17(Th17) cells), macrophages, natural killer (NK) cells, and/or mast cells), pro-inflammatory cytokine such as, but not limited to, interleukin-1 (IL-1) and tumor necrosis factor (TNF), or pro-inflammatory chemokine; and (ii) a decrease of at least one anti-inflammatory immune cell (such as, but not limited to, T-helper cell-2 (Th2), T-lymphocyte regulatory cells (Tregs), and/or macrophages), anti-inflammatory cytokine, or anti-inflammatory chemokine. A representative listing of pro-inflammatory and anti-inflammatory cytokines and chemokines is provided, for example, in Dinarello, C., “Proinflammatory Cytokines”, Chest, 118: 503-508 (2000), the disclosure of which is incorporated herein by reference.

As used herein, the term “anti-inflammatory”, “anti-inflammation”, “immunosuppressive”, and “immunosuppressant” refers to any state or condition characterized by a decrease of at least one indication of localized inflammation (such as, but not limited to, heat, pain, swelling, and redness) and/or a change in systemic state characterized by (i) a decrease of at least one pro-inflammatory immune cell, pro-inflammatory cytokine, or pro-inflammatory chemokine; and (ii) an increase of at least one anti-inflammatory immune cell, anti-inflammatory cytokine, or anti-inflammatory chemokine.

The methods disclosed simultaneously transform heterogeneous MSC preparations into uniform MSC preparations, and effectively program the resulting MSCs to repair inflammation as needed and restore the targeted diseased sites. The applicant anticipates that polarized MSC-based therapies will readily treat both acute and chronic inflammatory diseases. Furthermore, as the STOP-IT and START-IT treatments involve cells and not single agents, resistant diseases will not develop after multiple or long-term treatments. Additionally, MSCs are immune-privileged and are not expected (or known) to elicit immune rejection mechanisms following multiple treatments. Finally, manipulation of TLRs is believed to be safe, as several FDA approved biologicals that target or manipulate TLRs have been used for many years without clinical consequence.

The stimulation of specific Toll-like receptors (TLRs) affects the immune modulating responses of human multipotent mesenchymal stromal cells (hMSCs). Toll-like receptors recognize “danger” signals, and their activation leads to profound cellular and systemic responses that mobilize innate and adaptive host immune cells. The danger signals that trigger TLRs are released following most tissue pathologies. Since danger signals recruit immune cells to sites of injury, the applicant reasoned that hMSCs might be recruited in a similar way. Indeed, the present disclosure shows that hMSCs express several TLRs (e.g., TLR3 and TLR4), and that their migration, invasion, and secretion of immune modulating factors is drastically affected by specific TLR-agonist engagement. In particular, applicant noted diverse consequences to the hMSCs following stimulation of TLR3 when compared to TLR4 by a low-level, short-term TLR-priming protocol.

Here, the applicant shows the effect on immune modulation due to priming of specific hMSC TLRs and, based on the findings, presents a new paradigm for therapies using hMSCs. Specifically, hMSCs can be polarized by downstream TLR signaling into two homogenously acting phenotypes, classified here as MSC1 and MSC2. This concept is based partly upon the applicant's observations that TLR4-primed hMSCs (or MSC1), mostly elaborate pro-inflammatory mediators, while TLR3-primed hMSCs (or MSC2), express mostly immunosuppressive ones. Additionally, allogeneic cocultures of TLR-primed MSCs with peripheral blood mononuclear cells (PBMCs) predictably lead to suppressed T-lymphocyte activation following MSC2 co-culture, and permissive T-lymphocyte activation following MSC-1 co-culture.

The present disclosure provides an explanation for some of the conflicting reports on the net effect of TLR stimulation and its downstream consequences on the immune modulating properties of stem cells. The applicant further suggests that MSC polarization provides a convenient way to render these heterogeneous preparations of cells more uniform, and provides an important aspect to consider for the improvement of current stem cell-based therapies.

Multipotent mesenchymal stromal cells (formerly known as mesenchymal stem cells, MSCs) are readily separated from other bone marrow-derived cells by their tendency to adhere to plastic. MSCs differentiate into osteoblasts, chondrocytes, and adipocytes under appropriate culture conditions, as shown by the following references, which are incorporated by reference in their entireties: Abdi R et al. (2008) Immunomodulation by mesenchymal stem cells: a potential therapeutic strategy for type 1 diabetes. Diabetes 57: 1759-1767; Prockop D J (2009) Repair of tissues by adult stem/progenitor cells (MSCs): controversies, myths, and changing paradigms. Mol Ther 17: 939-946; Aggarwal S et al. (2005) Human mesenchymal stem cells modulate allogeneic immune cell responses. Blood 105: 1815-1822; and Phinney D G et al. (1999) Donor variation in the growth properties and osteogenic potential of human marrow stromal cells. J Cell Biochem 75: 424-436. Further, they offer the advantage that they are easily expanded and stored ex vivo and are considered to be “immunoprivileged.” Thus, once harvested, they can safely be infused into either autologous or allogenous hosts owing to their lack of host immune reactivity, as shown by Prockop (2009). Moreover, these cells home to damaged tissues and contribute to their repair by secretion of cytokines, chemokines, and extracellular matrix proteins, as shown by Aggarwal (2005) and by Ryan J M et al. (2005) Mesenchymal stem cells avoid allogeneic rejection. J Inflamm (Lond) 2:8, which is incorporated by reference in its entirety. Because of these qualities, MSCs are very attractive candidates for stem cell-based tissue repair and gene therapy strategies. Numerous investigators have now demonstrated the successful recruitment and multi-organ engraftment capability of infused MSCs in various animal models and human clinical trials, as shown by the following references, which are incorporated by reference in their entireties: Caplan AI (1995) Osteogenesis imperfecta, rehabilitation medicine, fundamental research and mesenchymal stem cells. Connect Tissue Res 31: S9-14; Nakamizo A et al. (2005) Human bone marrow-derived mesenchymal stem cells in the treatment of gliomas. Cancer Res 65: 3307-3318; Aicher A et al. (2003) Assessment of the tissue distribution of transplanted human endothelial progenitor cells by radioactive labeling. Circulation 107: 2134-2139; Picinich S C et al. (2007) The therapeutic potential of mesenchymal stem cells. Cell- & tissue-based therapy. Expert Opin Biol Ther 7: 965-973; and Ozawa K et al. (2008) Cell and gene therapy using mesenchymal stem cells (MSCs). J Autoimmun 30: 121-127. However, the precise molecular mechanisms governing infused stem cell fate, their mobilization, and their recruitment to the sites of engraftment are not fully understood. Additionally, even though a clear clinical benefit is observed when MSCs are used in cell-based therapies, few infused cells (0.1-1%) are found at the target site, as shown by the following references, which are incorporated by reference in their entireties: Prockop (2009); Ohtaki H et al. (2008) Stem/progenitor cells from bone marrow decrease neuronal death in global ischemia by modulation of inflammatory/immune responses. Proc Natl Acad Sci USA 105: 14638-14643; and Gao J et al. (2001) The dynamic in vivo distribution of bone marrow-derived mesenchymal stem cells after infusion. Cells Tissues Organs 169: 12-20. This observation has prompted some to suggest that the benefit observed is due to local immune modulation by these cells rather than to differentiation or replacement of the damaged target tissue by the infused stem cells.

A connection between the stimulation of specific Toll-like receptors (TLRs) and the immune modulating responses of human multipotent mesenchymal stromal cells (hMSCs) was shown by the following references, which are incorporated by reference in their entireties: Hwa Cho H et al. (2006) Role of toll-like receptors on human adipose-derived stromal cells. Stem Cells 24: 2744-2752; Pevsner-Fischer M et al. (2007) Toll-like receptors and their ligands control mesenchymal stem cell functions. Blood 109: 1422-1432; and Tomchuck S L et al. (2008) Toll-like receptors on human mesenchymal stem cells drive their migration and immunomodulating responses. Stem Cells 26: 99-107. Toll-like receptors recognize “danger” signals, and their activation leads to profound cellular and systemic responses that mobilize innate and adaptive host immune cells, as shown by the following references, which are incorporated by reference in their entireties Akira S et al. (2003) Toll-like receptors and their signaling mechanisms. Scand J Infect Dis 35: 555-562; Miggin S M et al. (2006) New insights into the regulation of TLR signaling. J Leukoc Biol 80: 220-226; Takeda K et al. (2003) Toll-like receptors Annu Rev Immunol 21: 335-376; West A P et al. (2006) Recognition and signaling by toll-like receptors. Annu Rev Cell Dev Biol 22: 409-437; and Matzinger P (2002) The danger model: a renewed sense of self. Science 296: 301-305. The TLRs consist of a large family of evolutionarily conserved receptors (e.g., TLR1-9). The danger signals that trigger TLRs are released following most tissue pathologies. Exogenous danger signals typically released after microbial infections are exemplified by endotoxin or lipopolysaccharide (LPS) sheddings. Endogenous danger signals released into the circulation by aberrant or wounded cells are exemplified by intracellular components, such as heat shock proteins or RNA. Typically, these danger signals activate TLRs on sentinel innate immune cells (e.g., dendritic cells), and so initiate an appropriate host response that reestablishes homeostasis, as shown by Akira (2003), Miggin (2006), Takeda (2003), and West (2006). Not wishing to be bound by theory, it is believed that hMSCs might employ TLRs to find tissues in need of their reparative mission, because danger signals recruit immune cells to sites of injury. Surprisingly, not only do hMSCs express several TLRs but the hMSCs' capability to migrate, invade, and secrete immune modulating factors is drastically affected by specific TLR-agonist engagement. In particular, TLR3 stimulation leads to the secretion of factors with mostly immune suppressive properties, while stimulation of TLR4 with LPS results in the secretion of more pro-inflammatory factors.

Other investigations have evaluated the effects of TLR engagement on the typical stromal stem cell properties of tri-lineage differentiation (chondrogenic, osteogenic, adipogenic) and proliferation. For instance, Hwa Cho (2006) described a role for TLRs in proliferation and differentiation of human adipose-derived stem cells (hADSCs). According to Pevsner-Fischer (2007), murine MSCs (muMSCs) were found to express TLRs that upon activation affected their proliferation and differentiation. However, in contrast to hMSCs, Pevsner-Fischer (2007) suggested that activation of TLR2 inhibits both differentiation and migration of muMSCs while simultaneously promoting their proliferation. Liotta (2008) found no effect of TLR activation on adipogenic, osteogenic, or chondrogenic differentiation in hMSCs: Liotta F et al. (2008) Toll-like receptors 3 and 4 are expressed by human bone marrow-derived mesenchymal stem cells and can inhibit their T-cell modulatory activity by impairing Notch signaling. Stem Cells 26: 279-289, which is incorporated by reference in its entirety. Furthermore, and in contrast to the instant disclosure, the report by Liotta (2008) suggested equivalent roles for TLR3 and TLR4 engagement in hMSC immune modulation. Recently, Lombardo (2009) reported that TLR3 and TLR4 engagement within hADSCs increased osteogenic differentiation but had no effect on their adipogenic differentiation or proliferation: Lombardo E et al. (2009) Toll-like receptor-mediated signaling in human adipose-derived stem cells: implications for immunogenicity and immunosuppressive potential. Tissue Eng Part A 15: 1579-1589, which is incorporated by reference in its entirety. Lombardo (2009) also concluded that TLR2, TLR3, and TLR4 activation does not affect the ability of hADSCs to suppress lymphocyte activation, in contrast to the Liotta (2008) report.

The recently described immune modulating properties of hMSCs are rather complex. For instance, immune modulation by hMSCs is attributed to not only secretion of soluble factors but also to direct contact between MSCs and immune cells, as shown by Gur-Wahnon D et al. (2007) Contact-dependent induction of regulatory antigen-presenting cells by human mesenchymal stem cells is mediated via STAT3 signaling. Exp Hematol 35: 426-433, which is incorporated by reference in its entirety. MSCs express low levels of human leukocyte antigen (HLA) major histocompatibility complex (MHC) class I, do not express co-stimulatory molecules (B7-1, -2, CD40, or CD40L), and can be induced to express MHC class II and Fas ligand, which explains why they do not activate alloreactive T cells. MSCs inhibit dendritic cell (DC) maturation, B and T cell proliferation and differentiation, and natural killer (NK) cell activity, and they also support suppressive T regulatory cells (T-regs), as shown by Aggarwal (2005), Gur-Wahnon (2007), and by the following references which are incorporated by reference in their entireties: Nemeth K et al. (2010) Modulation of bone marrow stromal cell functions in infectious diseases by toll-like receptor ligands. J Mol Med 88: 5-10; Nauta A J et al. (2007) Immunomodulatory properties of mesenchymal stromal cells. Blood 110: 3499-3506; and Fibbe W E et al. (2007) Modulation of immune responses by mesenchymal stem cells. Ann N Y Acad Sci 1106: 272-278. Several factors are associated with these immune modulating properties of MSCs, including transforming growth factor beta (TGFβ), hepatocyte growth factor (HGF), HLA-G, prostaglandin (PGE2), IL-10, indoleamine 2,3-dioxygenase (IDO), and interferon-gamma (IFN-γ), as shown by Aggarwal (2005), and by the following references which are incorporated by reference in their entireties: Di Nicola M et al. (2002) Human bone marrow stromal cells suppress T-lymphocyte proliferation induced by cellular or nonspecific mitogenic stimuli. Blood 99: 3838-3843; Meisel R et al. (2004) Human bone marrow stromal cells inhibit allogeneic T-cell responses by indoleamine 2,3-dioxygenase-mediated tryptophan degradation. Blood 103: 4619-4621; Sotiropoulou P A et al. (2006) Interactions between human mesenchymal stem cells and natural killer cells. Stem Cells 24: 74-85; Le Blanc K et al. (2003) Mesenchymal stem cells inhibit and stimulate mixed lymphocyte cultures and mitogenic responses independently of the major histocompatibility complex. Scand Immunol 57: 11-20; Beyth S et al. (2005) Human mesenchymal stem cells alter antigen-presenting cell maturation and induce T cell unresponsiveness. Blood 105: 2214-2219; Krampera M et al. (2006) Role for interferon-gamma in the immunomodulatory activity of human bone marrow mesenchymal stem cells. Stem Cells 24: 386-398; and Rizzo R et al. (2008) A functional role for soluble HLA-G antigens in immune modulation mediated by mesenchymal stromal cells. Cytotherapy 10: 364-375. More recently a role for the notch family member Jagged1 in immune modulation was specifically attributed to downstream TLR signaling of MSCs, as shown by Liotta (2008).

With this in mind, an explanation for the contrasting and complex immune modulating effects reported thus far for TLR activation in most stem cells may come from reinterpretation of all of the data after taking into account the fact that most of the current stem cell preparations yield heterogeneous pools of cells, as well as acknowledging that TLRs expressed on different cell types and from different species (mouse and man) do not always have the same responses (as shown by Heinz S et al. (2003) Species-specific regulation of Toll-like receptor 3 genes in men and mice. J Biol Chem 278: 21502-21509, and Mestas J et al. (2004) Of mice and not men: differences between mouse and human immunology. J Immunol 172: 2731-2738, which are incorporated by reference herein in their entireties). Other important contributing factors that may account for the conflicting reports in the TLR responses of stem cells are the concentration and length of incubation with the specific TLR agonist, along with careful attention to and safeguarding against common LPS (TLR4 agonist) contamination in the laboratory.

The present disclosure extends the studies on TLRs and immune modulation by hMSCs, provides support for these concepts, and builds on the applicant's initial observations that low-level, short-term stimulation with specific TLR3 and TLR4 agonists (or TLR-priming) within hMSCs mediates distinct immune modulating responses. The present disclosure establishes that stimulation of monocytes with interferon-γ- or endotoxin (or LPS, a TLR4-agonist) polarizes them into a classical M1 phenotype that participates in early pro-inflammatory responses, while IL-4 treatment of monocytes yields the alternate M2 phenotype that is associated with later anti-inflammatory resolution responses, as shown by Verreck F A et al. (2006) Phenotypic and functional profiling of human proinflammatory type-1 and anti-inflammatory type-2 macrophages in response to microbial antigens and IFN-gamma- and CD40L-mediated costimulation. J Leukoc Biol 79: 285-293, which is incorporated by reference in its entirety. The present disclosure reveals a new aspect of hMSC biology implied by this work, which suggests that hMSCs, like monocytes, are polarized by downstream TLR signaling into two homogenously acting phenotypes we classify here as MSC1 and MSC2, following the monocyte nomenclature. This disclosure shows that hMSCs respond, following specific TLR priming, in a manner analogous to monocytes which will ultimately help make MSC preparations more uniform, and will be important to study and consider in future improved designs of stem cell-based therapies.

### Materials & Methods

### MSCs

Primary human MSCs (hMSCs) were obtained from the Tulane University Center for Gene Therapy. Additionally, hMSCs were obtained from Lonza (Walkersville, Md.) to ensure variability of the starting cell population, and to ensure that the findings are universal and not unique to single donor pools derived from a unique source. All of the MSC donor preparations from these sources were tested for hematopoietic stem cell markers by the sources and by the applicant. All MSC preparations used were less than 1% positive for CD34 and CD45. To maintain consistency, MSCs of a passage number no greater than 4 were used in all the experiments. Also, no fewer than 5 different unrelated donor MSC pools were tested in all experiments. Throughout these studies, MSCs from unique donors were tested individually and were never pooled with other donors.

### TLR Priming Protocol

In this study, LPS (10 ng/mL, Sigma-Aldrich, St. Louis, Mo.) and polyinosinic:polycytidylic acid (or poly(I:C); 1 μg/mL, Sigma-Aldrich) were used as the agonists for TLR4 and TLR3, respectively. Typically, hMSCs were grown to 60-70% confluency in growth medium (DMEM-alpha and 16.5% fetal calf serum (FCS)) prior to the start of an experiment. TLR-agonists were added to fresh growth medium and incubated with the cells for 1 hr. Then, the cells were washed twice in growth medium without the TLR-agonists and assayed as described for the experiments. Without wishing to be bound by theory, short incubation times (<1 hr) and minimal TLR agonist exposure at the concentrations noted above (or lower) are important for achieving the desired phenotypes and, further, this protocol mimics the gradient of danger signals that endogenous MSCs encounter and respond to at a distance from the site of injury.

The TLR3 agonist may be IL4, IL13, poly(A:U), poly(I:C), and combinations thereof, and may be delivered by incubation, transfection, transduction, by carrier molecules, or by combinations thereof. Preferably, the TLR3 agonist is poly(I:C). The TLR4 agonist may be aminoalkyl glucosaminide 4-phosphates, interferons, TNF-alpha, GM-CSF, lipopolysaccharide (LPS), and combinations thereof, and may be delivered by incubation, transfection, transduction, by carrier molecules, or by combinations thereof. Preferably, the TLR4 agonist is LPS.

The agonist or agonists may be delivered by incubation, transfection, transduction by carrier molecules, or by other techniques known to those of ordinary skill in the art.

The TLR3 agonist may be provided in an amount from about 10 pg/mL to about 100 mg/mL, from about 10 pg/mL to about 10 mg/mL, from about 100 pg/mL to about 1 mg/mL, from about 100 pg/mL to about 100 μg/mL, from about 1 ng/mL to about 100 μg/mL, from about 5 ng/mL to about 100 μg/mL, from about 10 ng/mL to about 100 μg/mL, from about 100 ng/mL to about 100 μg/mL, from about 0.1 μg/mL to about 50 μg/mL, from about 0.1 μg/mL to about 10 μg/mL, from about 0.25 μg/mL to about 7.5 μg/mL, from about 0.5 μg/mL to about 5 μg/mL, from about 1 μg/mL to about 2.5 μg/mL, preferably from about 1 μg/mL to about 1.5 μg/mL, and also preferably about 1 mg/mL in culture medium.

The TLR4 agonist may be provided in an amount from about 10 pg/mL to about 10 μg/mL, from about 100 pg/mL to about 10 μg/mL, from about 1 ng/mL to about 1 μg/mL, from about 5 ng/mL to about 1 μg/mL, from about 10 ng/mL to about 1 μg/mL, from about 100 ng/mL to about 1 μg/mL, preferably from about 5 ng/mL to about 50 ng/mL, preferably from about 5 ng/mL to about 25 ng/mL, and also preferably about 10 ng/mL in culture medium.

The cells may be incubated with TLR agonist for from about 1 minute to about 240 minutes, from about 5 minutes to about 210 minutes, from about 10 minutes to about 180 minutes, from about 15 minutes to about 150 minutes, from about 20 minutes to about 120 minutes, from about 25 minutes to about 90 minutes, from about 30 minutes to about 80 minutes, from about 35 minutes to about 70 minutes, from about 40 minutes to about 65 minutes, from about 45 minutes to about 60 minutes, from about 55 minutes to about 60 minutes, from about 1 minute to about 60 minutes, from about 5 minutes to about 60 minutes, from about 10 minutes to about 60 minutes, from about 15 minutes to about 60 minutes, from about 20 minutes to about 60 minutes, from about 30 minutes to about 60 minutes, and preferably about 60 minutes.

### LPS Contamination

Rigorous testing for LPS contamination was routinely performed on all of the reagents used to avoid spurious conclusions due to this potential TLR-agonist contaminant (Limulus amebocyte lysate chromogenic endpoint assay, Hycult Biotechnologies, The Netherlands). Additionally, all reagents were aliquotted for single or minimal use portions to further prevent contamination.

### TLR3 and TLR4 Inhibition

hMSCs were grown to 70% confluence, harvested, then transfected with pZERO-hTLR3 and pZERO-hTLR4 (Invivogen) using 250 ng plasmid/1×10⁶ cells (nucleofector). 50 ng pMAX-GFP was transfected alone for control, and co-transfected with the pZERO plasmids to monitor transfection efficiency. Each transfection was plated across half of a 24-well plate and allowed to recover for 48 hr. Cells from each transfection were left untreated or were stimulated with TLR3 and TLR4 agonists for 1 hr, washed, and then incubated for 48 hr. Conditioned medium was harvested and stored at −80° C. until analysis. Transfection efficiency was also monitored by co-transfection with 500 ng NF- [symbol] B-promoter driven luciferase (LUC)-expressing plasmid (Stratagene/Agilent Technologies La Jolla, Calif.). Transfection efficiency was determined by these methods to be 30-35% of the cells.

### BioPlex Assays

MSCs were plated at a density of 5×10⁴ in 24-well plates, allowed to adhere overnight, then primed with TLR agonists for 1 hr as indicated. Conditioned medium was collected after 48 hrs and analyzed with Bio-Plex Cytokine Assays (Human Group I & II; Bio-Rad, Hercules, Calif.) following the manufacturer's instructions. These experiments were performed at least three times on three individual MSC donor pools.

### Transwell Migration/Invasion Assay

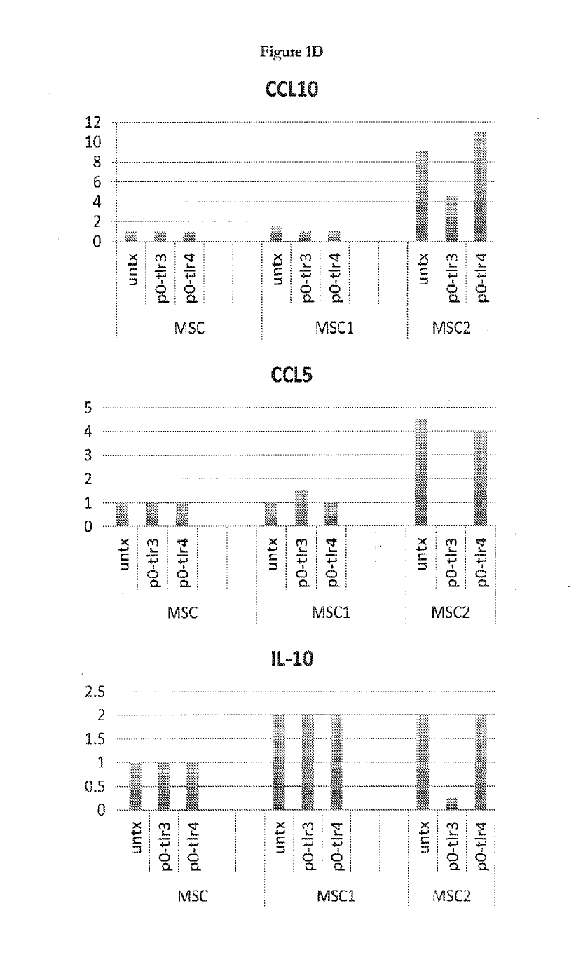

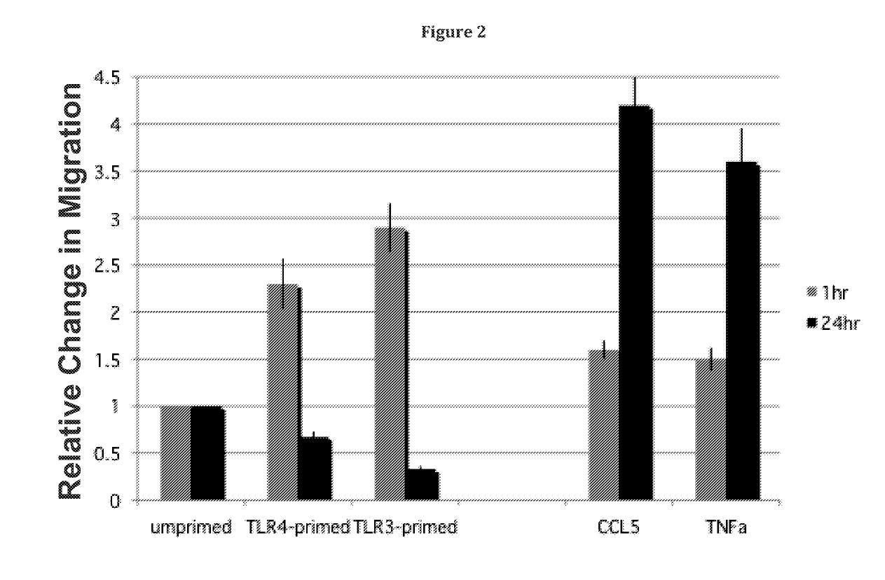

Migration assays were performed in transwell inserts with 8-mm pore membrane filters pre-coated with growth factor-reduced Matrigel™ layer to mimic basement membranes, as described in Tomchuck (2008) and in Zwezdaryk K J et al. (2007) Erythropoietin, a hypoxia-regulated factor, elicits a pro-angiogenic program in human mesenchymal stem cells. Exp Hematol 35: 640-652, both of which are incorporated by reference in their entireties. TLR-primed or unprimed cells were grown to subconfluence (70%) prior to harvesting by trypsinization and labeling with CellTracker™ green (1 μM, Molecular Probes, Eugene, Oreg.) for 1 hr at 37° C. Fluorescently labeled hMSCs (2.5 to 5×10⁵ cells/well in 300 μL) were loaded onto the upper chamber, and 500 μL hMSC growth medium was loaded onto the bottom chamber. After overnight incubation, the upper side of each filter was carefully washed with cold PBS and remaining non-migrating cells were removed with a cotton-tipped applicator. Fluorescence images of the migrating cells were collected using a Nikon TE300 inverted epifluorescence microscope (DP Controller v1.2.1.108, Olympus Optical Company, LTD; Nikon USA, Lewisville, Tex.). Each experiment was performed in triplicate with five separate hMSCs donors. Data are expressed as numbers of counted, migrated cells per 200× field micrograph for each sample well, and normalized to those cell counts obtained for the untreated control (see FIG. 2).

hMSC Tri-lineage Differentiation Protocols

The hMSC tri-lineage differentiation protocols used are described in Pittenger M F et al. (1999) Multilineage potential of adult human mesenchymal stem cells. Science 284: 143-147, which is incorporated by reference herein in its entirety.

### Chondrogenic Differentiation

hMSCs (2.5×10⁵) were placed into defined chondrogenic medium and gently centrifuged (800×g for 5 minutes) in a 15 mL conical tube, where they consolidated into a cell mass or pellet within 24 hours. Chondrogenic medium (CM) consists of high glucose (4.5 g/L) DMEM supplemented with ITS+1 (6.25 μg/mL insulin, 6.2 μg/mL transferrin, 6.25 μg/mL selenous acid, 5.33 μg/mL linoleic acid, 1.25 mg/mL bovine serum albumin (BSA)), 0.1 μM dexamethasone, 10 ng/mL TGFβ3, 50 μg/mL ascorbate 2-phosphate, 2 mM pyruvate, and antibiotics. TGFβ3 is prepared fresh from lyophilized powder, and CM in cultures is replaced every third day. At harvest, the samples are fixed in 10% neutral buffered formalin for several hours, and then processed and embedded in paraffin. Sections of chondrogenic pellets were stained with Safranin O to detect the accumulation of proteoglycans.

### Osteogenic Differentiation

hMSCs are cultured at 3×10⁴ cells/well in 6-well plates in growth medium to 70% confluency, after which the medium is replaced with medium containing osteogenic supplements (OS). OS consists of 50 μM ascorbate 2-phosphate, 10 mM β-glycerol phosphate, and 10⁻⁸M dexamethasone. After three weeks, cells are fixed and stained for 10 minutes with 40 mM Alizarin Red (pH 4.1) to visualize calcium deposition in the ECM.

### Adipogenic Differentiation

Adipogenic induction medium (MDI+I medium): 1 μM dexamethasone and 0.5 mM methyl-isobutylxanthine, 10 μg/mL insulin, 100 μM indomethacin, and 10% FBS in DMEM (4.5 g/L glucose) was added to the confluent layer of hMSCs for 48-72 hr. The medium was then changed to adipogenic maintenance medium for 24 hours. Adipogenic maintenance (AM medium) contained 10 μg/mL insulin and 10% FBS in DMEM (4.5 g/L glucose). The cells were treated twice more with MDI+I, for a total of three treatments. The cells were washed with PBS and fixed in 10% formalin for 1 h at 4° C., stained for 10-15 minutes at room temperature with a working solution of Oil Red O stain, then rinsed 3× with distilled water.

### Flow Cytometry

Human MSCs were harvested and analyzed by flow cytometry with a BD-FACSCalibur flow cytometer (BD Biosciences, San Jose, Calif.) as described previously in Zwezdaryk (2007), incorporated by reference in its entirety. Intracellular antibody staining was achieved after fixation and permeabilization of the cells as indicated by the manufacturer (cytofix/cytoperm buffers, BD Biosciences, San Jose, Calif.). Isotype controls and untreated or unstained samples were run in parallel, as standard. Analysis of MSC donor pools was performed on a BD-FACSCAlibur (BD Biosciences, San Jose, Calif.) using BD CellQuest Pro software. Multi-color flow cytometry analysis was performed on a BD LSRII analyzer and analyzed with CellQuest software.

Primary antibodies for flow cytometry: isotype-control FITC mouse IgG1K, isotype-control PE mouse IgG1K, isotype-control mouse IgG1K, anti-CD105, and -CD166, anti-CD90, anti-CD44; -CD34, anti-CD31, and and -CD106 (BDBiosciences); anti-CD45, anti-CD3, anti-CD4, anti-CD8, anti-CD14, anti-CD15, anti-CD19, anti-CD36, anti-CD56, anti-CD123, and anti-CD235a (eBioscience, Inc.); anti-SMAD3, anti-phosphoSMAD3, anti-SMAD7, anti-JAGGED1 and anti-JAGGED2 (Cell Signaling Technologies, R&D Biosystems, and Santa Cruz Biotechnologies); anti-β-Actin (Sigma-Aldrich, MO, #A-2066).

### Fluorescence Immunocytochemical Analysis (IF)

IF was performed on fixed and permeabilized cells on chamber slides, as described previously in Zwezdaryk (2007), incorporated by reference in its entirety. The primary antibodies were diluted at appropriate concentrations (ratio of 0.5 μg Ab/1×10⁶ cells) and visualized, as standard. Primary antibodies were omitted for negative controls. Micrographs were obtained with a Nikon TE300 inverted epifluorescence microscope. Data were presented as stained micrographs and quantified by ImageJ software densitometry analysis from at least three similarly-stained sections.

### Transforming Growth Factor β (TGF) 1, 2, and 3 Assays

TGFβ secretion was measured from the conditioned medium by luminex immunoassay as per manufacturer's recommendations (LuminexH Bead immunoassay Kit, LINCOplex from Millipore). The MSCs were pre-treated for 1 hr with LPS or poly(I:C), washed, and cultured for an additional 48 hr prior to harvesting the spent medium for TGFβ detection.

Indoleamine 2,3-dioxygenase (IDO) Assay

IDO was measured by real-time PCR analysis of RNA extracted from TLR-primed MSCs incubated for an additional 6 hr prior to RNA harvest. Data are presented by the quantitative comparative CT (threshold value) method, as described previously by Coffelt S B et al. (2009) The pro-inflammatory peptide LL-37 promotes ovarian tumor progression through recruitment of multipotent mesenchymal stromal cells. Proc Natl Acad Sci USA 106: 3806-3811, which is incorporated by reference herein in its entirety.

### HLA-G Expression

HLA-G was detected by both western blot analysis and flow cytometry. Western blot with an anti-HLA-G antibody (clone 4H84), and flow cytometry of both membrane and intracytoplasmic molecules were detected with FITC-conjugated Ab directed against anti-HLA-G1/-G5 isoforms (clone MEM-G/9) or HLAG5 (clone 5A6G7), respectively, as described previously by Rizzo (2008) and incorporated by reference in its entirety.

### Prostaglandin E2 (PGE2) Assay

PGE2 was measured from the spent culture medium—following 1 hr TLR-agonist priming, wash, and 48 hrs of subsequent culture in growth medium—via commercially available ELISA assays (Cayman Chemical, MA).

### Allogeneic Mixed Lymphocyte and MSC Reactions (MLMR)

A variation on methods of Aggarwal (2005) and Krampera (2006), which are incorporated by reference, was used to assess alloreactive T-cell proliferation. Human peripheral blood mononuclear cells (PBMCs) were prepared from leucopheresis packs (The Blood Center, New Orleans, La.) by standard centrifugation on a Ficoll Hypaque density gradient. Ten million PBMCs from at least 5 unrelated donors labeled with carboxyfluorescein diacetate succinimidyl ester (CFSE, Molecular Probes) were resuspended and stimulated with 1 μg of CD3/CD28 antibody beads (Sigma, St. Louis, Mo.) at a 10:1 ratio, in either the presence or absence of isolated TLR-primed MSCs or unprimed MSCs. After 72 hrs, an aliquot was removed for cell counting with trypan blue exclusion as standard, and the remainder of the non-adherent cells were then stained with anti-CD8 or anti-CD4 antibody, and the CFSE dilution of the CD8+ cells assessed by flow cytometry analysis (eBiosciences, Inc.). No fewer than 100,000 events/sample were collected. Cell surface marker expression of CD4/CD8 was assessed and quantified in arbitrary units as mean fluorescence intensity (MFI) of a live population of cells (propidium iodide negative) labeled with a fluorescent-conjugated monoclonal Ab (eBiosciences).

### Statistical Analysis

Typically, data were represented as average±standard error of the mean (S.E.M.). Multiple group comparisons were performed by one-way analysis of variance (ANOVA) followed by the Bonferroni procedure for comparison of means. Comparison between any two groups was analyzed by the two-tailed Student's t-test or two-way ANOVA (Prism4, GraphPad Software Inc. CA). Values of p <0.05 were considered statistically significant.

### Example 1

### Cytokine and Chemokine Secretion Patterns Following TLR3 or TLR4 Activation of hMSCs are Consistent with Divergent Immune Modulating Effects by these Agonists

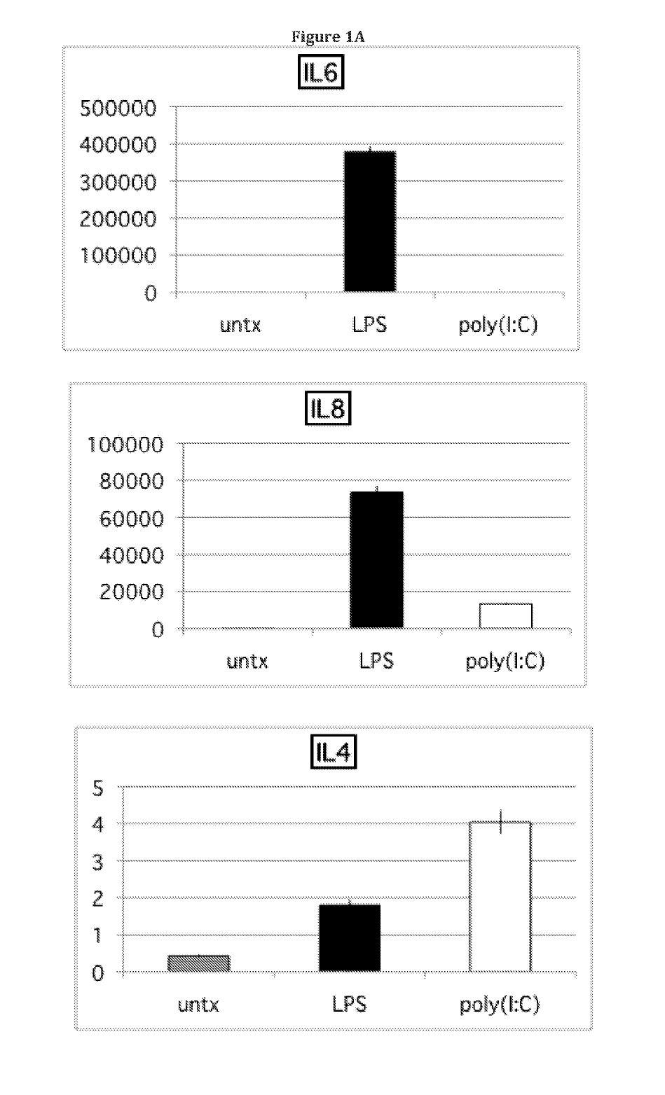

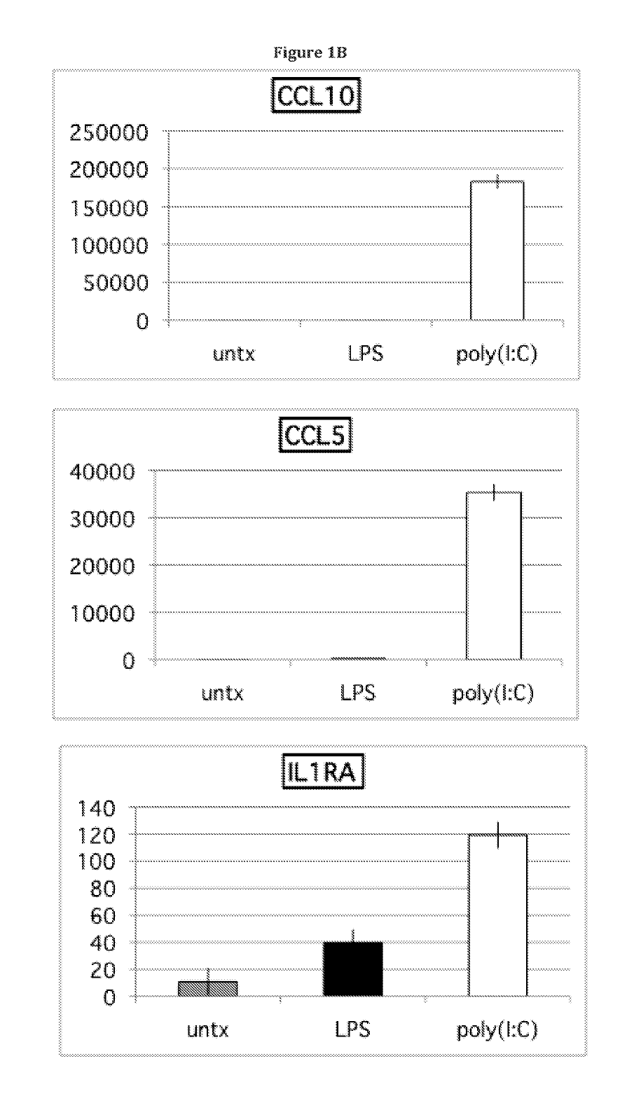

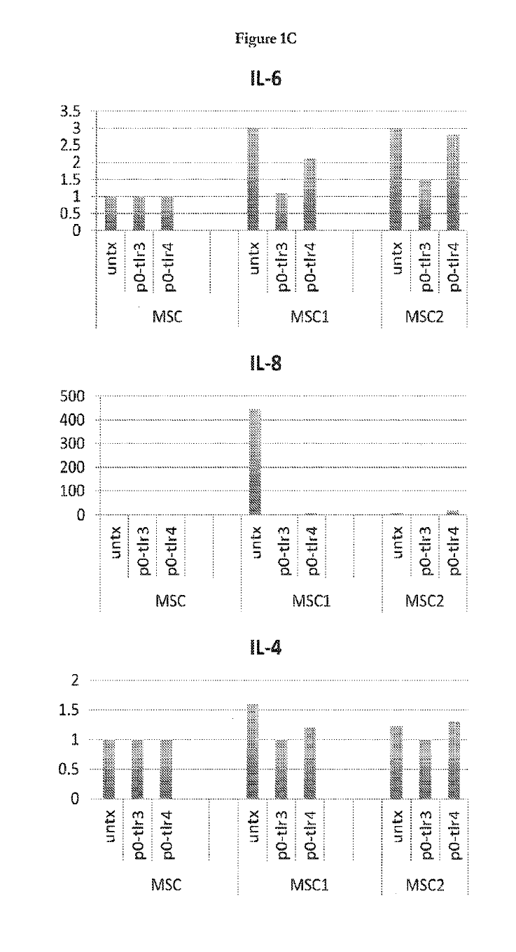

The present disclosure extends previous observations of the effect that TLR signaling has on the immune modulating property of hMSCs, and explains the conflicting reports in this field. The applicant typically used a TLR-priming protocol that comprises incubation with LPS (10 ng/mL) or poly(I:C) (1 μg/mL) added as the hMSCs agonists for TLR4 and TLR3, respectively, for about 1 hr prior to washing, and further 24-48 hr incubation in growth medium. Without wishing to be bound by theory, the incubation time (about 60 minutes) and minimal TLR agonist concentrations used here mimic the gradient of danger signals that endogenous MSCs encounter and respond to at a distance from the site of injury. The conditioned medium was collected and analyzed with Bio-Plex Cytokine Assays (Human Group I & II). TLR3 stimulation in hMSCs led to elevated secretion of certain immune modulating factors different from those elaborated by TLR4 activation in hMSCs (see FIG. 1A). To provide further support for the specific effects by each of these receptors, hMSCs were transfected with dominant negative plasmids for each of the TLR-receptors, and the factors secreted were once again measured by BioPlex assay. This strategy corroborated the TLR3-driven effect on hMSC secretion of CCL10 (IP-10), CCL5 (RANTES), and to a lesser degree IL4 and IL10. It appeared that TLR4 signaling is upstream of IL6 and IL8, as shown in FIG. 1B.

As shown by FIG. 1, MSC1 differ from MSC2 in their secretion of immune modulators. FIG. 1A: The data show increased expression of known immune suppressive factors by TLR3-primed hMSCs (MSC2) but not by TLR4-primed hMSCs (MSC1). For FIG. 1, MSCs were pre-treated for 1 hr with TLR agonists (LPS for MSC1 or poly(I:C) for MSC2), washed and cultured for an additional 48 hr prior to harvesting the spent medium and analysis with Bio-Plex Cytokine Assays (Human Group I & II; Bio-Rad, Hercules, Calif.) following the manufacturer's instructions. Data are presented by the quantitative comparative CT (threshold value) method, as shown by Coffelt S B et al. (2009) Proc Natl Acad Sci USA 106: 3806-3811, which is incorporated by reference in its entirety. Error bars indicate S.E.M. Data are representative of triplicate measurements with 5 MSC donors. FIG. 1B: The data implicate direct TLR3 induction of IP10 (CCL10) and RANTES (CCL5) secretion. Methods: hMSCs were transfected with pZERO-hTLR3 and pZERO-hTLR4 (Invivogen), using 250 ng plasmid/1×10⁶ cells (nucleofector). Cells from each transfection were left untreated or stimulated with TLR3 and TLR4 agonists for 1 hr washed and incubated for 48 hr. Conditioned medium was harvested and analyzed as in FIG. 1A. Transfection efficiency was determined by these methods to be 30-35%. Data are representative of triplicate measurements with 3 MSC donors.

### Example 2

### The Duration of TLR Agonist Exposure Affects Migration and Invasion Capabilities of Treated hMSCs

Apart from the distinct effects of TLR3 and TLR4 activation on cytokine/chemokine secretion, the applicant showed that TLR activation promoted hMSC migration, while Pevsner-Fischer (2007) reported that TLR activation in murine MSCs inhibited the migration of these cells. The hMSC migration assays were performed again, but with varying incubation times. Thus, migration by TLR-primed hMSCs was analyzed following initial exposure to LPS (TLR4 ligand), poly(I:C) (TLR3 ligand), CCL5, or TNFα for an hour or 24 hr prior to loading the cells on the top chamber for transwell migration assays (see, e.g., FIG. 2). Stimulation for 1 hr of TLR3 and TLR4 within hMSCs promoted migration and invasion towards 16.5% serum containing medium when compared to untreated samples. However, 24 hr incubation with these ligands suppressed migration and invasion of the treated hMSCs. By contrast, this longer incubation time was essential for CCL5 and TNFα driven migration and invasion by the hMSCs. Inhibition of the expression of TLR3 and TLR4 receptors by nucleofection with knockdown plasmids diminished migration by >50% in unprimed hMSCs. However, LPS or poly(I:C) treatment of the transfected cells resulted in greater migration when compared with unstimulated controls (data not shown). Without wishing to be bound by theory, the stress of nucleofection and/or the endogenous inhibition of the TLR receptors may derepress a TLR-associated inhibitor of migration, and thus enhance—rather than suppress—the migration of transfected hMSCs as expected. It appears that migration and invasion mechanisms driven by TLRs within hMSCs are more complex than originally appreciated.

The data of FIG. 2 show that short-term TLR-priming stimulates migration. By contrast, 24 hr incubation is needed for enhanced migration by CCL5 (RANTES) and TNFα treatment. To generate the data of FIG. 2, hMSCs migration was examined by transwell migration assay after pre-incubation with TLR-ligands, CCL5 (150 ng/mL), or TNFα (1 ng/mL) for either 1 or 24 hr prior to loading on Matrigel-coated inserts. After overnight incubation, migration towards the serum chemoattractant was visualized and recorded by fluorescence microscopy. Migration was quantified from the obtained micrographs by counting the number of fluorescently-labeled cells remaining after removal of non-migrating cells in triplicate wells. The bar graphs of the obtained results were normalized to unprimed cells. Error bars indicate S.E.M. (n=6).

### Example 3

### Varying Effects of TLR3 and TLR4 Stimulation on hMSCs Adipogenic and Osteogenic Differentiation Potential

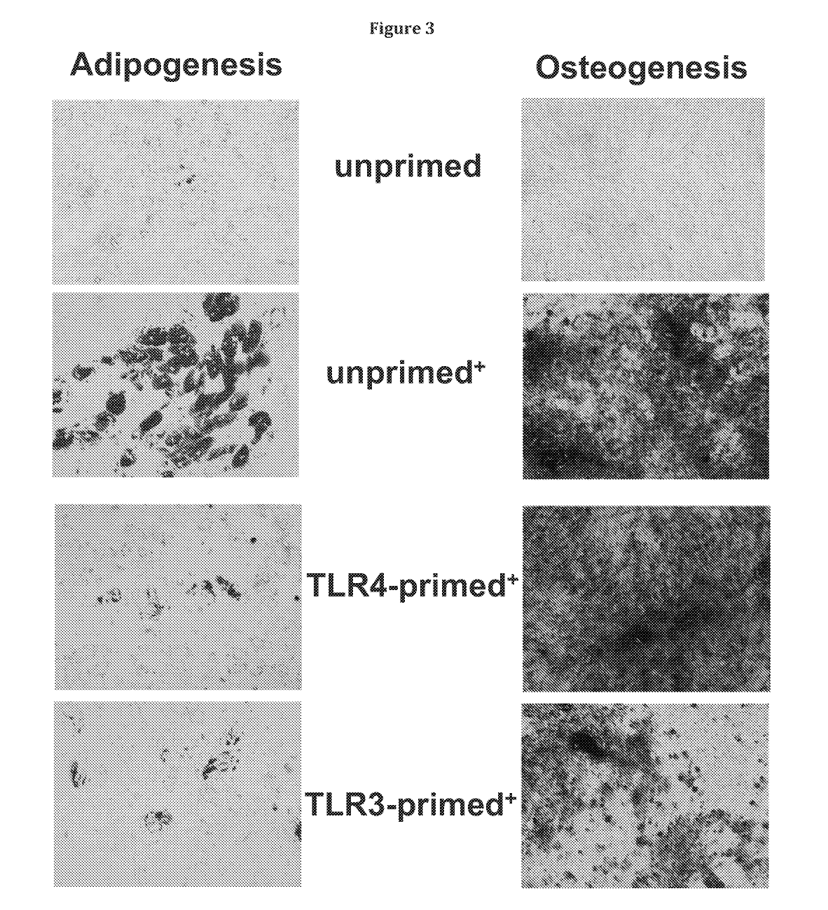

The effect of TLR3 and TLR4 activation on the tri-lineage (cartilage, bone, fat) differentiation capabilities of hMSCs was also measured but, as described above, using reduced amounts of TLR ligand. The hMSCs were simultaneously induced to differentiate in the constant presence of TLR3 (1 μg/mL poly(I:C)) and TLR4 agonists (10 ng/mL LPS) maintained for the duration of the differentiation assays in the inductive medium. With this method, an inhibition of all bone, fat, or cartilage (not shown) programs was noted after TLR3 activation of hMSCs (FIG. 3). Simultaneous TLR4 activation of hMSCs inhibited adipogenesis, stimulated osteogenesis, and did not affect chondrogenesis (not shown).

As shown by FIG. 3, TLR4 activation promotes bone differentiation and inhibits fat differentiation in hMSCs. Methods: The hMSCs were induced (+) to differentiate in the presence of TLR3 and TLR4 ligands throughout the four-week incubation period prior to staining for differentiation markers by established methods. Untreated hMSCs (untx) were either induced (+) or not induced and served as assay controls (n>3).

### Example 4

### TLR3-Stimulated hMSCs Deposit More Fibronectin; TLR4-Stimulated hMSCs Deposit More Collagen

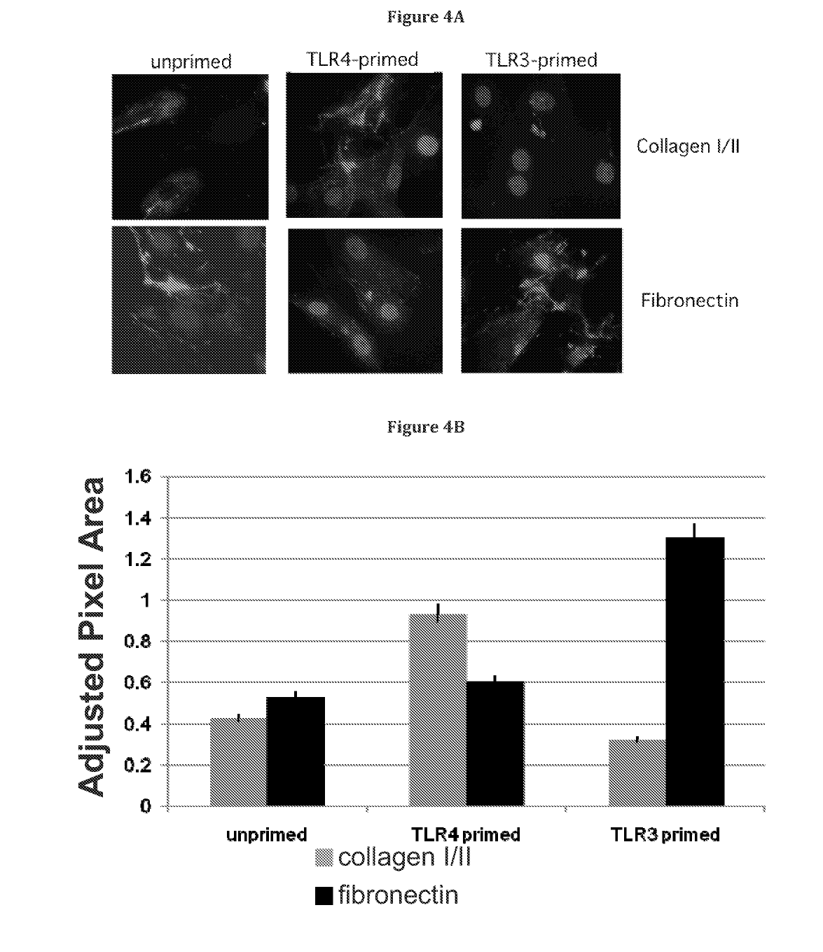

Because of the differing effects on hMSC secretion of cytokines/chemokines, and on hMSC differentiation, all arising from activation of different hMSC TLRs, it was of interest to study whether these different effects extended to another established classical role of hMSCs: extracellular matrix (ECM) deposition. The hMSCs were grown on chamber slides to 70% confluence, primed for 1 hr with TLR3- or TLR4-agonists as before, washed, and then incubated for an additional 24 hr prior to fixation. ECM antibody staining was performed following fixation and membrane permeabilization of the TLR-primed hMSCs seeded on chamber slides (see, e.g., FIG. 4). As a control, the primary antibody was omitted from staining procedure (data not shown). Densitometric analysis revealed that TLR3 stimulation of hMSCs resulted in diminished collagen I/II deposition when compared with unprimed or TLR4-stimulated hMSCs. TLR3 stimulation also resulted in a greater than two-fold increase of fibronectin deposition when compared to unprimed or TLR4-stimulated hMSCs (see FIG. 4B). Interestingly, integrin-linked kinase (ILK) and von Hippel-Lindau protein (VHL), which are also associated with ECM deposition mechanisms, are also differentially expressed after TLR-stimulation. TLR3-stimulation of hMSCs increased the expression of both ILK and VHL, whereas TLR4-stimulation dampened their expression (data not shown).

As shown by FIG. 4, TLR3-primed hMSCs deposit more fibronectin, while TLR4-primed hMSCs deposit more collagen. FIG. 4A: Data demonstrate that TLR4-primed cells deposit twice as much collagen I/II and half as much fibronectin as TLR3-primed cells. FIG. 4B: Densitometric analysis of micrographs in FIG. 4A left bars (grey) are collagen I/II and right bars (black) are fibronectin results normalized to background absorbance. For FIG. 4, hMSCs were grown on chamber slides to 70% confluence pre-treated for 1 h with ligands (1 mM poly(I:C), for TLR3; or 10 ng/mL LPS, for TLR 4) and incubated further for 24 hr prior to fixation. ECM antibody staining was performed following fixation and membrane permeabilization of the TLR-primed or unprimed hMSCs seeded on chamber slides (antibodies from Chemicon International, CA, hu fibronectin MAB1926 and collagen I/II MAB3391). As a control, the primary antibody was omitted from staining procedure (data not shown, n>6). Densitometric analysis of the micrographs was performed with ImageJ software.

### Example 5

### Stimulation of TLR3 Suppresses TGFβ1 and 3 Secretion by hMSCs, but Stimulation of TLR4 does not

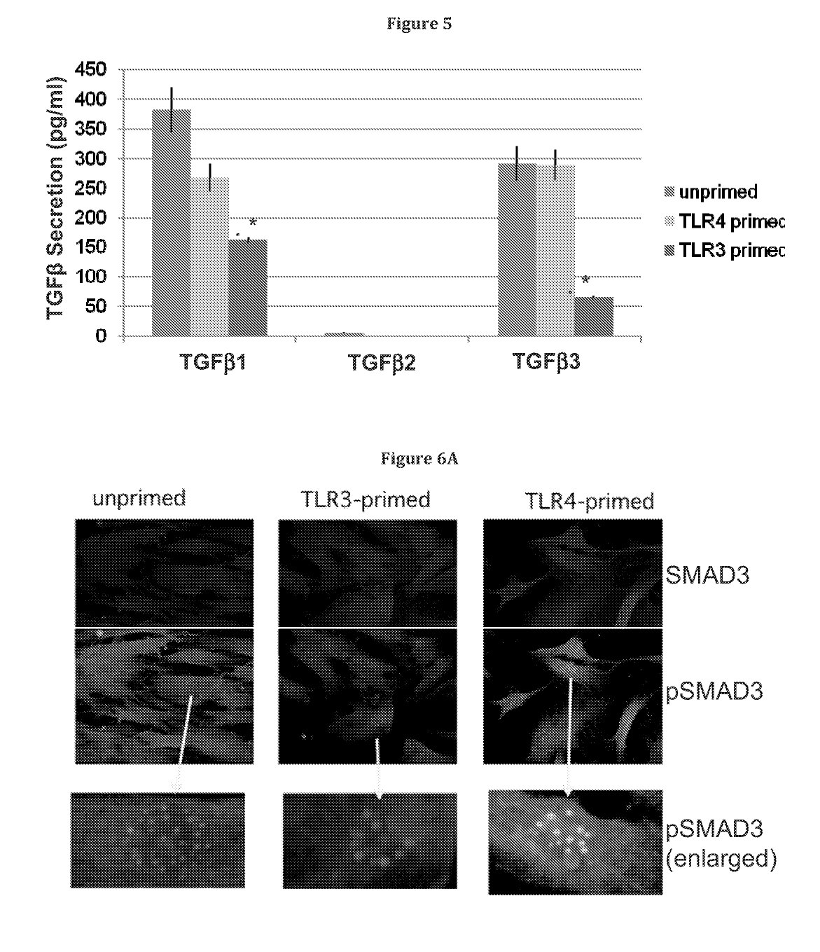

TGFβ secretion by hMSCs was measured from the conditioned medium after TLR3 and TLR4 priming, as before (see, e.g., FIG. 5). TGFβ is known to mediate elevated collagen deposition, as supported by the TLR4-priming results above, and it is also a known immune modulating factor, as shown by Massague J (1990) The transforming growth factor-beta family. Annu Rev Cell Biol 6: 597-641 and by Lee B S et al. (2001) Human leiomyoma smooth muscle cells show increased expression of transforming growth factor-beta 3 (TGF beta 3) and altered responses to the antiproliferative effects of TGF beta. J Clin Endocrinol Metab 86: 913-920, both of which are incorporated by reference. TGFβ1, 2, and 3 were measured from the spent culture medium by luminex immunoassay, per the manufacturer's recommendations (LINCOplex from Millipore). The TLR-primed hMSCs were washed and cultured for an additional 48 hrs prior to harvesting the spent medium for TGFβ detection. TLR3 activation of hMSCs considerably reduced (>65-80%) secretion of TGFβ1 and 3. The levels measured for TGFβ2 secretion were small for all samples (5 pg/mL), and were reduced by both treatments (<1 pg/mL, data not shown). TLR4 stimulation of hMSCs led to little or no change over the untreated samples for this parameter (data not shown).

As shown by FIG. 5, transforming growth factor β (TGFβ1 and 3) expression is diminished in TLR3-primed MSCs compared with measured levels for TLR4-primed and unprimed MSCs. TGFβ2 levels are small, but are further repressed by both treatments. MSCs were pre-treated for 1 hr with TLR4 agonist (LPS for MSC1) or TLR3 agonist (poly(FC) for MSC2), washed, and cultured for an additional 48 hr prior to harvesting the spent medium for TGFβ detection. TGFβ1, 2 and 3 were detected by luminex immunoassay (LuminexH Bead immunoassay Kit, LINCOplex from Millipore). Data are representative of triplicate measurements with six hMSC donors. Error bars indicate S.E.M. *p<0.005 comparison to unprimed MSCs.

### Example 6

### SMAD3 and SMAD7 are Differentially Expressed after TLR3 and TLR4 Priming of hMSCs

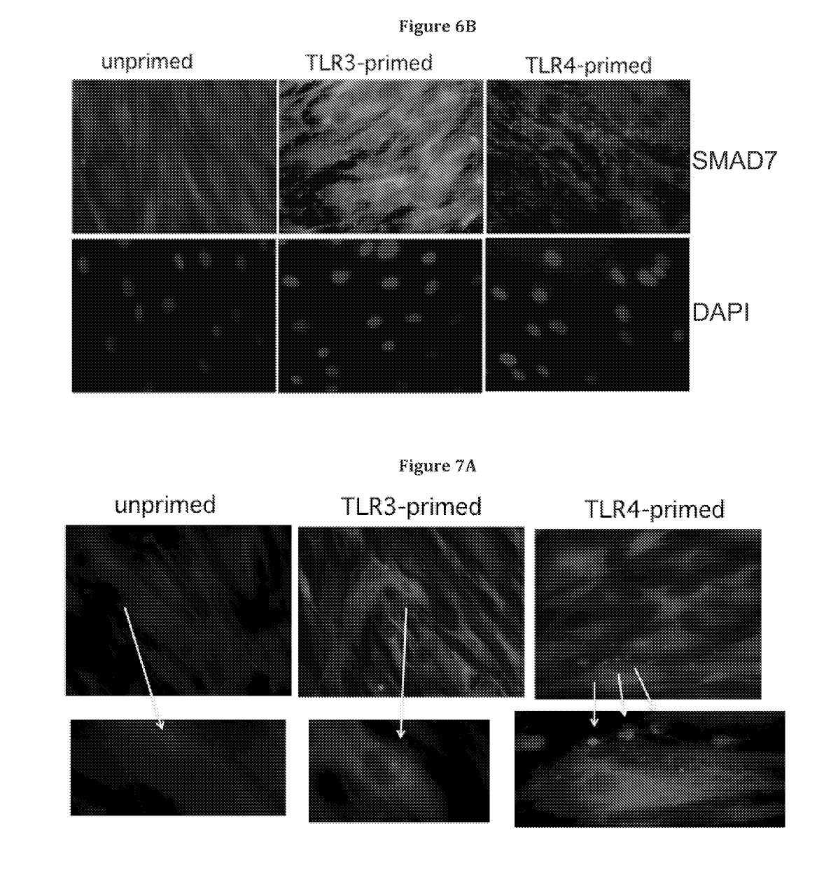

The downstream TGFβ effectors SMAD3 and SMAD7, which may support the TGFβ results presented above, were measured after TLR stimulation of hMSCs. The hMSCs were grown on chamber slides to 70% confluence, pre-treated for 1 hr with TLR3 and TLR4 agonists, washed, and incubated further for 24 hrs prior to fixation. Fluorescently labeled SMAD3, phospho-SMAD3 (activated SMAD3), and SMAD7 antibodies were incubated with the TLR-primed hMSCs as indicated (see FIGS. 6A & B). As a control, the primary antibody was omitted from staining procedure (data not shown). Densitometric analysis revealed that TLR3 stimulation of hMSCs resulted in elevated SMAD7 expression, and diminished and diffused nuclear phospho-SMAD3 and SMAD3, whereas TLR4 stimulation led to increased focused nuclear phosphoSMAD3 expression and reduced SMAD7 expression, when compared with untreated hMSCs. Flow cytometric analyses of these markers supported these observations (data not shown).

SMAD3 expression and activation (phosphoSMAD3, pSMAD3), as well as SMAD7 expression in hMSCs, is shown in FIG. 6. FIG. 6A: Data show that SMAD3 is activated in TLR4-primed (increased nuclear pSMAD3) but not TLR3-primed hMSCs. Arrows point to corresponding magnified cell nuclei. FIG. 6B: SMAD7 expression is induced after TLR3 but not TLR4 stimulation of hMSCs. hMSCs were grown on chamber slides to 70% confluence, TLR-primed as before, and incubated further for 24 hrs prior to fixation. SMAD3, SMAD7, and phosphoSMAD3 antibody staining was performed as indicated above. FIG. 6 shows representative micrographs from five tested hMSC donors.

### Example 7

### TLR3 and TLR4 Stimulation of hMSCs Affects Expression of Jagged 1 and 2

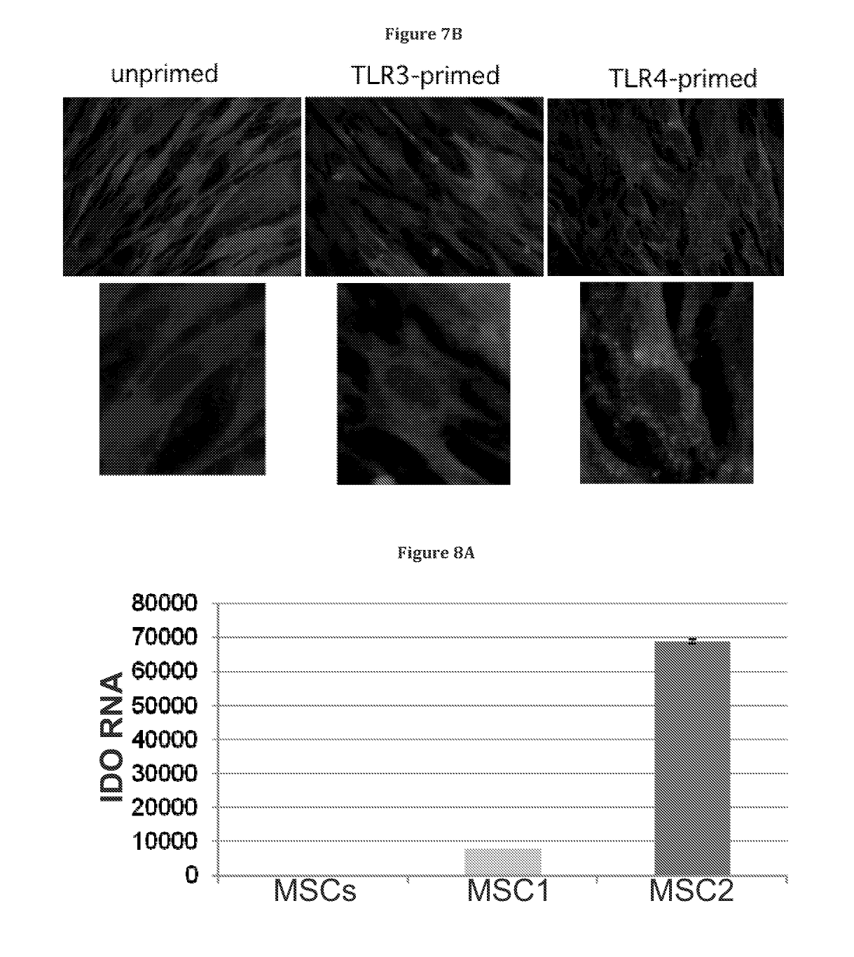

Jagged 1 and 2 expression in TLR stimulated hMSCs was measured because these proteins are reportedly linked to some of the controversial reports on immunomodulation following TLR activation of MSCs, and are also known to correlate with TGFβ signaling, as shown by Liotta (2008), Blokzijl A et al. (2003) Cross-talk between the Notch and TGF-beta signaling pathways mediated by interaction of the Notch intracellular domain with Smad3. J Cell Biol 163: 723-728, and Samon J B et al. (2008) Notch1 and TGFbeta1 cooperatively regulate Foxp3 expression and the maintenance of peripheral regulatory T cells. Blood 112: 1813-1821. The hMSCs were grown on chamber slides to 70% confluence, pre-treated for 1 hr with TLR3 and TLR4 agonists, washed, and incubated further for 24 hr prior to fixation. Fluorescently labeled Jagged 1 and Jagged 2 antibodies were incubated with the TLR-primed hMSCs, as indicated (see FIG. 7). As a control, the primary antibody was omitted from staining procedure (data not shown). Jagged 1 and Jagged 2 expression was diffuse in unprimed hMSCs. TLR3 stimulation of hMSCs resulted in reduced and perinuclear Jagged 1 expression, and unremarkable Jagged 2 expression. TLR4 stimulation led to increased Jagged 1 expression that was both perinuclear and located to foci along cell edges. Jagged 2 expression for TLR4-stimulated cells had a characteristic endosomal distribution. Flow cytometry analyses of these markers supported some of these observations as shown below.

FIG. 7 shows Jagged 1 and Jagged 2 expression in hMSCs. FIG. 7A shows that Jagged 1 expression is elevated, perinuclear, and focused on edges in TLR4-primed but not TLR3-primed hMSCs. Arrows point to corresponding magnified cell nuclei. FIG. 7B shows that Jagged 2 expression is diffuse in TLR3-primed hMSCs, increased, and perinuclear and endosomal after TLR4 stimulation of hMSCs. hMSCs were grown on chamber slides to 70% confluence TLR-primed as before and incubated further for 24 hr prior to fixation. Jagged 1 and Jagged 2 antibody staining was performed as indicated in Methods. Representative micrographs of five tested hMSC donors.

### Example 8

### Indoleamine 2,3-Dioxygenase (IDO) and Prostaglandin E2 (PGE2) are Also Affected by TLR3 and TLR4 Priming

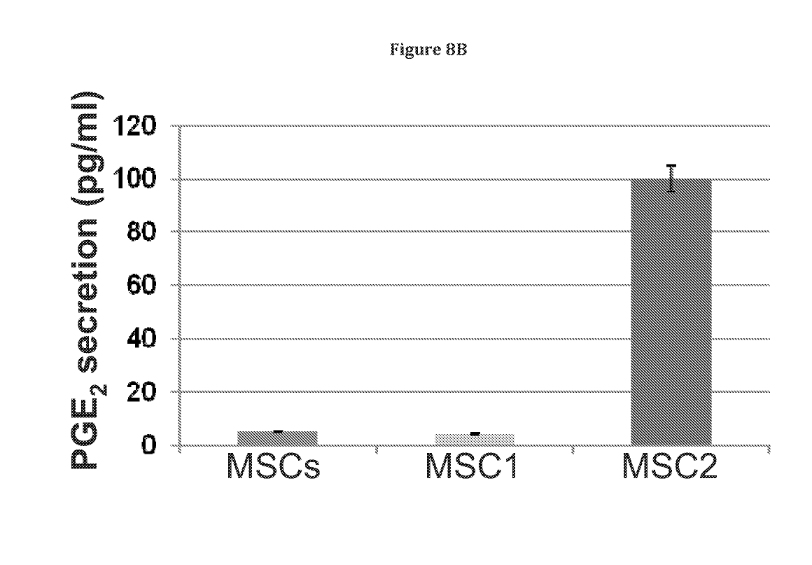

To add support for the observed dichotomy of hMSCs immune modulation downstream from TLR3 and TLR4 stimulation, both indoleamine 2,3-dioxygenase (IDO) and prostaglandin E2 (PGE2) levels were measured following the TLR3- and TLR4 priming protocol, as shown by Fibbe (2007) and Uccelli A et al. (2008) Mesenchymal stem cells in health and disease. Nat Rev Immunol 8: 726-736, which are incorporated by reference in their entireties. Both IDO and PGE2 are known potentiators of hMSC immune modulation. IDO was measured by real-time PCR analysis of RNA extracted from TLR-primed hMSCs incubated further for 6 hrs prior to RNA harvest. Data are presented by the quantitative comparative CT (threshold value) method (FIG. 8A) of Coffelt (2008), which is incorporated by reference in its entirety. PGE2 was measured from the spent culture medium after 1 hr TLR-agonist pretreatment, wash, and 48 hrs of subsequent culture by commercially available ELISA assays (FIG. 8B). Consistent with the previous results, these immunosuppressive indicators are elevated following TLR3 (poly(I:C)) stimulation, and, in contrast, mostly unchanged by TLR4 (LPS) activation of the hMSCs.

FIG. 8. shows that MSC1 differ from MSC2 in their expression of inflammatory mediators. The data show increased expression of known immune suppressive effectors like indoleamine 2,3-dioxygenase (IDO) and prostaglandin E2 (PGE2) by TLR3-primed but not TLR4-primed hMSCs. Methods: MSCs were pretreated for 1 hr with TLR agonists (LPS for MSC1 or poly(I:C) for MSC2), washed, and cultured for an additional 48 hrs prior to harvesting the spent medium for PGE2 detection. PGE2 was measured with commercially available ELISA assays (Cayman Chemical, MA). For IDO measurement, MSCs were primed as described, and incubated another 6 hrs prior to harvesting the RNA for real time PCR assay. Data are presented by the quantitative comparative CT (threshold value) method of Coffelt S B et al. (2009) Leucine leucine-37 uses formyl peptide receptor-like 1 to activate signal transduction pathways, stimulate oncogenic gene expression, and enhance the invasiveness of ovarian cancer cells. Mol Cancer Res 7: 907-915, incorporated by reference in its entirety. Error bars indicate S.E.M. n>3 with at least four different hMSC donors.

### Example 9

### Allogeneic Co-Culture of hMSCs and hPBMCs Leads to T Cell Activation with TLR4 Primed hMSCs, but not Unprimed or TLR3 Primed hMSCs

The immunosuppressive role of heterogeneous MSCs was originally described from allogeneic co-cultures of MSCs with PBMCs or isolated naïve T-cell preparations, as shown by Aggarwal (2005) and Beyth (2005). The addition of unprimed MSC pools to alloreactive T-cells prevents their activation and/or proliferation. Additionally, MSCs infused into allogeneic hosts do not elicit host immune reactivity. This is largely due to the fact that the unprimed MSCs express low levels of human leukocyte antigen (HLA) major histocompatibility complex (MHC) class I, do not express co-stimulatory molecules (B7.1/CD80, B7.2/CD86, CD40, or CD40L), and can be induced to express MHC class II and Fas ligand only upon interferon (IFN) treatment, as shown by Aggarwal (2005) and Krampera (2006).

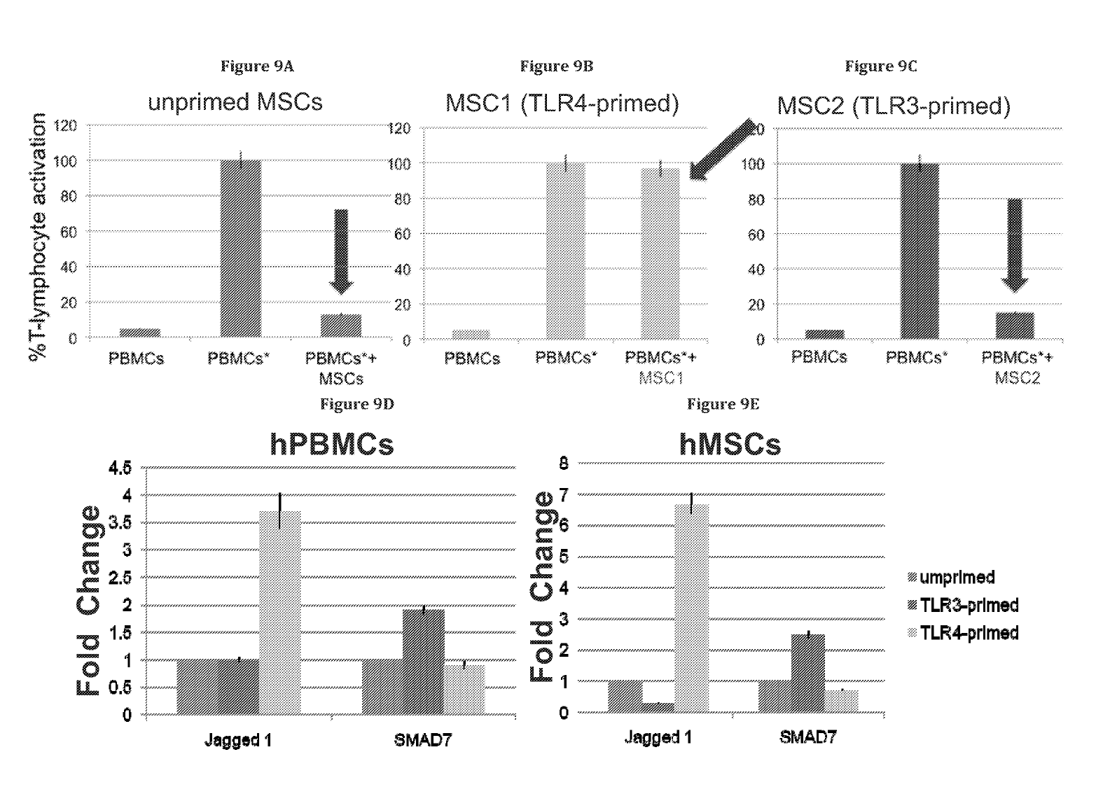

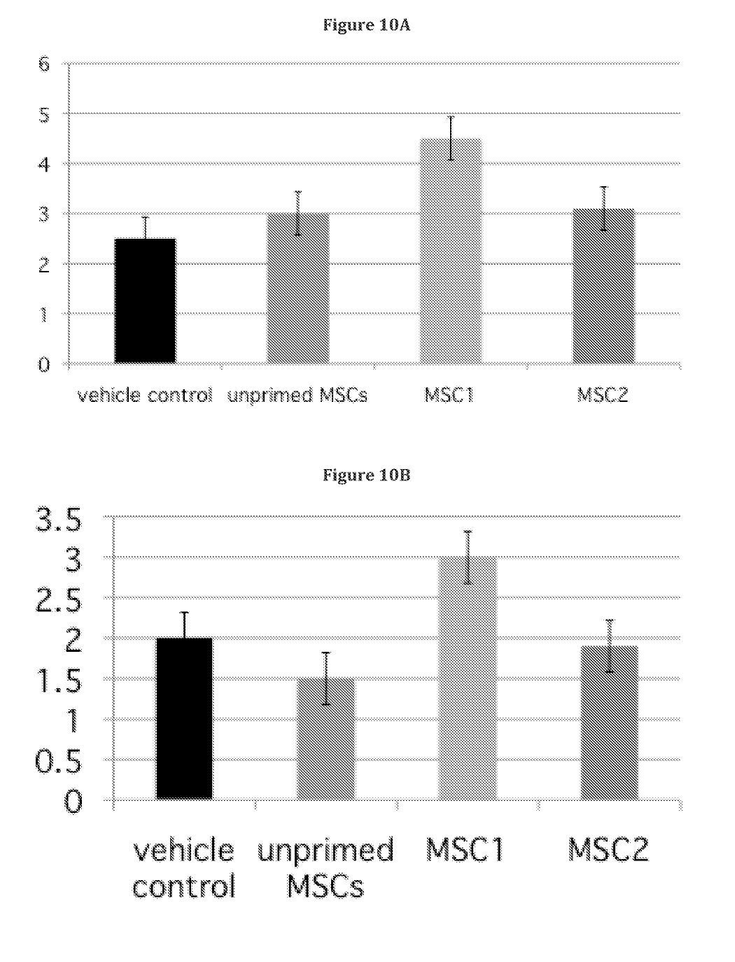

T-lymphocytes among human peripheral blood mononuclear cells (hPBMCs, 10⁶ from at least five unrelated donors, labeled), in the presence or absence of the isolated TLR-primed MSCs or unprimed MSCs, were resuspended and stimulated with 1 μg of CD3/CD28 antibody beads. After 72 hrs, the cells were stained with anti-CD8 or anti-CD4 antibody, and CFSE-label dilution of the CD8+ cells was assessed by flow cytometric analysis. Data are expressed as percent activation or change from the % T-lymphocyte activation obtained for the activated hPBMCs not co-cultured with hMSCs (see FIG. 9).

FIG. 9 shows that MSC1 support PBMC (T cell) activation, while unprimed MSCs and MSC2 suppress it. The data show differences (arrows) in T cell activation when allogeneic PBMCs are stimulated (PBMCs*), and co-cultured with untreated MSCs (FIG. 9A), MSC1 (FIG. 9B) or MSC2 (FIG. 9C). FIGS. 9D & 9E show expression of Jagged 1 and SMAD7 in MLMR co-culture assays. There is elevated Jagged 1 expression in MLMR assays with MSC1 (TLR4-primed), when compared to MSC2 (TLR3-primed), and unprimed assay cultures. By contrast, there is elevated SMAD7 expression in MSC2, when compared to MSC1, and unprimed assay cultures. FIG. 9D shows expression of Jagged 1 and SMAD7 among the CD45+ non-adherent hPBMCs collected at the end of the MLMR experiments. FIG. 9E shows expression of Jagged 1 and SMAD7 among the CD90+ adherent hMSCs collected at the end of the MLMR experiments. T cells among the peripheral blood mononuclear cells (PBMCs) were activated with 1 μg of CD3/CD28 antibody beads, prior to labeling with fluorescent label (CFSE), to monitor their activation or cell division, and loaded at a 10:1 ratio over the hMSCs. The hMSCs were: untreated, primed for 1 hr with TLR-4 (MSC1), or TLR3 (MSC2) agonist; washed in medium; and loaded with the PBMCs. After greater than 72 hrs of co-culture, the CFSE-labeled PBMCs were harvested from the adherent MSCs, stained with propidium iodide to gate for live cells, and measured by flow cytometry. Unstained cells and PBMCs not activated with antibodies served as controls in the assay. Data are expressed as change from the % T cell activation obtained for CD3/CD28 antibody-activated PBMCs not co-cultured with MSCs=100. Error bars indicate S.E.M. Data are averages of triplicate determinations with five MSC donors and two PBMC donors.

As previously reported, incubation of unprimed hMSCs with hPBMCs considerably reduced their activation by >90% (FIG. 9A; see also Aggarwal (2005)). However, TLR4 stimulation inhibited this immune dampening effect by the hMSCs (back to almost 100% activation; see FIG. 9B), while TLR3 supported the immune suppression (>90%), as shown in FIG. 9C. These data demonstrate that TLR-priming effectively polarizes the hMSCs towards two distinct phenotypes. TLR4-priming of hMSCs results in a pro-inflammatory signature referred to here as MSC1; TLR3-priming supports an immune suppressive one referred to here as MSC2. TLR4 activation of hMSCs also consistently resulted in twice as many non-adherent cells recovered at the end of the experiment when compared to the cells recovered from un-activated PBMCs, unprimed hMSC, or TLR3-primed hMSC (see TABLE 1).

As shown by TABLE 1, below, allogeneic co-culture assays reveal that TLR4 priming of hMSCs (MSC1) promotes T-cell proliferation, while unprimed hMSCs and TLR3 primed hMSCs (MSC2) suppress it. Methods: T cells among the peripheral blood mononuclear cells (PBMCs) were activated with 1 μg of CD3/CD28 antibody beads prior to labeling with fluorescence label (CFSE) to monitor their activation or cell division and loaded at a 10:1 ratio over the MSCs for 72 hrs. For cell counts, an aliquot of the 72 hrs spent medium was removed prior to flow cytometry for trypan blue staining and counting as standard. Data are representative of four independent experiments and are expressed as mean cell counts±S.E.M. of four replicate counts on a hemocytometer after trypan blue staining. A total of five MSC donors and five PBMC donors were used in the assay. Two representative donors (d1, d2) are shown.

TABLE 1
Cell counts after hMSC-PBMC co-cultures

| | Primed | Leukocyte | | |
|---|---|---|---|---|
| MSCs | TLR | Activation | PBMCs, d1 | PBMCs, d2 |
| — | — | + | 50,000 ± 1,784 | 30,000 ± 1,774 |
| MSCs, d1 | — | + | 40,000 ± 1,352 | 30,000 ± 1,980 |
| MSC1, d1 | TLR4 | + | 70,000 ± 3,234 | 80,000 ± 5,976 |
| MSC2, d1 | TLR3 | + | 35,000 ± 1,122 | 33,000 ± 1,444 |
| MSCs, d2 | — | + | 50,000 ± 2,354 | 40,000 ± 1,730 |
| MSC1, d2 | TLR4 | + | 70,000 ± 4,376 | 80,000 ± 6,118 |
| MSC2, d2 | TLR3 | + | 30,000 ± 2,974 | 32,000 ± 1,750 |

The expression of various immune modulating factors was measured from the spent co-culture medium at the end of the experiment with BioPlex assays, as described in Coffelt (2009) Proc Natl Acad Sci USA 106: 3806-3811, incorporated by reference herein in its entirety. The expression of CCL5 and CCL10 followed the same patterns as above. Increased secretion for CCL5 and CCL10 was observed in co-cultures with TLR3-primed hMSCs when compared to unprimed or TLR4-primed cultures. By contrast, IL6 and IL8 secretion was higher in the co-culture medium of TLR4-primed cells when compared to the other two groups (data not shown). Jagged 1 and SMAD7 expression within co-cultured cells was measured by flow cytometry (see FIGS. 9D & E). For the purpose of the analysis, CD45+ cells were considered hPBMCs, and CD90+ adherent cells were considered hMSCs. Jagged 1 expression was elevated in both the hPBMCs and hMSCs populations harvested from TLR4-primed MSC co-cultures when compared to unprimed cultures. SMAD7 expression in both was elevated in TLR3-primed MSC co-cultures when compared to unprimed cultures.

### Example 10

### TLR-Regulated Gene cDNA Arrays

The effect of TLR stimulation on gene expression within hMSCs was analyzed using a Human Toll-Like Receptor Signaling Pathway PCR Array (SABiosciences, cat. No. PAHS-018A). Results are shown in TABLE 2, presented as fold changes in gene expression of TLR-primed MSC1 and MSC2 relative to unprimed hMSCs for six different donors.

TABLE 2
Effect of TLR stimulation on gene expression within hMSCs

| Gene | MSC1 | MSC2 |
|---|---|---|
| BTK | 1.461246936 | 3.294364069 |
| CASP8 | 15.77972327 | 1 |
| CCL2 | 258.9985122 | 3191.458118 |
| CD14 | 0.615572207 | 1 |
| CD80 | 1 | 1 |
| CD86 | 1 | 1 |
| CHUK | 2.045424095 | 7.889861636 |
| CLEC4E | 1 | 1 |
| CSF2 | 22.23560879 | 1 |
| CSF3 | 1 | 1 |
| CXCL10 | 29853.24214 | 1009.902289 |
| EIF2AK2 | 6.717851944 | 312.9959111 |
| ELK1 | 18.15632106 | 1 |
| FADD | 18.86447441 | 0.946057647 |
| FOS | 12.04865332 | 1.071773463 |
| HMGB1 | 8.275233382 | 1.117287138 |
| HRAS | 14.71076125 | 1.892115293 |
| HSPA1A | 1338.498679 | 1.802500925 |
| HSPD1 | 19.8903947 | 0.876605721 |
| IFNA1 | 8.543338824 | 1 |
| IFNB1 | 806.7623793 | 37.01402188 |
| IFNG | 1 | 1 |
| IKBKB | 1 | 0.986232704 |
| IL10 | 14.3005626 | 1 |
| IL12A | 6.167988178 | 1.197478705 |
| IL1A | 199.8536346 | 35.26096371 |
| IL1B | 904.1385139 | 99.04415959 |
| IL2 | 1 | 1 |
| IL6 | 829.9036257 | 20.11221399 |
| IL8 | 5066.592205 | 69.5510312 |
| IRAK1 | 20.90244787 | 0.823591017 |
| IRAK2 | 1 | 12.21007367 |
| IRF1 | 539.3956213 | 72.50456866 |
| IRF3 | 1 | 1 |
| JUN | 21.79008622 | 1.931872658 |
| LTA | 15.8718677 | 1 |
| CD180 | 7.166158732 | 1 |
| LY86 | 6.636396111 | 1 |
| LY96 | 45.50647714 | 0.423372656 |
| MAP2K3 | 26.24553316 | 0.768437591 |
| MAP2K4 | 14.04125516 | 0.993092495 |
| MAP3K1 | 27.82674006 | 0.979420298 |
| MAP3K7 | 12.72152747 | 1 |
| MAP3K7IP1 | 12.68630476 | 1.156688184 |
| MAP4K4 | 8.402391453 | 0.979420298 |
| MAPK8 | 25.54908997 | 0.939522749 |
| MAPK8IP3 | 9.540070979 | 1.079228237 |
| MYD88 | 1 | 4.28709385 |
| NFKB1 | 78.85880809 | 3.784230587 |
| NFKB2 | 7.729633444 | 5.897076869 |
| NFKBIA | 73.15071192 | 9.253505471 |
| NFKBIL1 | 28.6011252 | 0.907519155 |
| NFRKB | 13.93267143 | 1.189207115 |
| NR2C2 | 11.90256003 | 1.057018041 |
| PELI1 | 17.83204182 | 1.591072968 |
| PPARA | 10.35890784 | 1.042465761 |
| PRKRA | 11.05325977 | 1.035264924 |
| PTGS2 | 121.1289582 | 2.928171392 |
| REL | 1 | 3.182145935 |
| RELA | 17.20075517 | 2.173469725 |
| RIPK2 | 83.9582389 | 28.4429658 |
| SARM1 | 14.4841187 | 0.460093825 |
| SIGIRR | 1 | 1 |
| SITPEC | 11.94885108 | 1.094293701 |
| TBK1 | 1 | 0.907519155 |
| TICAM2 | 1 | 0.829319546 |
| TIRAP | 16.70722242 | 0.598739352 |
| TLR1 | 1 | 1 |
| TLR10 | 2.984729109 | 2.203810232 |
| TLR2 | 1 | 1 |
| TLR3 | 164.1874448 | 10.77786861 |
| TLR4 | 5.300028068 | 2 |
| TLR5 | 1 | 1 |
| TLR6 | 11.03182828 | 1.515716567 |
| TLR7 | 1 | 1 |
| TLR8 | 1 | 1 |
| TLR9 | 20.84457425 | 1 |
| TNF | 42.83742928 | 9.063071082 |
| TNFRSF1A | 1 | 1 |
| TOLLIP | 30.85003027 | 0.882702996 |
| TRAF6 | 48.7591138 | 7.06162397 |
| TICAM1 | 42.13070055 | 3.182145935 |

### Example 11

### BioPlex Human Cytokine, Chemokine and Growth Factor Assays

hMSCs were pre-treated for 1 hr with TLR agonists (LPS for MSC1; poly(I:C) for MSC2), washed and cultured for an additional 48 hrs prior to harvesting the spent medium and analysis with Bio-Plex Cytokine Assays following the manufacturer's instructions. Data are presented in TABLE 3, expressed in average pg/mL obtained from corrected triplicate measurements with at least 3 MSC donors in four independent experiments. Dominant negative transfected plasmids used were pZero-TLR3 (p0-TLR3) and pZero-TLR4 (p0-TLR4, InvivoGen, San Diego, Calif.).

TABLE 3
BioPlex Human Cytokine, chemokine and growth factor assays

| Growth | | | | unprimed- | unprimed- | MSC1- | MSC2- | MSC2- | MSC2- |
|---|---|---|---|---|---|---|---|---|---|
| Factor | unprimed | MSC1 | MSC2 | p0-TL3 | p0-TL4 | p0-TL3 | p0-TL4 | p0-TL3 | p0-TL4 |
| IL1ra | 11.1 | 39.7 | 120.2 | | | | | | |
| IL2R | 0 | 0 | 41.3 | | | | | | |
| IL4 | 0.5 | 1.71 | 3.99 | 3.99 | 6.67 | 3.55 | 7.79 | 6.65 | 8.54 |
| IL6 | 414 | 7,287 | 39,987 | 14,416 | 9,734 | 15,787 | 9,434 | 22,026 | 13,713 |
| IL8 | 45 | 6,998 | 71,233 | 13,055 | 11,432 | 22,533 | 9,837 | 20,345 | 12,994 |
| IL10 | 32.8 | 39.5 | 33.6 | 1.96 | 2.03 | 0.7 | 2.8 | 0.9 | 2.2 |
| IL12p40 | 0 | 0 | 11.5 | | | | | | |
| HGF | 256 | 236 | 187.9 | | | | | | |
| IFN | 66.3 | 336.9 | 699.4 | | | | | | |
| CCL10 | 0 | 413.3 | 181,777 | 861 | 2,290 | 779 | 2,642 | 1,799 | 24,696 |
| CCL5 | 15.7 | 297.4 | >35,999* | 2,594 | 3,540 | 1,415 | 3,076 | 4,246 | 14,806 |
| TNF-alpha | 8.1 | 51.8 | 501.3 | 431 | 3,39 | 411 | 418 | 460 | 481 |
| TNF-alpha | 5.5 | 4.5 | 3.7 | | | | | | |
| VEGF | 2,058 | 3,213.7 | 2,713 | | | | | | |

*level for MSC2 was above limit of detection in some of the assays

Toll-like receptors (TLRs) are vital for coordinating not only the pro-homeostatic tissue injury responses of immune cells, but also that of multipotent mesenchymal stromal cells (MSCs) of various origins. In trying to tease out the molecular details of TLR signaling within human MSCs (hMSCs), distinct effects after stimulation of TLR3 were observed when compared with TLR4 activation using a short-term, low-level TLR priming protocol. Using that protocol in the examples presented here (see, e.g., EXAMPLE 1), the present disclosure shows that TLR3 stimulation of hMSCs produces immunosuppressive effects, while TLR4 activation of hMSCs provides a pro-inflammatory signature. These observations suggest that these heterogeneous cells can be induced to polarize into two distinct but homogeneously acting phenotypes. Many of the conflicting reports on the net effect of TLR stimulation within stem cells can be resolved by taking into consideration the source of the cells, their originating species, and the duration and concentration of TLR agonist exposure, as the present disclosure shows. Consistent with this and the new MSC paradigm, the present examples demonstrate that short-term, low-level exposure with TLR4 agonists polarizes hMSCs toward a pro-inflammatory MSC1 phenotype important for early injury responses. By contrast, the downstream consequences of TLR3 agonist exposure of hMSCs are its polarization toward an immunosuppressive MSC2 phenotype essential to later anti-inflammatory responses that help resolve the tissue injury.

The instant disclosure demonstrates that TLR3 mediates elevated secretion of CCL10 (IP10), CCL5 (RANTES), and IL10, since this effect could be specifically inhibited by dominant-negative TLR3 expression and not TLR4-dominant negative expression (see FIG. 1B). However, the enhanced IL6 and IL8 expression after TLR-priming was downstream of both TLR3 and TLR4 activation, and the secretion of other soluble mediators was indirectly affected by these because no direct effect was noted by the dominant negative strategy (see FIG. 1B; note: IL4 and data not shown). All of the siRNA-driven TLR3 inhibition strategies attempted were unsuccessful, owing to the fact that double stranded RNAs used as the interfering agent are most likely also acting as the agonist for the targeted TLR3 receptor. Inhibition of the expression of TLR3 and TLR4 receptors by nucleofection with knockdown plasmids reduced NF- [symbol] B-driven luciferase expression by >90% (data not shown), along with the effect on the soluble mediators. As shown by FIG. 2, hMSC migration is affected by both the stimulant and the time it is exposed to it. Whereas TLR-priming promoted hMSC migration, the equivalent short-term exposure with TNFα and CCL5 did not promote migration. Conversely, long-term TLR-priming inhibited hMSC migration but was effective for TNFα and CCL5 mediated migration. Thus, it appears that short-term, low-level TLR-priming mimics the gradient of danger signals that endogenous MSCs encounter and respond to at a distance from the site of injury that draws them to the appropriate target. Once the hMSCs arrive at a site spilling large amounts of these danger signals, migration pathways need to be turned off and the reparative programs turned on. Transfection of hMSCs with the dominant negative expressing TLR3 and TLR4 plasmids diminished migration by >50% in unstimulated hMSCs, as expected. However, poly(I:C) or LPS stimulation of these transfected cells resulted in further enhancement of migration when compared with unstimulated controls (data not shown). Specific TLR3 or TLR4 receptor inhibition by the transfected dominant negative expressing plasmids appears to de-repress chemokine or other chemotactic receptors' inhibition downstream from these receptors, while potentiating alternative poly(I:C) or LPS receptors.

Polarization of hMSCs by TLR-priming also appears to affect their programming towards tri-lineage differentiation, and the various reported contrasting effects might also be explained by differences of source, amount, and time of incubation with the TLR-agonists during the induction periods. The effect on hMSC differentiation of low levels of TLR agonists, maintained for the duration of the induction of hMSC differentiation, was measured, demonstrating that stimulation of TLR3 and TLR4 produces divergent effects on hMSCs. By these methods, TLR3 activation inhibited all of the tri-lineage programs (see FIG. 3). TLR4 stimulation of hMSCs inhibited adipogenesis, stimulated osteogenesis, and did not affect chondrogenesis. Others have reported that activation of TLR2 on murine MSCs inhibited both differentiation and migration of muMSCs (see, e.g., Pevsner-Fischer (2007)). Liotta (2008) found no effect of TLR activation on adipogenic, osteogenic, or chondrogenic differentiation in hMSCs. Further, in contrast to the instant disclosure, Liotta (2008) suggested equivalent roles for TLR3 and TLR4 engagement in hMSC immune modulation. Recently, Lombardo (2009) reported that TLR3 and TLR4 engagement within hADSCs increased osteogenic differentiation but had no effect on adipogenic differentiation or proliferation. Lombardo (2009) also report that TLR2, TLR3, and TLR4 activation does not affect the ability of hADSCs to suppress lymphocyte activation, in contrast to the Liotta (2008) report. The instant disclosure demonstrates, however, that activation of specific TLRs affects many aspects of stem cell fate. Unfortunately, a consensus on the effects of TLR stimulation on tri-lineage differentiation of stem cells is not possible since some of the experimental methods in the reports of others lack sufficient detail.

Apart from the effects on differentiation, the TLR-priming protocol of the present methods affected the ability of hMSCs to deposit ECM, another established classical function of these cells. Unlike unprimed hMSCs and TLR4-primed hMSCs that deposited more collagen, TLR3-primed hMSCs deposited more fibronectin (see FIG. 4). To help explain these results, TGFβ was evaluated as an established component of mechanisms that control ECM deposition. TGFβ is also linked to immune modulation, as shown by Massague (1990), Lee (2001), and Wang Y et al. (2008) TGF-beta1/Smad7 signaling stimulates renal tubulointerstitial fibrosis induced by AAI. J Recept Signal Transduct Res 28: 413-428, which are incorporated by reference in their entireties. Indeed, TGFβ, SMAD3, and SMAD7 were affected by TLR-priming of hMSCs (see FIGS. 5 & 6). As expected, enhanced collagen deposition in TLR4-primed hMSCs correlated with TGFβ expression and activation of its downstream signals (phosphoSMAD3). By contrast, TLR3-primed hMSCs that deposited greater levels of fibronectin had decreased TGFβ1 and 3 expression and increased SMAD7 (TGFβ signaling inhibitor) expression. Although one might expect that TGFβ, an immunoregulating factor, would be associated with the TLR3-primed phenotype rather than the pro-inflammatory TLR4-primed one, it is likely that TGFβ plays a smaller role in hMSC immunomodulation than for immune cells. Immune modulatory mechanisms of hMSCs may rely more on local IL10 receptor mechanisms, as illustrated recently by Nemeth (2010), Nemeth K et al. (2009) Bone marrow stromal cells attenuate sepsis via prostaglandin E(2)-dependent reprogramming of host macrophages to increase their interleukin-10 production. Nat Med 15: 42-49, and Gur-Wahnon D et al. (2009) The induction of APC with a distinct tolerogenic phenotype via contact-dependent STAT3 activation. PLoS One 4: e6846, which are incorporated by reference in their entireties. Immunomodulation mechanisms controlled by TGFβ appear very complicated and, like TLR-signaling, their effects are dependent upon specific cellular contexts. For instance, in a recent study, investigators sought to quell inflammation in the brain by manipulation of TGFβ and SMAD3 in immune cells as a new method to prevent Alzheimer's disease. Their strategy—surprisingly—increased macrophage infiltration in the brain periphery in direct contrast to their original hypothesis, but fortuitously these cells more effectively cleared amyloid plaques, as shown in Town T et al. (2008) Blocking TGF-beta-Smad2/3 innate immune signaling mitigates Alzheimer-like pathology. Nat Med 14: 681-687.

The TGFβ immune dampening effects are also associated with the reprogramming of T-lymphocyte effector cells towards immunosuppressive T-regulatory cells (T-regs). TGFβ cooperates with members of the Notch1 family to regulate the critical transcription factor (Foxp3) to favor T-regs. Additionally, hMSCs are known to recruit and support T-regs as part of their immune-dampening effects, as shown by Selmani Z et al. (2008) Human leukocyte antigen-G5 secretion by human mesenchymal stem cells is required to suppress T lymphocyte and natural killer function and to induce CD4+ CD25high-FOXP3+ regulatory T cells. Stem Cells 26: 212-222 and Di Ianni M et al. (2008) Mesenchymal cells recruit and regulate T regulatory cells. Exp Hematol 36: 309-318, both of which are incorporated by reference in their entireties. TLR3 and TLR4 signaling within MSCs was recently shown to down-regulate the Notch1 receptor family member, Jagged 1, and by this method to inhibit T-cell suppression by MSCs (see Liotta (2008)). By contrast, with the instant TLR-priming protocol, Jagged1 expression was elevated in TLR4-primed hMSCs, and dampened only in unprimed or TLR3-primed hMSCs. Varied concentrations and incubation durations with the TLR-agonists might help explain these differences. Apart from the distinct TLR-driven migration and soluble immune modulators' effects of hMSCs, the present disclosure shows differences in the expression of IDO and PGE2 secretion (FIG. 8). TLR3-primed hMSCs elaborated elevated levels of both of these when compared with unprimed or TLR4-primed hMSCs. These observations lend further support for the proposed polarization scheme.

The immune modulating effect by the TLR-primed cells on T-lymphocyte activation was investigated (FIG. 9). In light of the conflicting reports noted above on the effect of TLR3 and TLR4 stimulation on MSCs' ability to suppress T-lymphocyte activation, it was of interest to see what effects our TLR-priming protocol had on this hMSC function. Critical to the main premise of this study, it was found that TLR4-primed hMSCs behaved as Liotta et al. reported, and inhibited the recognized MSC suppression of T-lymphocyte activation. While in the applicant's hands, TLR3-primed hMSCs and unprimed MSCs suppressed T-lymphocyte activation, as expected. Consistent with this proposed new polarization MSC paradigm, TLR4-primed hMSCs (MSC1) would support a proinflammatory environment including the T-effector cells found in that environment whereas TLR3-primed MSC2 would favor an immunosuppressive one. In support of this assertion, murine models with inflammatory lung injury were treated with MSC1 and MSC2 cells, and found by several parameters that, as expected, MSC1 treatment aggravated the inflammatory injury, whereas MSC2 improved it, when compared with unprimed hMSC treatments. For the T-lymphocyte activation set of experiments, the classical allogeneic co-cultures were performed with direct contact between hMSC-hPBMCs. The potential of soluble mediators alone in this context was not addressed. For human-derived MSCs, cell-cell contact appears to be essential to their immunomodulatory mechanisms, as shown by Beyth (2005) and Gur-Wahnon (2009), incorporated by reference in their entireties. Indeed, contact-dependent secretion by hMSCs of CCL10 (IP-10), CCL5 (RANTES), HGF, and GM-CSF in third party co-cultures with ovarian cancer cell lines and hMSCs was found (data not shown). In the direct cell contact co-cultures performed here, it was noted that the secretions of these factors followed the same trends, and are consistent with those reported for the hMSCs cultured alone. This finding does not readily explain the contrasting effects by the TLR-primed hMSCs on T-lymphocyte proliferation since IL2 levels or other potential T-cell activating factors were not measured. More information regarding these effects may be gained from animal disease models where both MSCs and leukocytes (PBMCs) interact and can be more directly tested. Alternatively, a better handle on the molecular details for the important contributions of each TLR-primed cell may be provided in studies using individually marked cell compartments specifically knocked-out for distinct genes.

Following overnight incubation of the co-culture assays (and throughout the subsequent experiment), TLR4-primed hMSCs were observed to be more readily coated with the round hPBMCs, as compared with unprimed or TLR3-primed hMSCs. The cell count for this sample group was always greater than that for the other two sample groups (see TABLE 1). This observation is consistent with an increase in this sample group of T-cell activation, as reported in FIG. 9.

Only an immunosuppressive phenotype has been recognized for current heterogeneous MSC preparations until now, potentially because of the manner in which MSCs are isolated from the host and the way they are expanded in ex vivo culture. One reason may be that the default MSC phenotype is an immunosuppressive one, in order to avoid profound and deleterious consequences from a pro-inflammatory MSC1 phenotype in the context of the hematopoietic stem cells (HSCs) that MSCs maintain and support within the progenitor/stem cell niches that both of these cell types share. Circulating or quiescent stem/progenitor cells may be equipped to respond to environmental cues, but may not actively engage immune cells or repair cells while circulating throughout the body or while maintaining HSCs in the bone marrow niche. In a manner analogous to the immature state maintained for monocytes, dendritic cells, and other immune cells that await a triggering signal, MSCs are immunosuppressive until a pro-inflammatory role is essential to promote tissue repair. Additionally, TLR4-priming may not be the optimal way to induce the MSC1 phenotype. It is likely that a combination of other factors (e.g., interferons, or contact with other pro-inflammatory cells and their microenvironments) will more readily induce the MSC1 phenotype, as suggested by Romieu-Mourez R et al. (2009) Cytokine modulation of TLR expression and activation in mesenchymal stromal cells leads to a proinflammatory phenotype. J Immunol 182: 7963-7973, incorporated by reference in its entirety.

In summary, hMSCs polarize into two distinctly acting phenotypes following specific TLR-activation. TLR3-priming specifically leads to enhanced fibronectin deposition, expression of immune dampening mediators, and maintained suppression of T-cell activation. By contrast, TLR4-priming results in collagen deposition, expression of pro-inflammatory mediators, and a reversal of the MSC-established suppressive mechanisms of T-cell activation. The present disclosure challenges current dogma that infused MSCs are only immunosuppressive, demonstrating instead that they are capable of far more complex immune modulating activity. These findings also provide an explanation for some of the conflicting reports on TLR-activation and its consequence on immune modulation by stem cells.

### Example 12

### MSC1 Aggravate the Inflammatory Insult in a Mouse Lung Injury Model

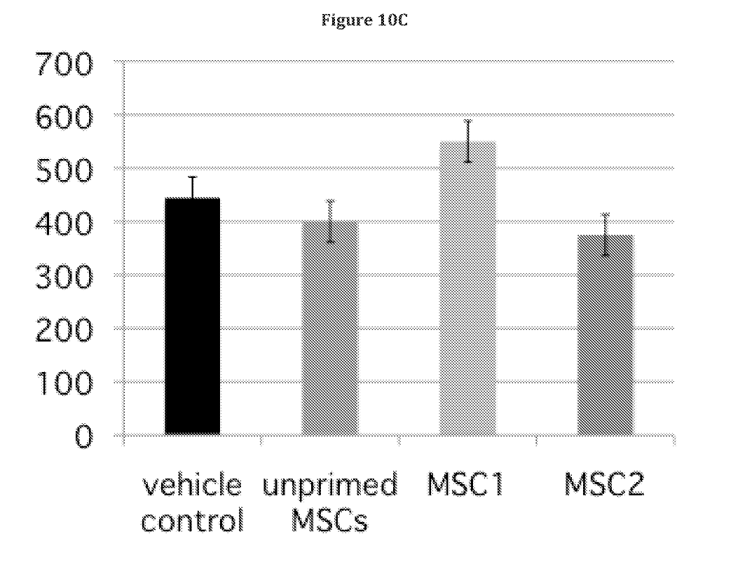

An established endotoxin-induced acute lung injury mouse model was used, whereby LPS or endotoxin (0.1 mg/kg) was instilled intratracheally into adult Balb/C mice. Twenty-four hours later, mice were treated intratracheally with 500,000 unprimed MSC, MSC1, or MSC2, or with HBSS vehicle (at least 3 mice were used per group). For these data, MSC1 are defined as unprimed MSCs incubated for 1 hr with 10 ng/mL LPS, and MSC2 are defined as unprimed MSCs incubated for 1 hr with 1 μg/mL poly(I:C). These are then washed in growth medium and used in the experiments as described. Twenty-four hours after treatment, the animals' lungs were lavaged and the bronchioalveolar lavage fluid (BALF) collected. To characterize the inflammatory response, the collected BALF was analyzed for changes in neutrophil/monocyte recruitment (myeloperoxidase activity), total cell content by flow cytometry, and lung integrity by total protein leaked into the BALF (see FIGS. 10A, 10B, 10C). Error bars indicate SEM. Data are expressed as averages of triplicate determinations from each sample. These data demonstrate that MSC1-therapy aggravated the disease and resulted in increased neutrophil recruitment and more compromised lungs than the conventional MSC or MSC2 therapy.

### Example 13

### MSC1 Support PBMC (T Cell) Activation, while Untreated MSCs and MSC2 Suppress Activation

T cells among the peripheral blood mononuclear cells (PBMCs) were activated with 1 μg of CD3/CD25 antibody beads prior to labeling with fluorescence label (CFSE) to monitor their activation or cell division and loaded at a 10:1 ratio over the MSCs. The MSCs were untreated or primed for 1 hour with TLR-4 (MSC1) or TLR-3 (MSC2) agonist, washed in medium and loaded with the PBMCs. After at least 72 hours of co-culture, the CFSE-labeled PBMCs were harvested from the adherent MSCs and stained with propidium iodide to select for live cells and then measured by flow cytometry. Unstained cells and PBMCs not activated with antibodies served as controls in the assay. Data are expressed as change from the % T cell activation obtained for CD3/CD25 antibody; activated PBMCs not co-cultured with MSCs=100. Error bars indicate SEM. Data are averages of triplicate determinations with 5 MSC donors and 2 PBMC donors.

### Example 14

### MSC1 differ from MSC2 In Expression of Inflammatory Mediators

As shown in FIG. 8, MSC2, but not MSC1, show increased expression of known immune-suppressive effectors, including indoleamine 2,3-dioxygenase (IDO) and prostaglandin E2 (PGE₂). MSCs were pre-treated for 1 hour with TLR agonists (10 ng/mL LPS for MSC1, and 1 μg/mL poly (I:C) for MSC2), washed, and then cultured for an additional 48 hours, after which the overlying medium was collected for detection of PGE₂ via ELISA assay (Cayman Chemical, MA). For IDO measurement, MSCs were pre-treated as before, washed, and then cultured for an additional 6 hours before cells were harvested for RNA and subsequent RT-PCR assay. Data are presented by the quantitative comparative CT (threshold value) method (Coffelt S B et al. (2009)). Error bars indicate S.E.M. n>3, with at least four different hMSC donors.

### Example 15

### MSC1 do not Support Tumor Growth Whereas MSC2 Favor Tumor Growth

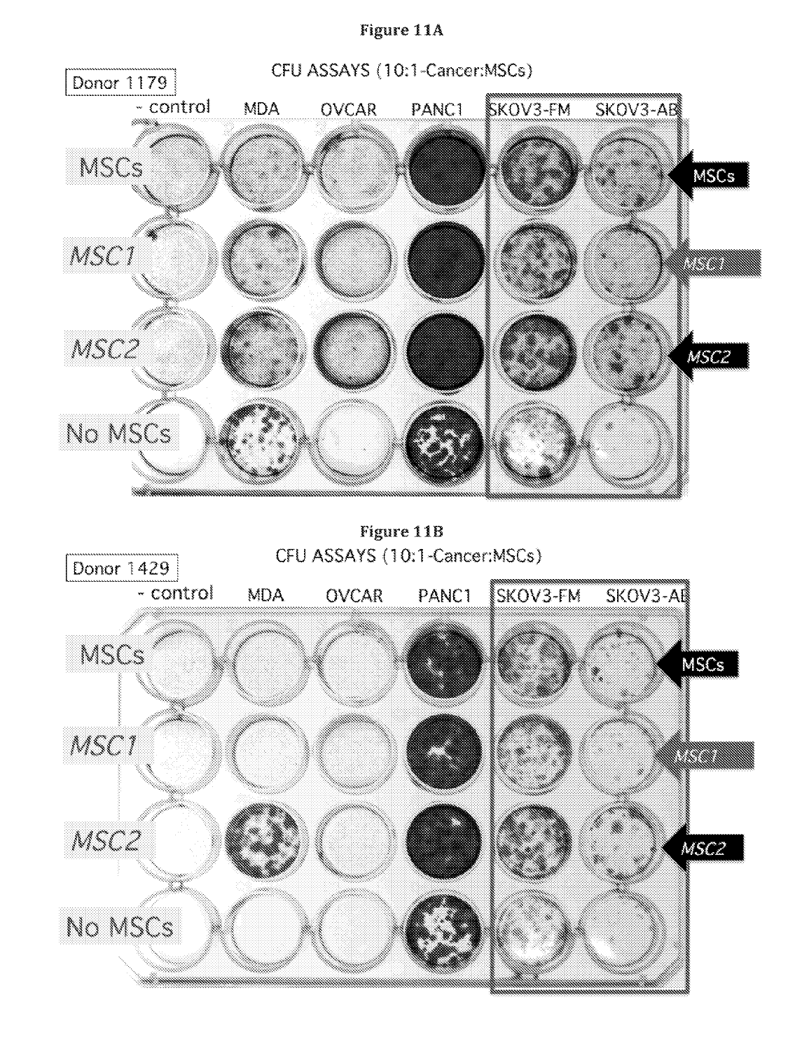

Unprimed MSCs, MSC1, and MSC2 demonstrate distinct effects on colony forming units (CFUs) after coculture with different human cancer cell lines. A CFU assay was performed by culturing human tumor cells (200 cells/well) mixed with unprimed MSCs, MSC1, or MSC2 (2 cells/well) at a ratio of 10 cancer cells per 1 MSC and plated in 24-well plates in growth medium supplemented with 10% FBS as shown in FIG. 11. Cultures were grown for 14 days at 37° C. in a humidified atmosphere of 5% carbon dioxide balance air. Growth medium was changed every 3-4 days. Colonies were visualized by staining with a crystal violet solution (0.5% crystal violet/10% ethanol). The resulting colonies were counted by the colony counting macro in Image) software. Two different human MSC donors were used. Cancer cell lines used were: MDA231-metastatic breast adenocarcinoma; OVCAR-ovarian cancer; PANC-1 pancreatic cancer; SKOV3FM ovarian cancer; and AB ovarian cancer. Micrographs of the stained plates are shown in FIG. 11; colony counts are presented in TABLES 4 and 5 below.

TABLE 4
hMSC donor 1179

| hMSC | Cancer Cell Line | | | | | |
|---|---|---|---|---|---|---|
| type | none | MDA | OVCAR | PANC1 | SKOV/FM | SKOV/AB |

| | | | | | |
|---|---|---|---|---|---|
| untx | 38 | 4 | >100 | 90 | 73 |
| MSC | | | | | |
| MSC1 | 55 | 1 | >100 | 43 | 25 |
| MSC2 | 71 | 10 | >100 | 92 | 65 |
| no MSC | 93 | 2 | 87 | 98 | 44 |

hMSC φ-type: hMSC phenotype;
untx MSC: untreated MSC

TABLE 5
hMSC donor 1429

| hMSC | Cancer Cell Line | | | | | |
|---|---|---|---|---|---|---|
| type | none | MDA | OVCAR | PANC1 | SKOV/FM | SKOV/AB |

| | | | | | |
|---|---|---|---|---|---|
| untx | | 2 | >100 | 85 | 55 |
| MSC | | | | | |
| MSC1 | | >100 | 104 | 27 | |
| MSC2 | 61 | >100 | 119 | 57 | |
| no MSC | | 1 | 102 | 70 | 29 |

hMSC φ-type: hMSC phenotype;
untx MSC: untreated MSC

### Example 16

### Members of the Pro-Inflammatory microRNA155 Family are Elevated in MSC1 and Repressed in MSC2

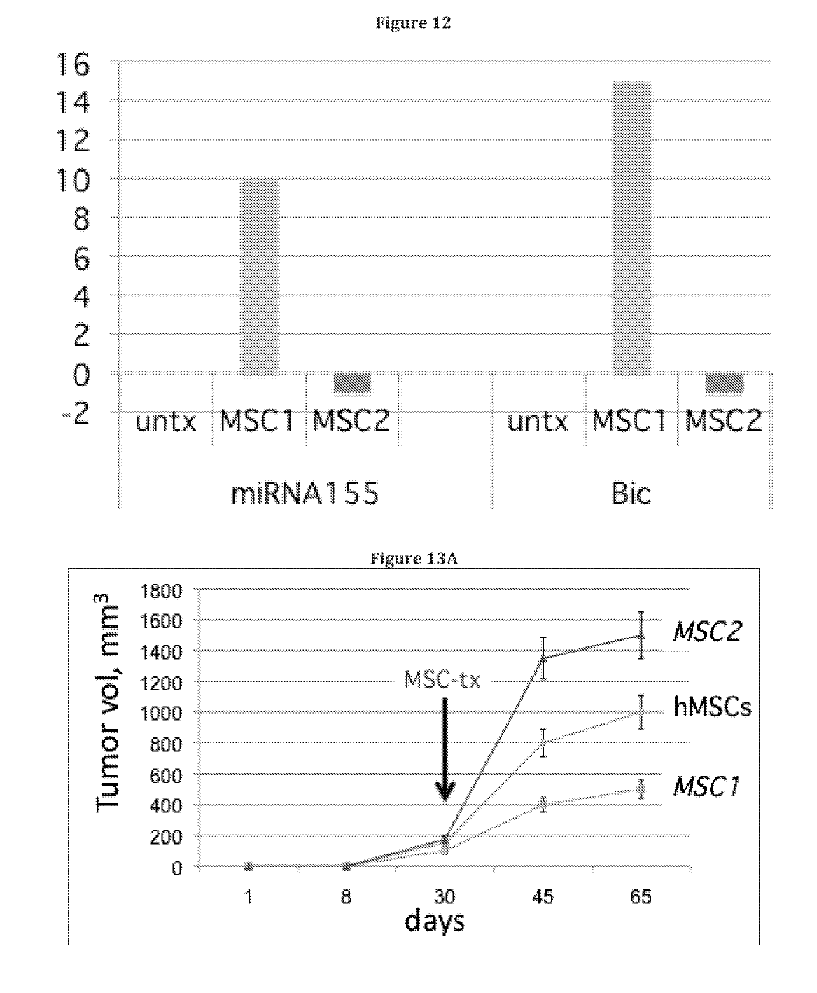

As shown in FIG. 12, levels of microRNA-155 (miRNA-155, or MIR155, which is implicated in inflammatory responses) and its pro-miRNA Bic are elevated in pro-inflammatory MSC1, but suppressed in anti-inflammatory MSC2, versus untreated hMSCs (“untx”). Four different human MSC donors were pre-treated for 1 hour with TLR agonists (10 ng/mL LPS for MSC1, and 1 μg/mL poly (I:C) for MSC2) or left untreated, washed, and then cultured for an additional 48 hours. RNA, along with microRNAs, were extracted by miRNAeasy kit (Qiagen). Quantitative real time primers for miRNA155 and Bic were used in the assay. Data are expressed as percent change from unprimed samples in average cumulative threshold (CT). Samples were run in triplicate for the four donors.

### Example 17

### MSC1 do not Support Tumor Growth Whereas MSC2 Favor Tumor Growth and Metastasis in Mouse Model for Epithelial Ovarian Cancer

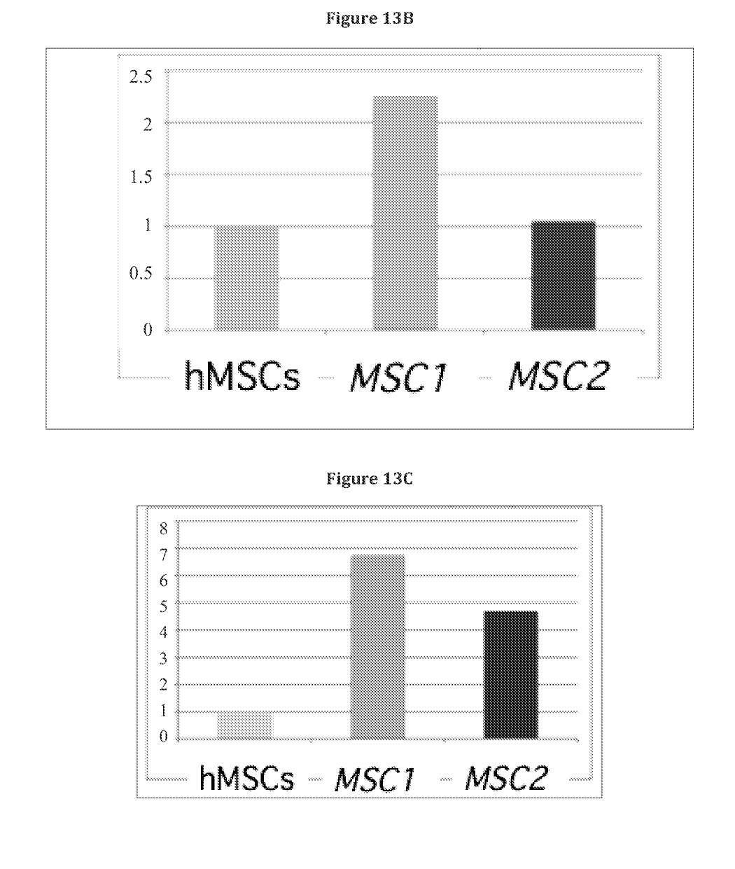

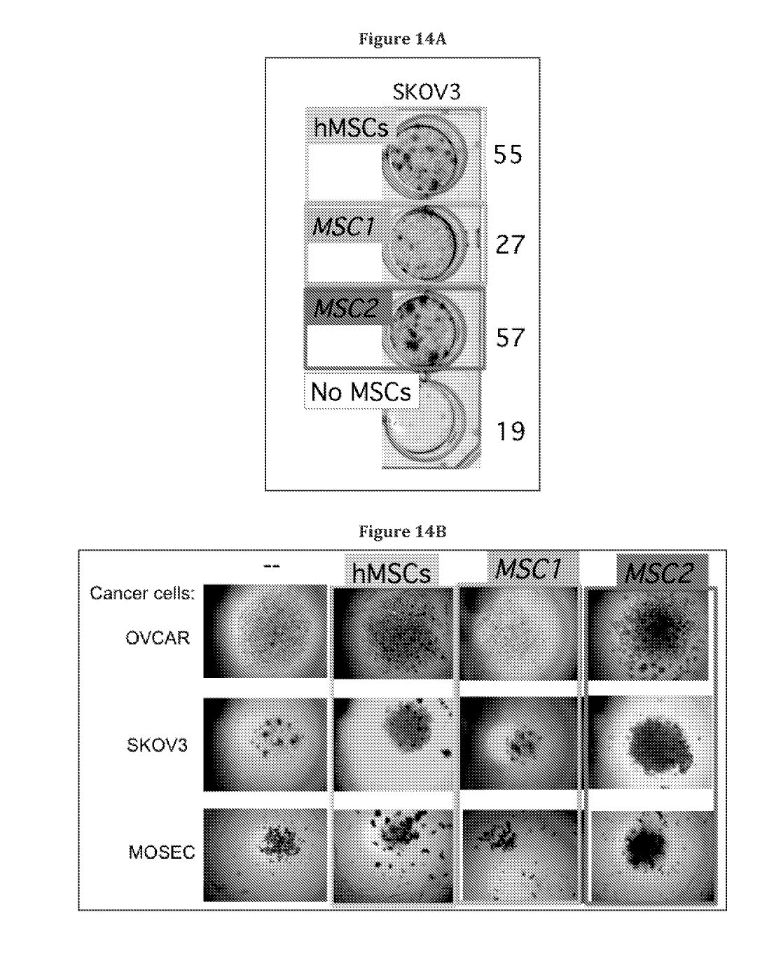

The established syngeneic mouse model for epithelial ovarian cancer used is based upon a spontaneously transformed mouse ovarian surface epithelial cell (MOSEC) line ID8 that has been described in Roby, K. F. et al., Carcinogenesis, 21:585-591 (2000), the disclosure of which is incorporated by reference herein. 4-6 week old female mice (n>10 mice/MSC-treatment) were injected subcutaneously in the right hind leg with 1×10⁷ MOSEC cells. At approximately 4 weeks a single dose of labeled human MSC (hMSCs), MSC1 or MSC2 (1×10⁷/per mouse) were injected i.p. as indicated by the arrow ↓ shown in FIG. 13A. As shown in FIG. 13, there are differences in tumor volume, CD45+ leukocyte and f4/80+ macrophage recruitment after the treatment of mice with established ovarian tumors, with human MSC1 and MSC2 based therapies.

For FIG. 13A, tumor growth was measured with calipers as standard at weekly intervals until day of mouse macrifice (day 65). Harvested tumors and metastasis were weighed, counted and processed for flow cytometry and immunohistochemical analysis (IHC, Coffelt et al., 2009). Metastasis was found only in MSC2-treated mice. MSCs were detect by flow cytometry and IHC. All MSC-treated samples had similar detectable MSCs within the tumor tissue-trending towards more MSC1 and MSC2 measured then hMSCs.

For FIGS. 13B and 13C, approximately 15-25 cells counted per 200× field after 24 hr of MSC-treatment and 2-5 cells at time of tissue harvest (days 65). Sectioned tumor sample slides were stained with murine CD45 for FIG. 13B or F4/80 for FIG. 13C. Antibodies and the number of positively stained immune cells per 200× field were scored as described in Coffelt, S. B. et al., Proc. Natl. Acad. Sci. USA, 106: 3806-3811 (2009), the disclosure of which is incorporated herein by reference. Data are expressed as average cells counted in 4 fields/slide relative to hMSC sample. Data indicate in vivo stability and distinct effects by MSC1 and MSC2. A single delivery of MSC1 based therapy resulted in slower growing tumors, whereas comparable therapy with MSCs or MSC2 resulted in larger tumors and tasitasis at the end of the study (day 65).

### Example 18

### In Vitro Studies Show Divergent Effect of MSC1 and MSC2 on Co-Cultures of Various Human Cancer Cell Lines with MSC1 and MSC2

FIG. 14 shows that MSC1 do not support tumor growth whereas MSC2 favor tumor growth. Data in FIG. 14A demonstrate that there are distinct effects on colony forming units (CFU) after coculture of different human cancer cell lines with untreated MSCs (hMSCs), MSC1, or MSC2. CFU assay was performed by culturing human tumor cells (200 cells/well) mixed with hMSCs, MSC1, or MSC2 (2 cells/well) at a ratio of 10 cancer cells per 1 MSC and plated in 24-well plates in growth medium supplemented with 10% FBS. Cultures were grown for 14 days at 37° C. in a humidified atmosphere of 5% carbon dioxide balance air. Growth medium was changed every 3-4 days. Colonies were visualized by staining with a crystal violet solution (0.5% crystal violet/10% ethanol). The resulting colonies were enumerated by the colony counting macro in Image) software, SKOV3-ovarian cancer cell lines. Micrographs of the stained plated are shown in FIG. 14A. Colony counts are indicated on the right of the micrograph (n=8).

Data in FIG. 14B demonstrates that there are distinct effects on tumor spheroids after coculture of different cancer cell lines with unprimed MSCs, MSC1, or MSC2. Tumor spheroids were formed by culturing tumor cells (2000 cells/well) mixed without any other cells (- -) or with hMSCs, MSC1, or MSC2 (20 cells/well) at a ratio of 10 cancer cells per 1 MSC and plated over 1.5% agarose in 96-well plates in growth medium supplemented with 10% FBS. Cultures were grown for 14 days at 37° C. in a humidified atmosphere of 5% carbon dioxide balance air. Growth medium was changed every 3-4 days. Micrographs shown in FIG. 14B represent 20× magnified field of the 96-well plate. Cancer cell lines used are OVCAR-human ovarian cancer, SKOV3-human ovarian cancer cell lines, and MOSEC-murine ovarian surface epithelium carcinoma cells. Data indicates distinct effects by MSC1 and MSC2 on cancer cell growth and spread.

### Example 19

### Evidence for MSC1 and MSC2 Distinct Treatment Effects of Painful Diabetic Peripheral Neuropathy Mouse Model: Heat Hyperalgesia

The following data provide evidence for MSC2 having a therapeutic effect in streptozotocin (STZ)-induced diabetic mice with painful diebetic peripheral neuropathy (pDPN). Although the cause of pDPN is multi-factorial, it has been connected to inflammation, and recent studies have identified an association of inflammation and inflammatory disease with the injured nervous system. In particular, substances released from peripheral terminals of small diameter primary afferent fibers and from sympathetic postganglionic nerve terminals that trigger acute inflammation (e.g., vasodilatation and plasma extravasation), as well as regulate neuronal tissue injury have been identified in pre-clinical inflammatory disease animal models (Basbaum, A. I. & Levine, J. D. The contribution of the nervous system to inflammation and inflammatory disease. Can J Physiol Pharmacol 69, 647-51 (1991), the disclosure of which is incorporated herein by reference). Moreover, glucose is in itself pro-inflammatory and increases the levels of acute-phase inflammatory markers, including tumor necrosis factor-alpha (TNF-α), interleukin (IL)-6 and C-reactive protein (CRP). These acute phase inflammatory markers are associated with insulin resistance and metabolic syndrome, suggesting a role for chronic low-grade inflammation in diabetes. See, e.g., Bailey, C. J., “Treating insulin resistance: future prospects”, Diab. Vasc. Dis. Res., 4: 20-31 (2007); Niehoff, A. G. et al., “C-reactive protein is independently associated with glucose but not with insulin resistance in healthy men”, Diabetes Care, 30:1627-9 (2007); and Sjoholm, A. & Nystrom, T., “Endothelial inflammation in insulin resistance”, Lancet, 365: 610-2 (2005) (the disclosures of all of which are incorporated herein by reference. Therefore, therapy focused on decreasing the production of pro-inflammatory mediators in diabetics appears necessary.

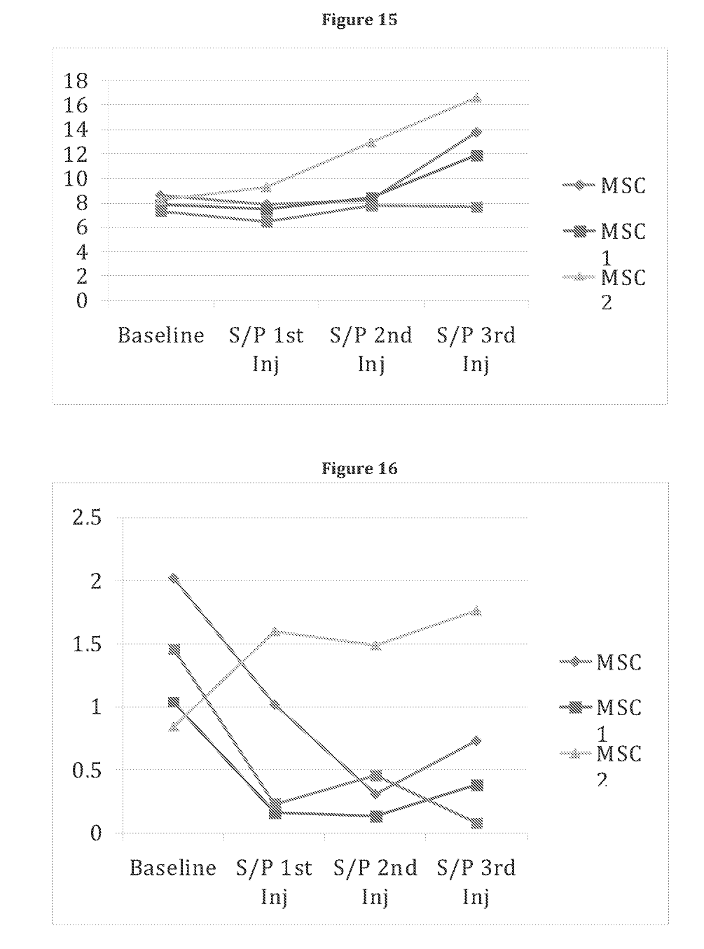

Hyperalgesia is an enhanced response to noxious stimulation, and is associated with painful diabetic peripheral neuropathy. In the assay results shown in FIG. 15, diabetic mice were evaluated for their tolerance (measured in seconds before paw withdrawal) to heat applied to a paw. Streptozotocin (STZ)-induced diabetic mice were procured from commercial sources (Jackson Laboratory, Bar Harbor, Me.). Blood glucose levels and animal weights were measured by standard methods. A month post STZ-injection, mice received intraperitonealy (IP) 0.5×10⁶ cells of MSCs, MSC1, MSC2, or HBSS vehicle for a total of 3 times in 10-day intervals. In some instances, human MSCs, MSC1 or MSC2 (1×10⁶ cells/mouse) were delivered 3-times IP at monthly intervals.

Established behavioral assays to evaluate hyperalgesia (and allodynia) were conducted one day prior to each MSC therapy, as well as prior to sacrifice. Mice were evaluated for heat hyperalgesia using Hargraeve's method the day before MSC delivery or sacrifice. Data are representative of triplicate measurements in two independent experiments. Inflammatory factors and immune cell changes were measured as before to characterize the treatment effects on inflammation (n=30).

At baseline, there were no differences in the number of seconds it took before paw withdrawal when a heat lamp was applied to the plantar surface of their feet. After treatment one, no significant differences were noted. However, after both the second and third injections, the mice treated with MSC2 differed significantly from the other three treatments in their ability to withstand the heat lamp. An increase in time indicates an improvement in the mouse's peripheral neuropathy symptoms. Data in FIG. 15 demonstrate that treatment with MSC2 improves diabetic peripheral neuropathy heat hyperalgesia over baseline controls, MSC, and MSC1.

### Example 20

### Evidence for MSC1 and MSC2 Distinct Treatment Effects of Painful Diabetic Peripheral Neuropathy Mouse Model: Mechanical Allodynia

Mechanical allodynia is pain in response to light touch or pressure. It is also prevalent in diabetic peripheral neuropathy. FIG. 16 shows the effect on mechanical allodynia of MSC-treatments of streptozotocin (STZ)-induced diabetic mice. Microfilaments of various weights (gm) were applied to the hindpaw. Mice with mechanical allodynia are not capable of withstanding increased pressure from the microfilaments.

STZ-induced diabetic mice were acquired from commercial sources (JAX Labs). Human MSCs, MSC1 or MSC2 (1×10⁶ cells/mouse) were delivered 3-times IP at monthly intervals. Mice were evaluated for mechanical allodynia by the von Frey microfilament method the day before MSC delivery or sacrifice. Data are representative of triplicate measurements in two independent experiments.

At baseline, there were no statistically significant differences between the four treatments. However, after each injection, the mice that received MSC2 had a statistically significant greater ability to tolerate increases in weight of the microfilaments when compared to the other three treatments. This ability to withstand the increases is an indication of improvement in mechanical allodynia. Data provided in FIG. 16 demonstrate that treatment with MSC2 improves DPN mechanical allodynia over baseline controls, MSC, and MSC1.

### Example 21

### Evidence for MSC1 and MSC2 Distinct Treatment Effects of Painful Diabetic Peripheral Neuropathy Mouse Model: Serum Cytokine/Chemokine Secretion

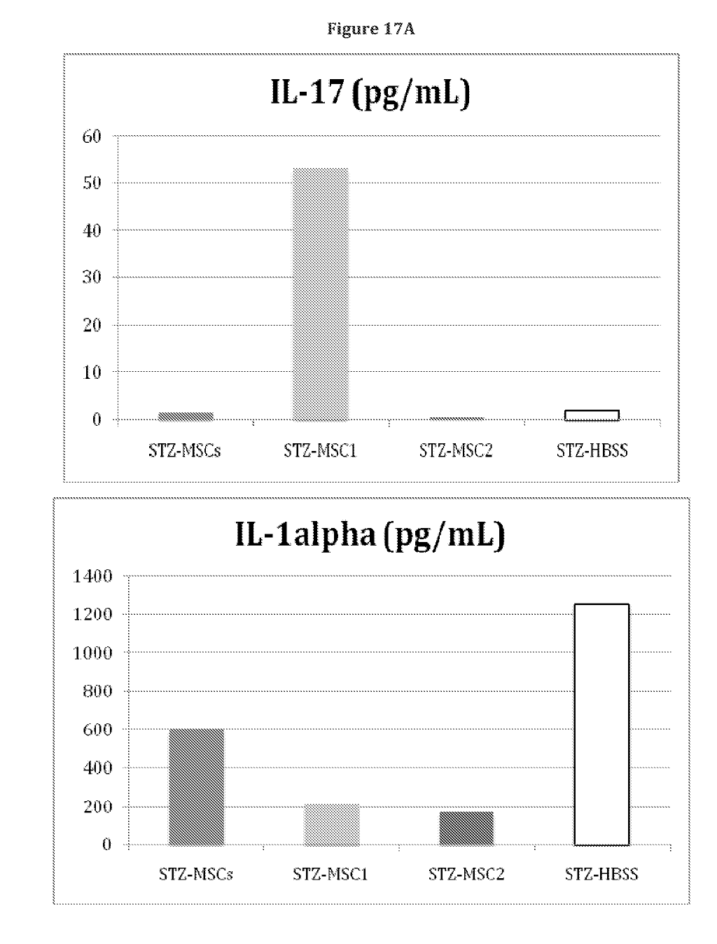

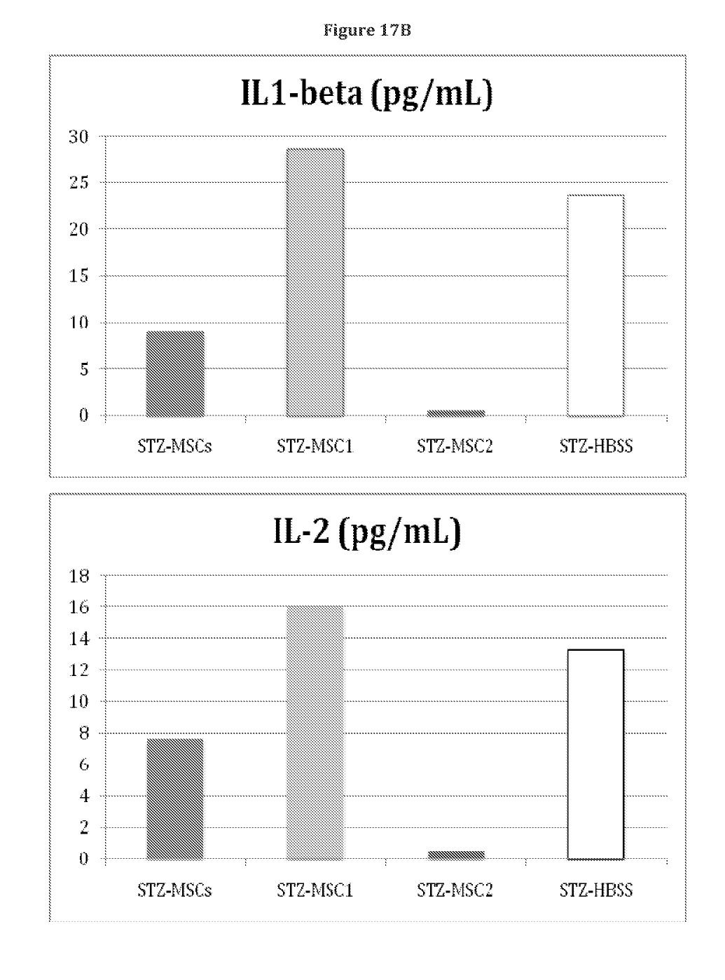

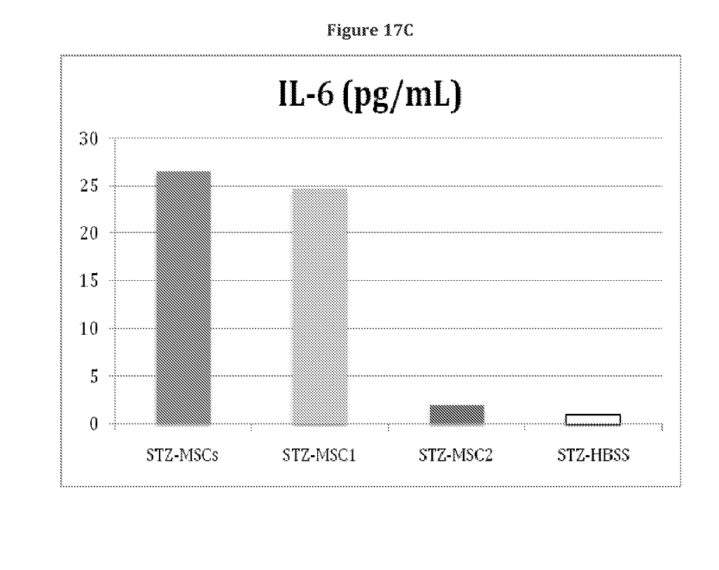

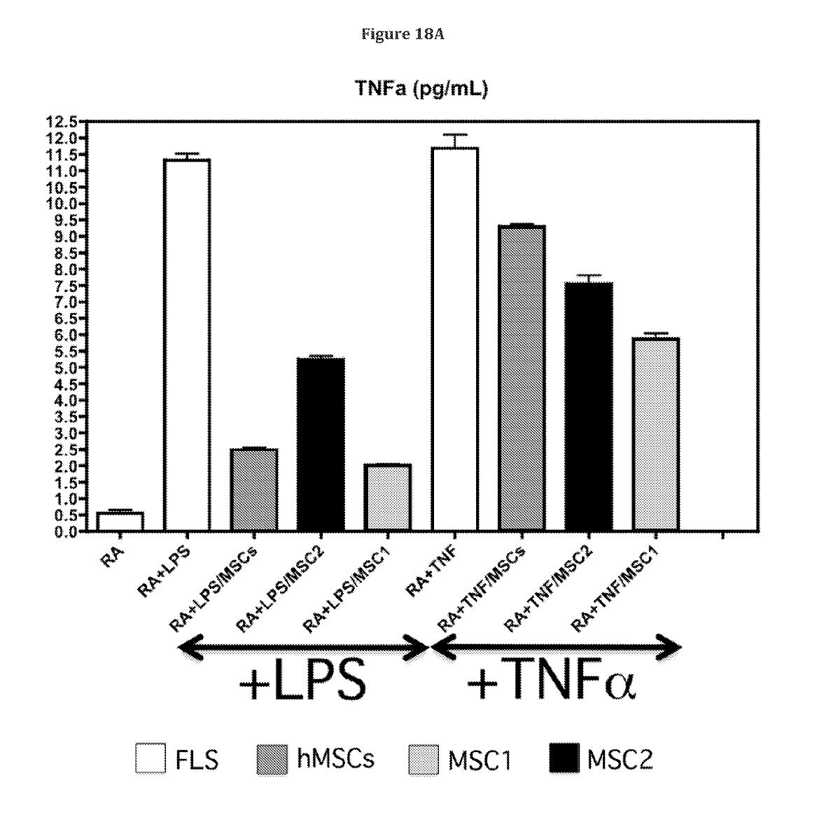

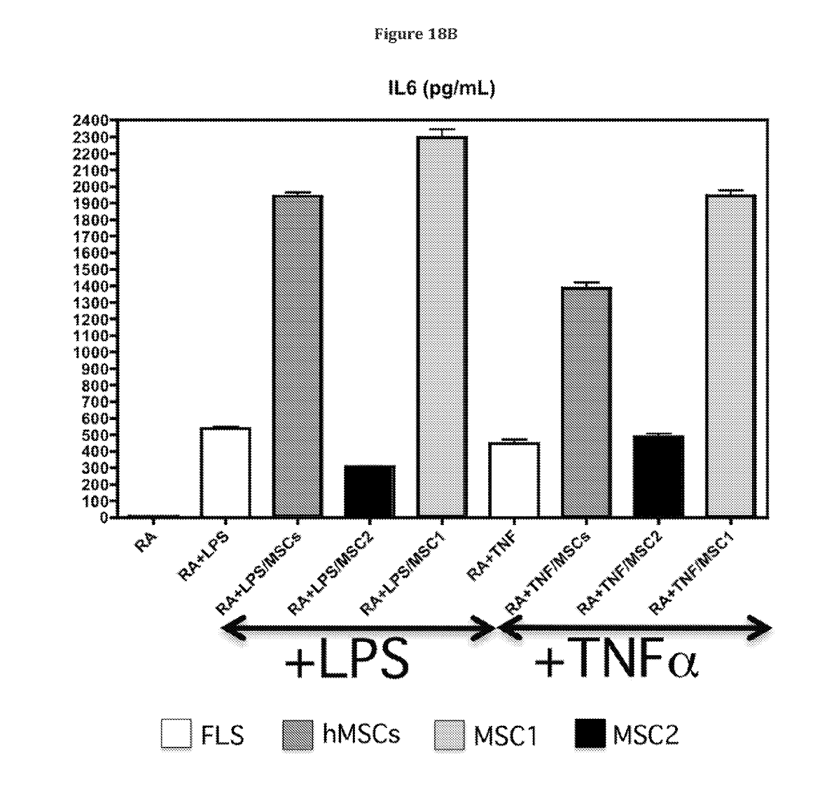

Data in FIG. 17 show cytokine/chemokine secretion in serum of streptozotocin (STZ)-induced diabetic mice. The graphs in FIG. 17 demonstrate that treatment with MSC2 reduced the secretion of pro-inflammatory factors and stimulated the secretion of anti-inflammatory ones. STZ-induced diabetic mice were acquired from commercial sources (JAX Labs). Human MSCs, MSC1 or MSC2 (1×10⁶ cells/mouse) were delivered 3-times IP at monthly intervals. Mice were sacrificed 19 days after the last MSC treatment. Mouse serum was derived from blood taken by cardiac puncture. Serum samples were analyzed by Bio-Plex Cytokine Assays (murine 32-plex; Bio-Rad, Hercules, Calif.) following the manufacturer's instructions. Data are presented after analyses as pg/mL. Data are representative of triplicate measurements in two independent experiments.

Data in FIGS. 17A, 17B, and 17C show that MSC2 reduced secretion of pro-inflammatory cytokines IL-1 alpha (interleukin), IL-1 beta, IL-2, IL-6, and IL-17 compared to the serum from mice of the other three treatments (MSCs, MSC1, and Hank's Balanced Salt Solution HBSS). While not wishing to be bound by a particular theory, these lower levels of pro-inflammatory cytokines are a contributing factor in the improvements of mechanical allodynia and hyperalgesia seen in the MSC2 therapy.

### Example 22

### RA-MSC Allogeneic Co-Culture Cytokine/Chemokine Secretion

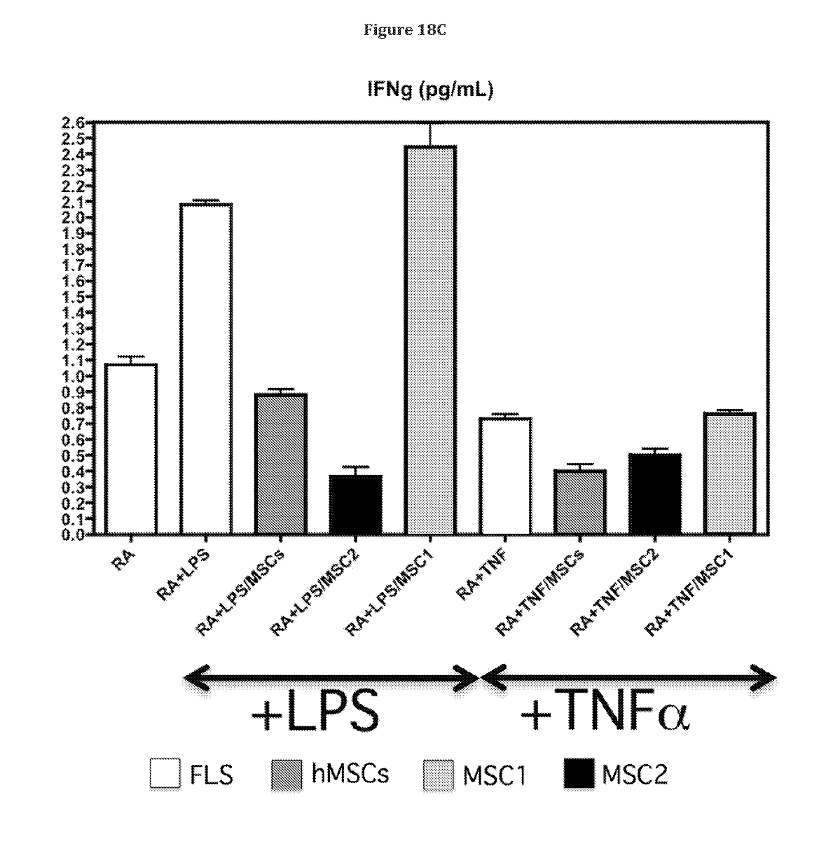

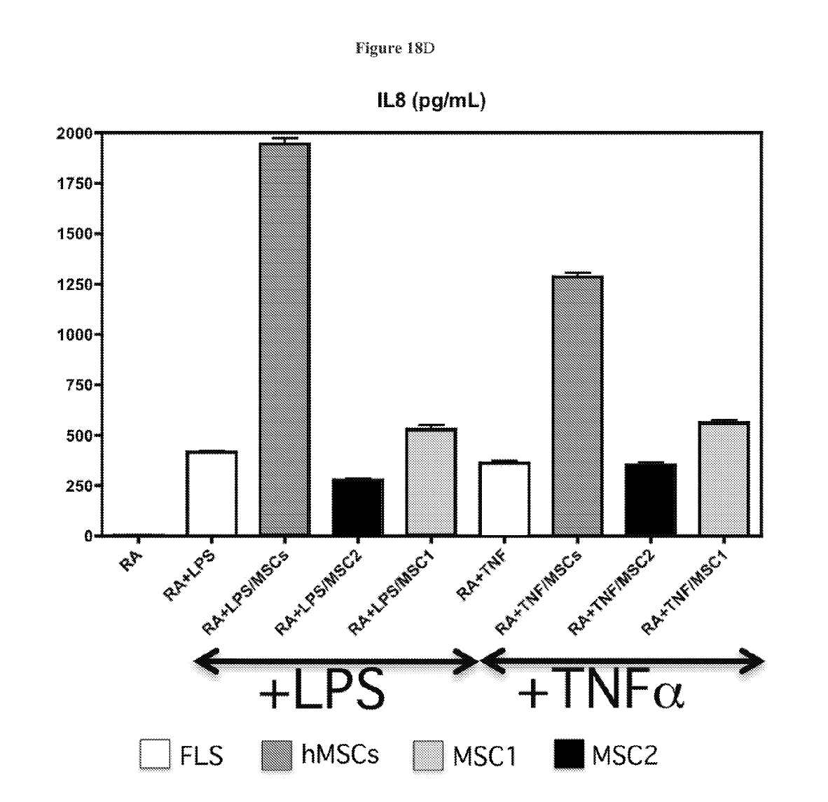

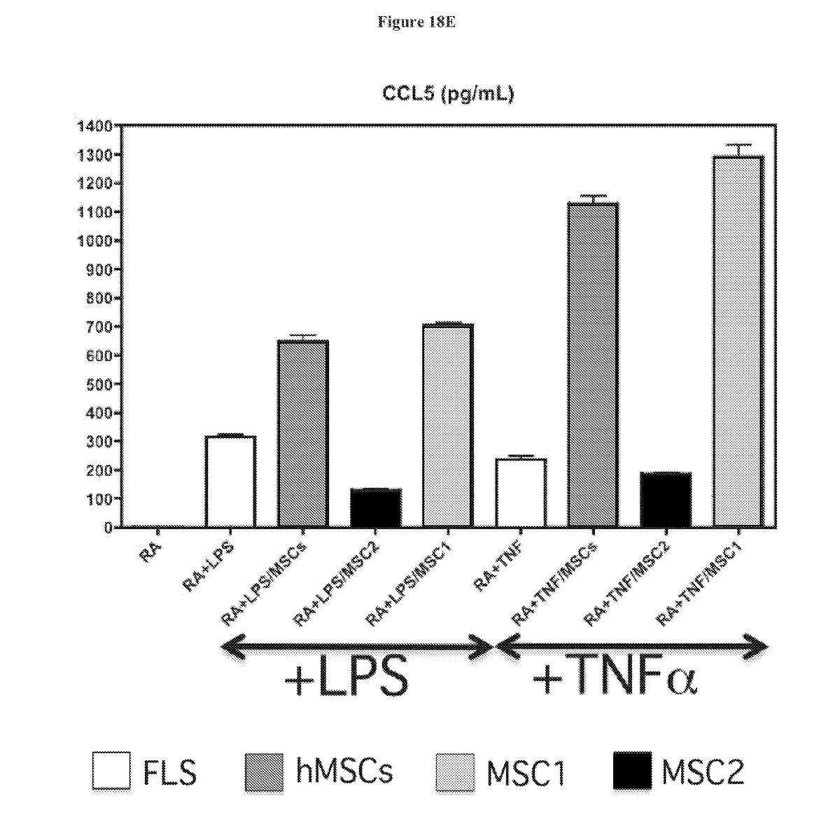

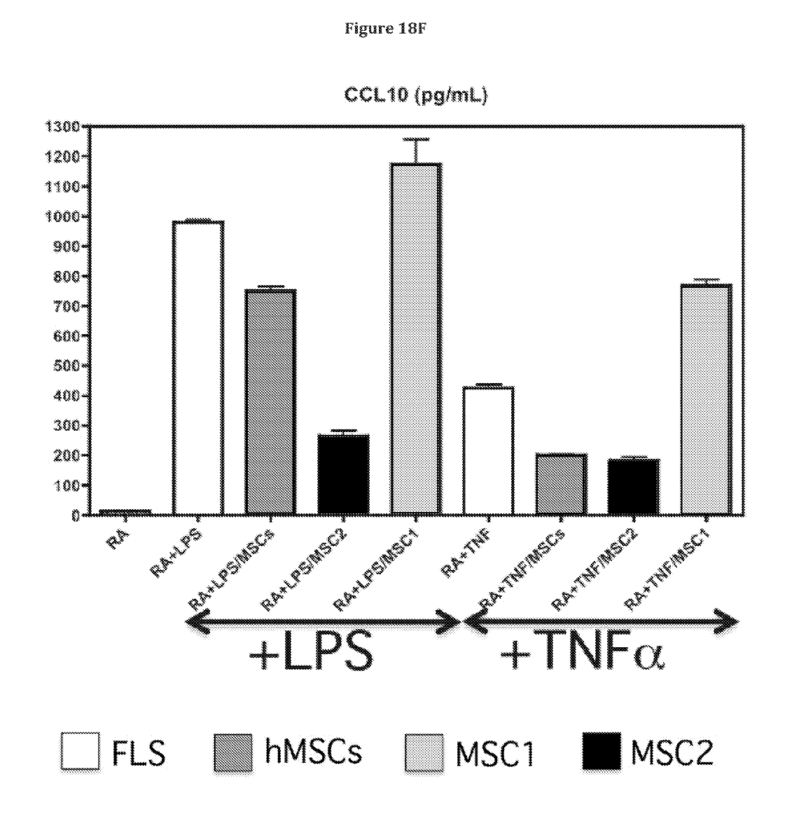

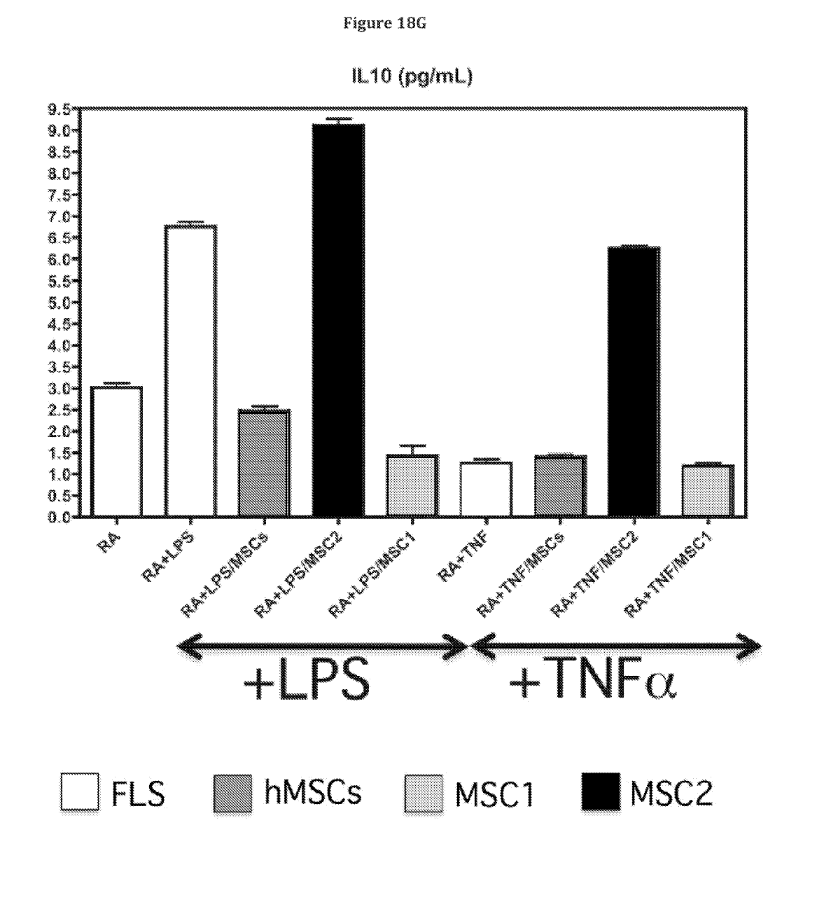

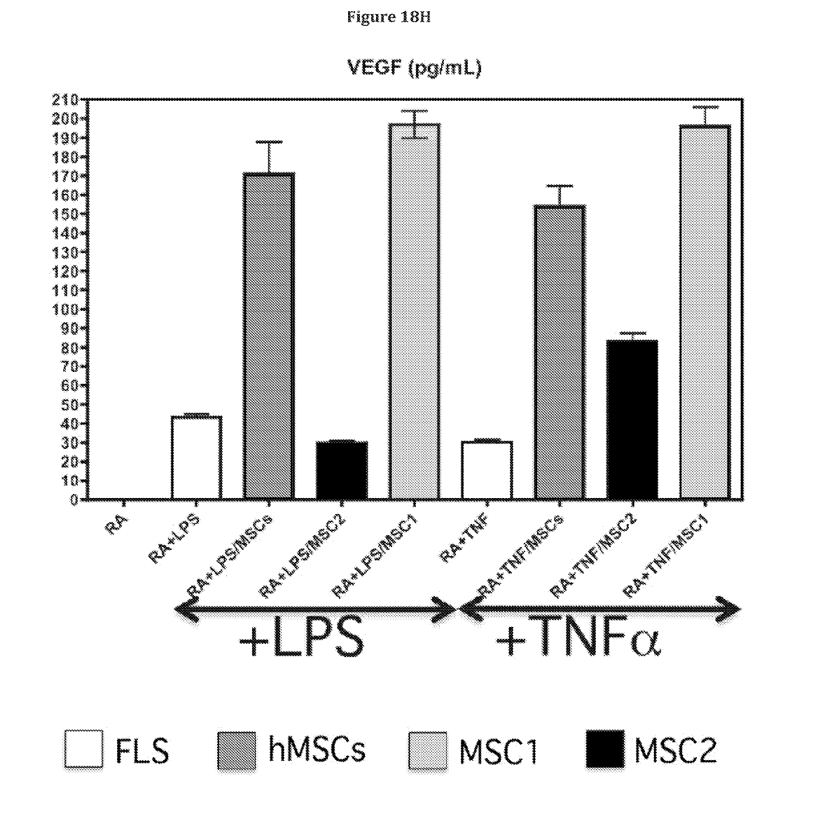

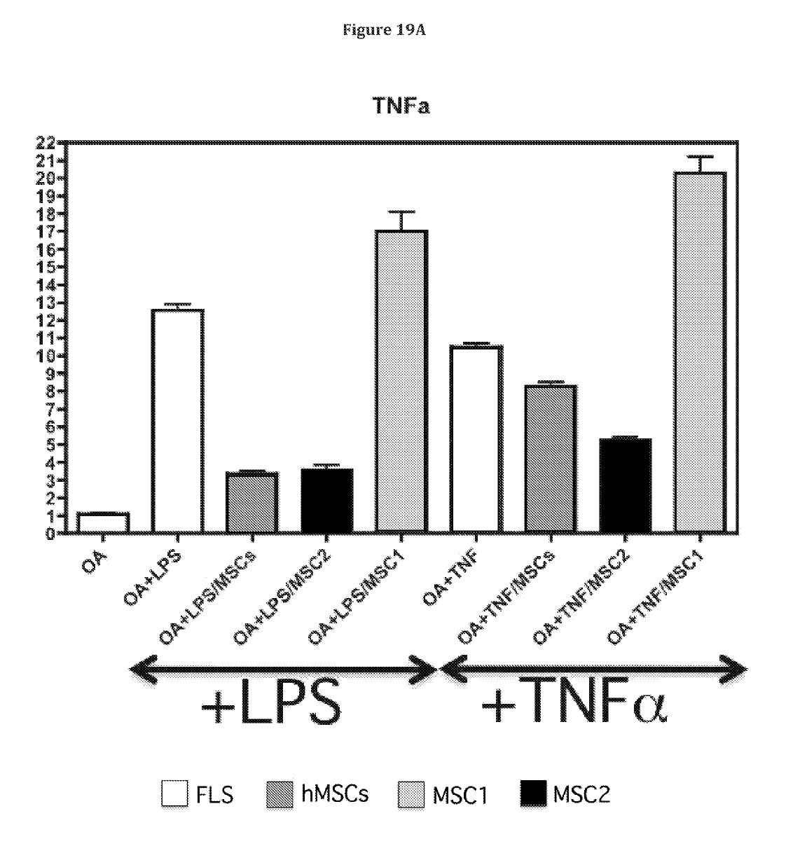

The following study uses co-cultures with human fibroblast-like synoviocytes (FLS) derived from rheumatoid arthritis (RA) patients. This data, as illustrated in FIG. 18 supports that MSC1 are pro-inflammatory whereas MSC2 are anti-inflammatory when co-cultured with RA FLS.

RA FLS (#1624 p5) were plated to 70% confluence and stimulated with 100 ng/mL LPS or 20 ng/mL TNF-alpha or left unstimulated for 24 hrs. Human MSCs were pre-treated for 1 hr with Toll like receptor agonists (LPS for MSC1 or poly(I:C) for MSC2) or not (hMSCs), washed, and then harvested. Approximately, 2×10⁵ FLS cells were mixed with 1×10⁵ MSC cells, and plated for an additional 48 hrs prior to harvesting the spent medium and analysis with Bio-Plex® cytokine assays (Human Group I & II, Bio-Rad®, Hercules, Calif.) following the manufacturer's instructions. Data are presented in FIG. 18 after analyses as pg/mL. Data in FIG. 18 are representative of triplicate measurements in two independent experiments.

### Example 23

### OA-MSC Allogeneic Co-Culture Cytokine/Chemokine Secretion

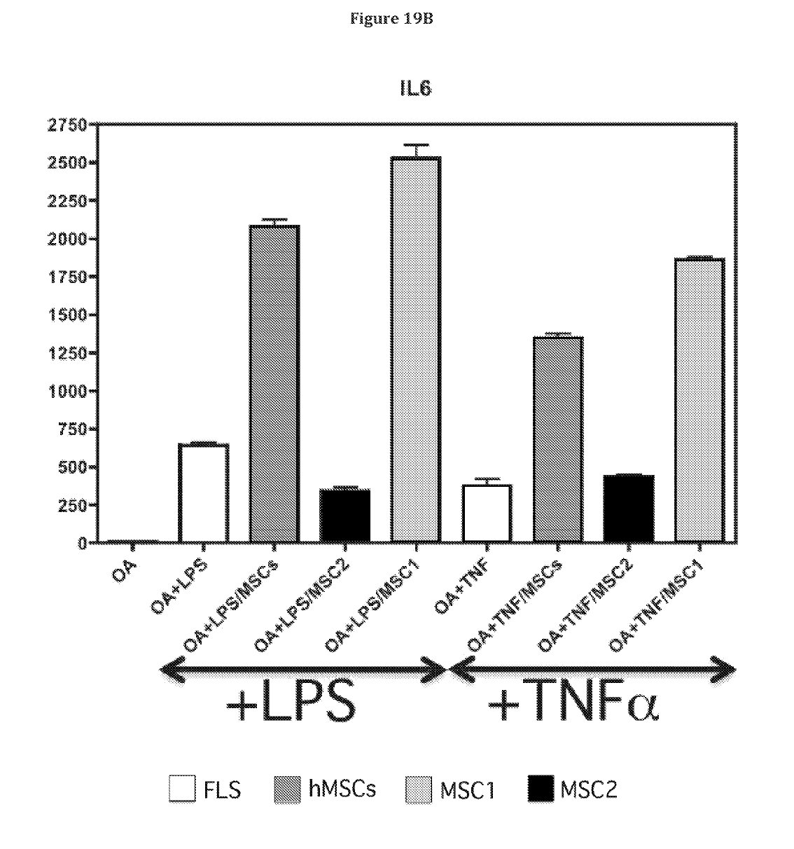

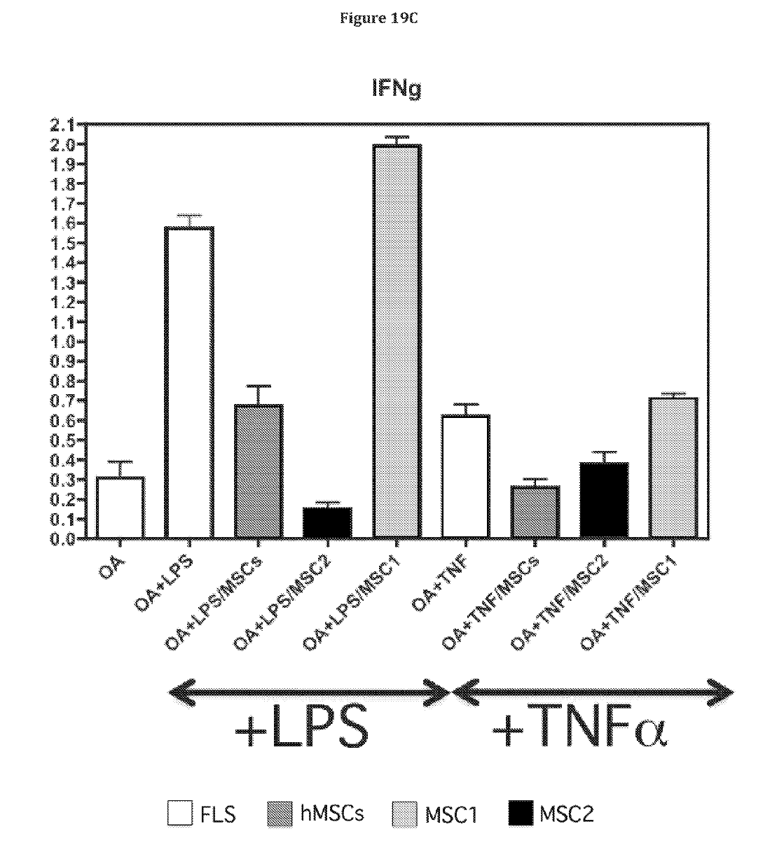

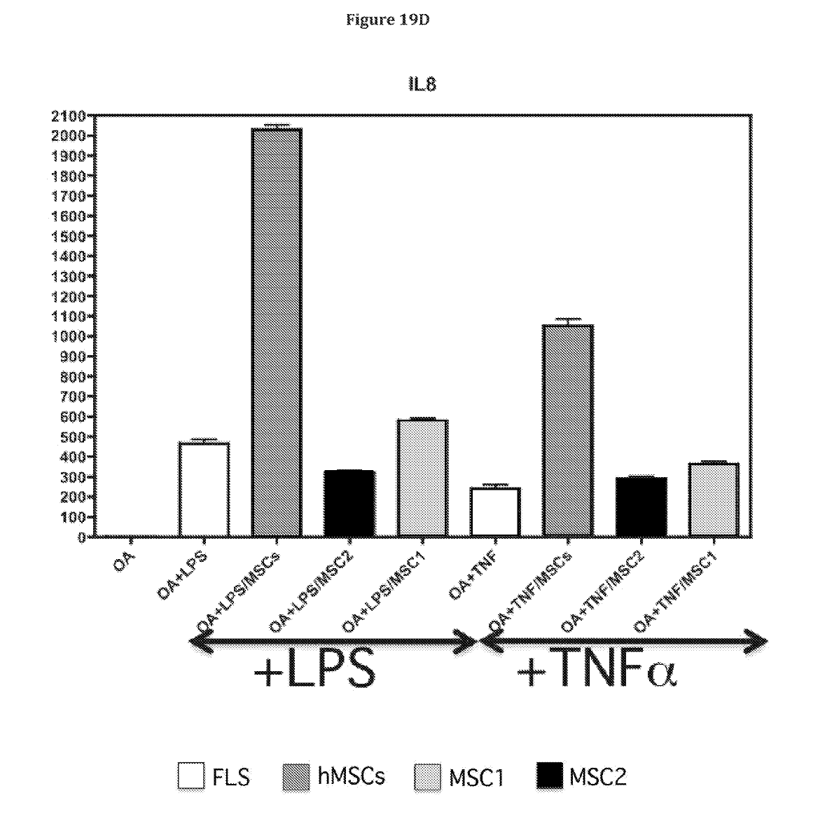

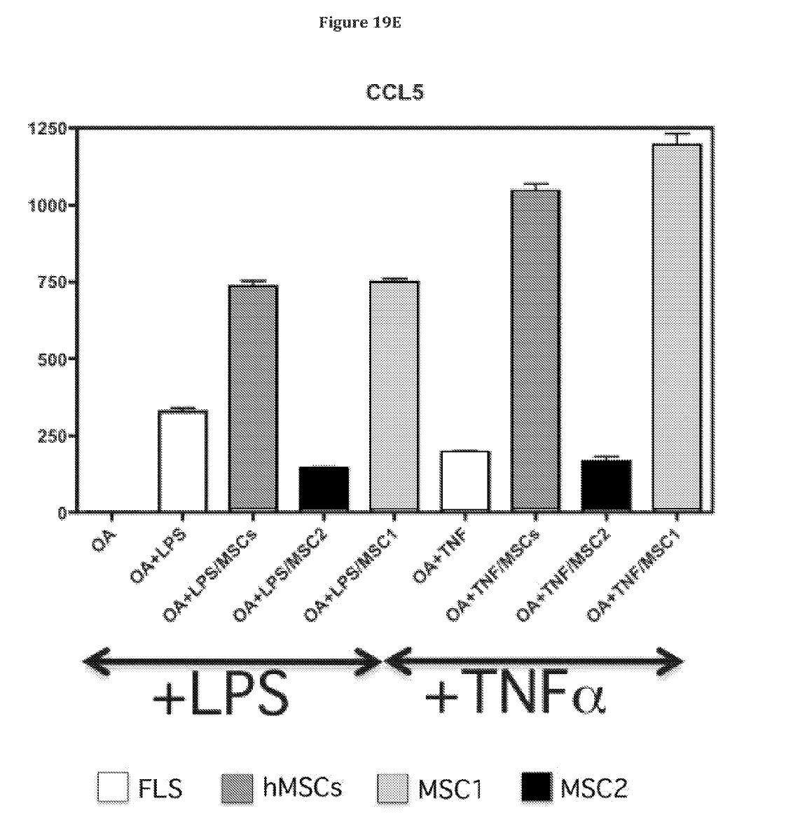

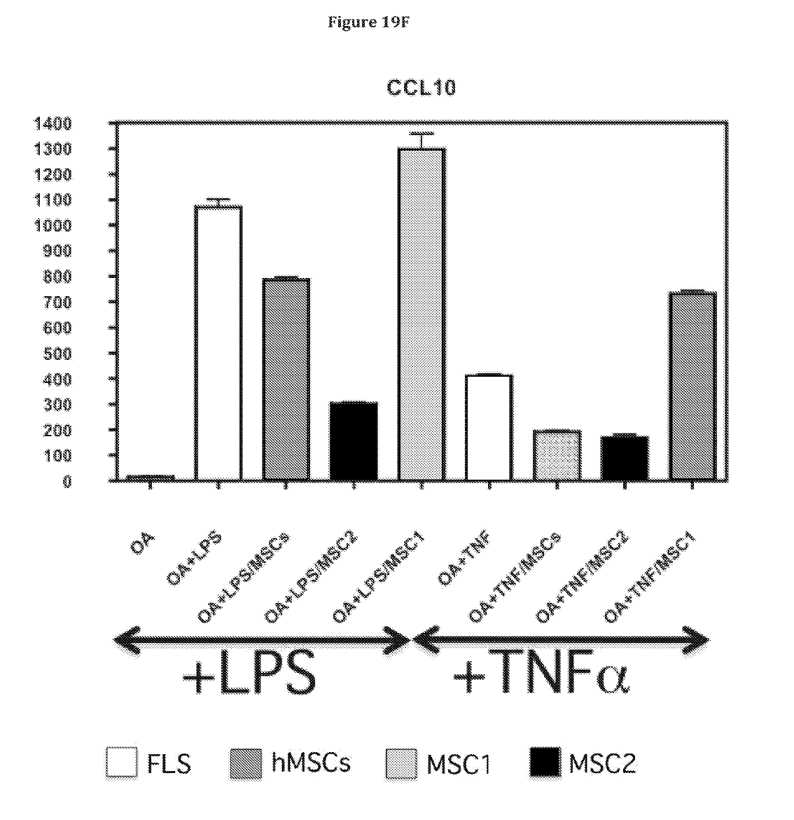

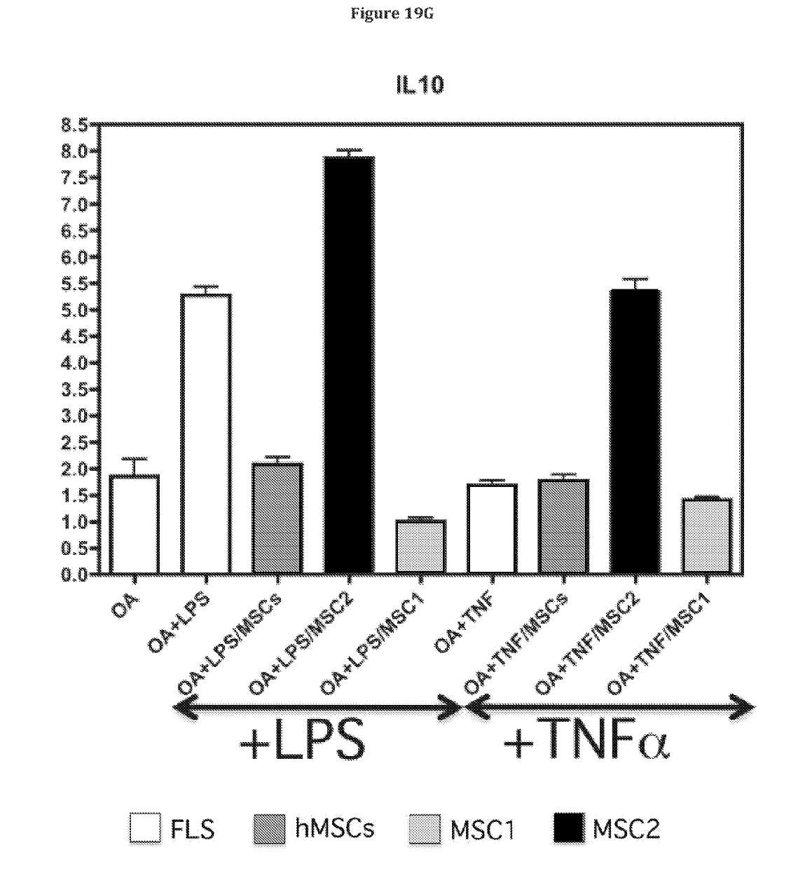

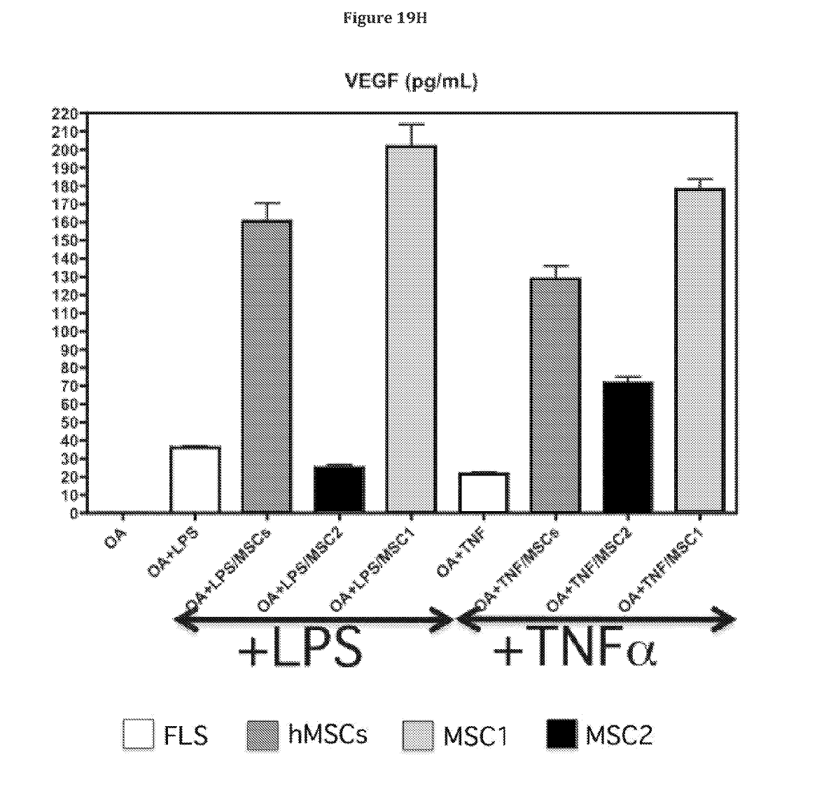

The following study uses co-cultures with human fibroblast-like synoviocytes (FLS) derived from osteoarthritis (OA) patients. This data, as illustrated in FIG. 19 supports that MSC1 are pro-inflammatory whereas MSC2 are anti-inflammatory when co-cultured with OA FLS.

OA FLS (#1561 p5) were plated to 70% confluence and stimulated with 100 ng/mL LPS or 20 ng/mL TNF-alpha or left unstimulated for 24 hrs. Human MSCs were pre-treated for 1 hr with Toll like receptor agonists (LPS for MSC1 or poly(I:C) for MSC2) or not (hMSCs), washed, and then harvested. Approximately, 2×10⁵ FLS cells were mixed with 1×10⁵ MSC cells, and plated for an additional 48 hrs prior to harvesting the spent medium and analysis with Bio-Plex® cytokine assays (Human Group I & II, Bio-Rad®, Hercules, Calif.) following the manufacturer's instructions. Data are presented in FIG. 19 after analyses as pg/mL. Data in FIG. 19 are representative of triplicate measurements in two independent experiments.

### Example 24

### Quantitative PCR (qPCR)-RNA Expression of Allogenic Co-Cultures of Human Fibroblast-Like Synoviocytes (FLS) Derived from Rheumatoid Arthritis (RA) Patients with Varying MSC

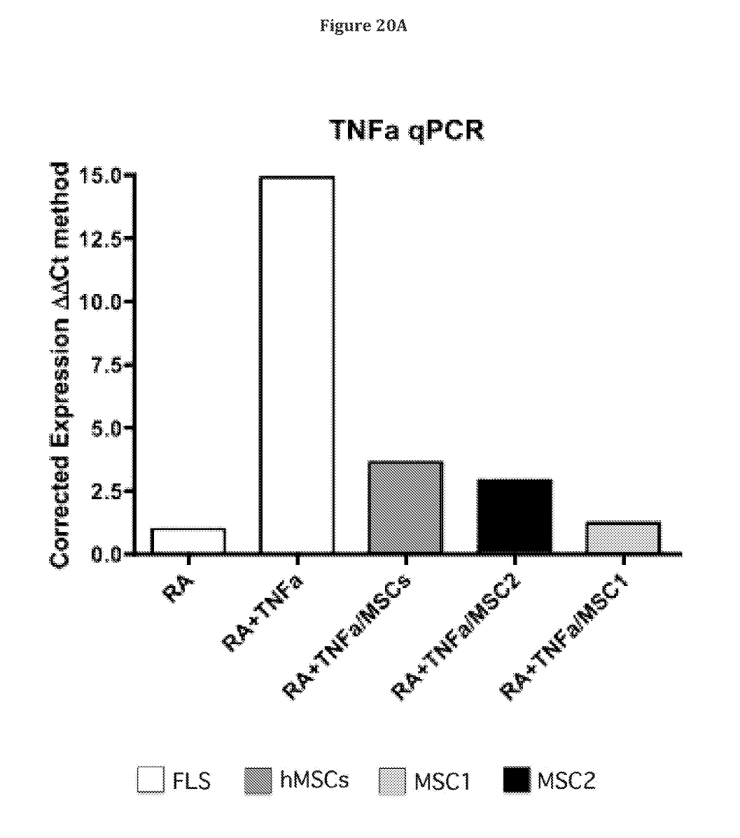

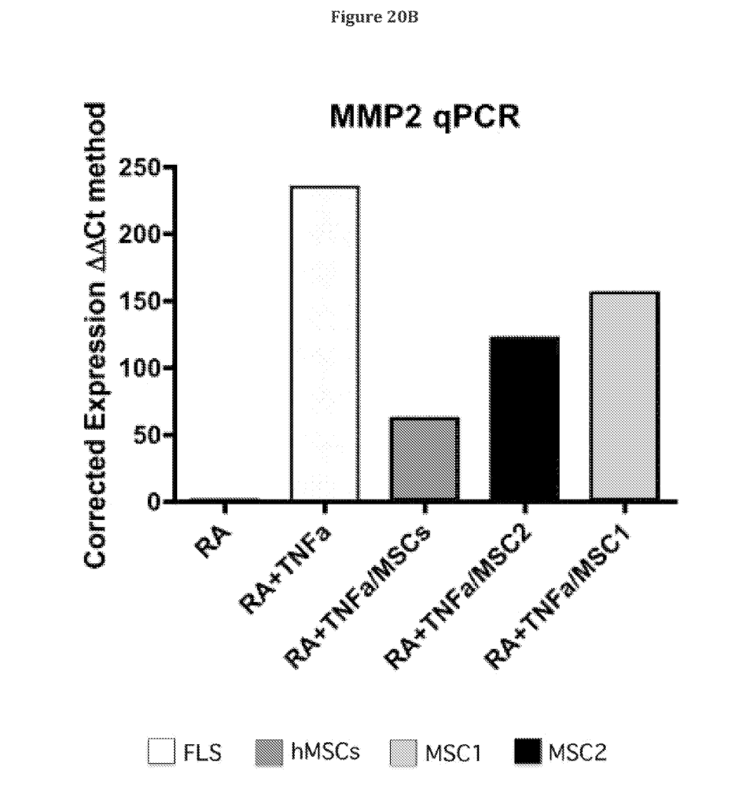

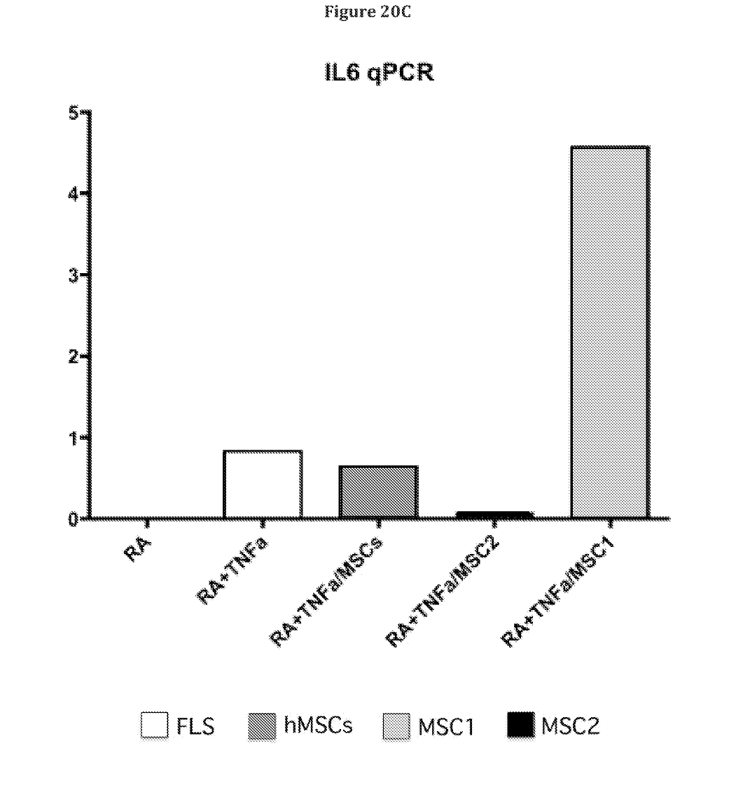

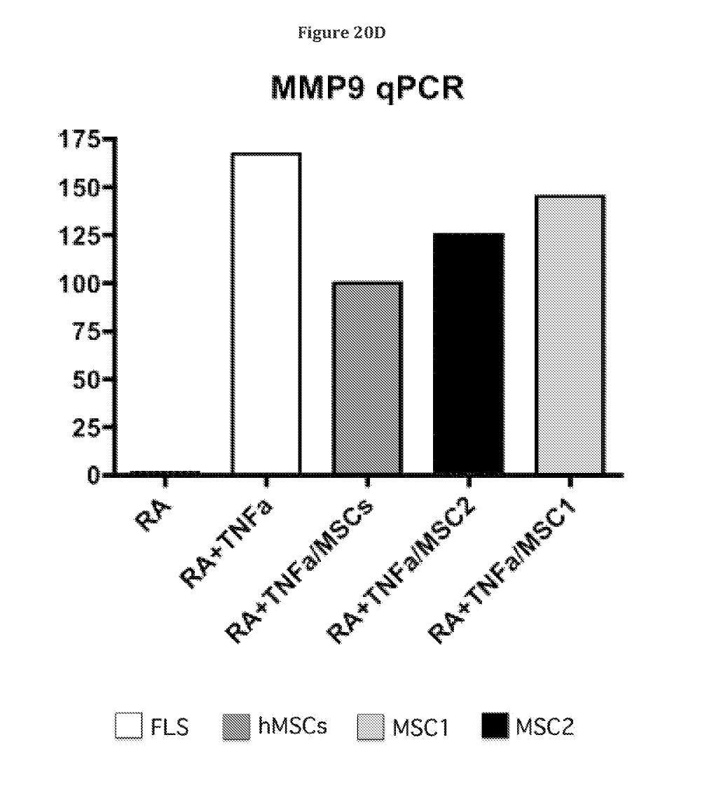

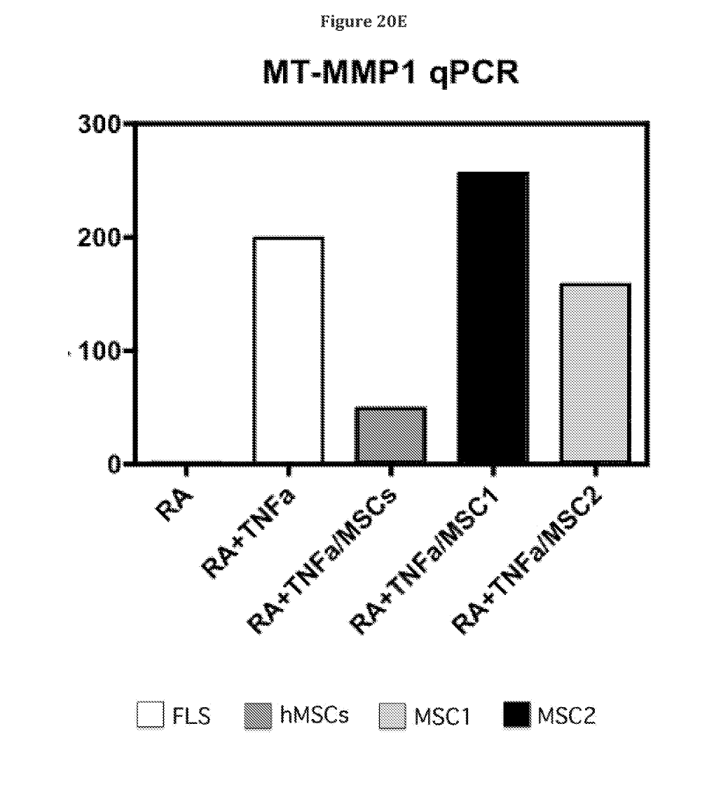

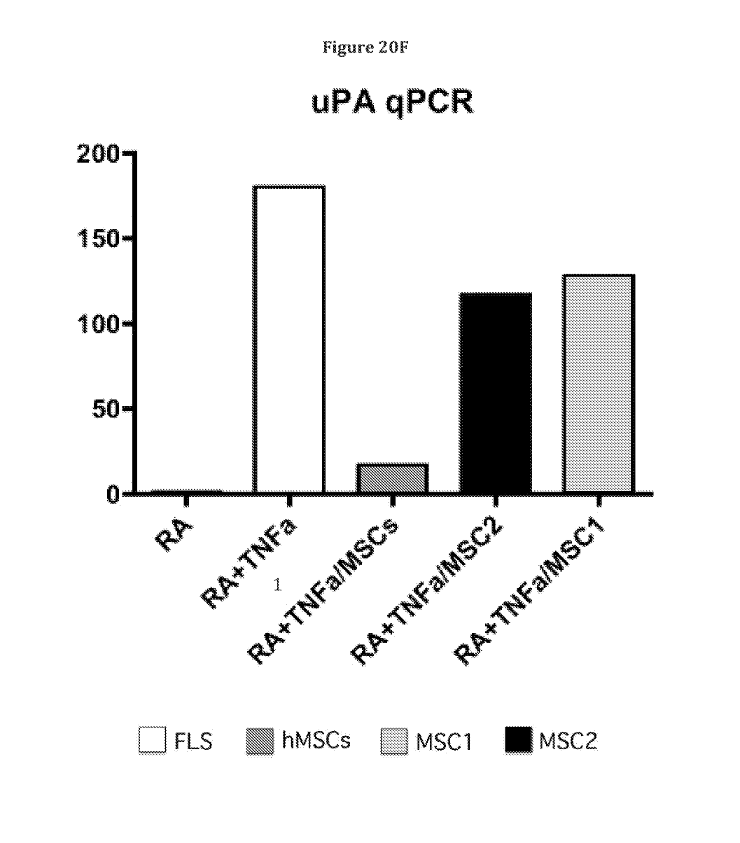

FIG. 20 shows results from qPCR-RNA expression assays of allogenic co-cultures of human fibroblast-like synoviocytes (FLS) derived from rheumatoid arthritis (RA) patients with varying MSC, as determined by the ΔΔ cumulative threshold method (C(t)) with 18srRNA as internal housekeeping target gene. RNA along with microRNAs were extracted by miRNAeasy kit (Qiagen©) from allogeneic RA-FLS:MSC co-culture (2:1) cells grown in 24-well plates. Quantitative real time primers for human IL6, TNF-alpha, MMP2, MMP9, MT-MMP1 and uPA were commercially obtained (Qiagen©) and primer efficiencies were verified as standard prior to qPCR. All primer efficiencies were greater than 100%. QuantiFast RT-PCR SYBR® Green master mixes, RNA, and primers were combined and analyzed in a BioRad® CFX™ Cycler. Normalized expression for each RNA was determined by the ΔΔCumulative threshold (C(t)) method with 18srRNA as the internal housekeeping target gene. Samples were run in triplicate for two independent experiments.

FIGS. 20A, 20B, 20C, 20D, 20E, and 20F show graphs of normalized RNA expression using TNF-alpha, MMP2, IL-6, MMP9, MT-MMP1, and uPA primers, respectively.

### Example 25

### Collagen I Migration/Invasion Assays of FLS-MSC Allogeneic Co-Cultures

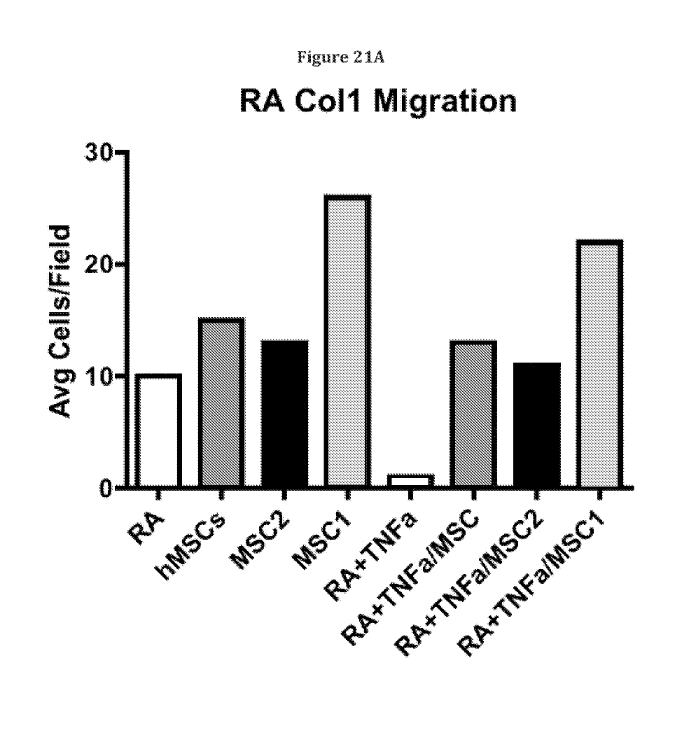

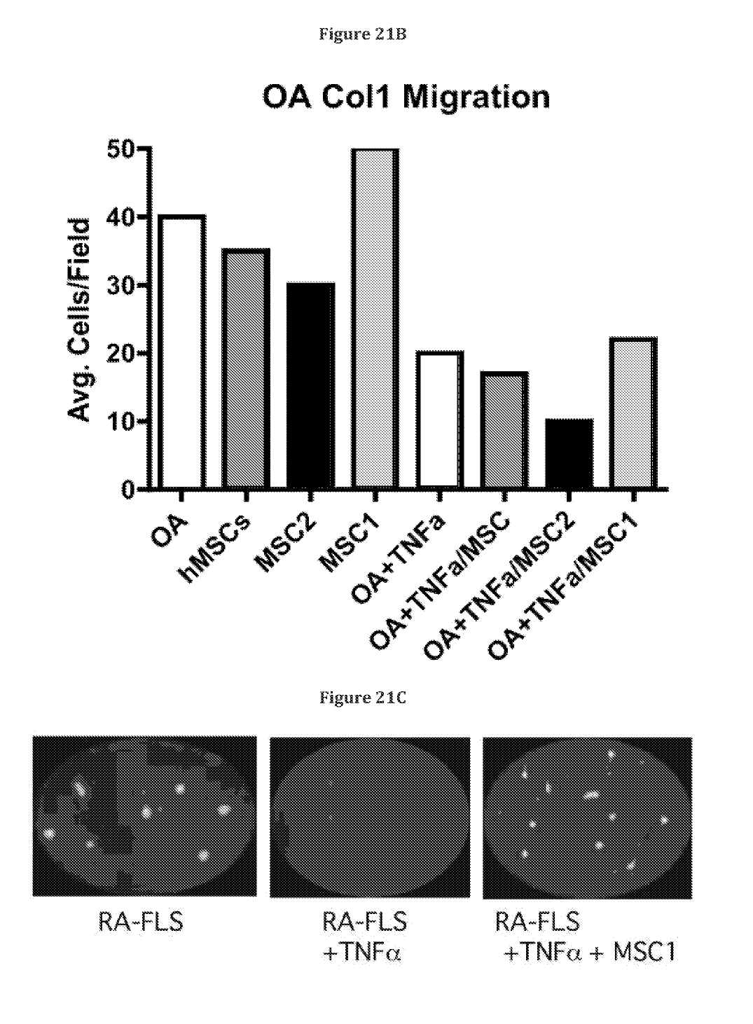

FIG. 21 shows results from a collagen I migration/invasion assay of allogenic co-cultures of human fibroblast-like synoviocytes (FLS) derived from rheumatoid arthritis (RA) patients of osteoarthritis (OA) patients with varying MSC.

3 micromolar Falcon Fluoroblok™ transwell inserts were coated with 2 mg/mL rat tail collagen I (Sigma-Aldrich®) and left to polymerize overnight at 4° C. FLS were plated and stimulated with TNF-alpha (20 ng/mL) overnight prior to harvest in serum-free medium. Next day, MSCs were stimulated with Toll like receptor agonists (LPS for MSC1 or poly(I:C) for MSC2) or not (MSC) for 1 hr, washed, and harvested in serum-free medium as before. Approximately, 2×10⁵ FLS cells were mixed with 1×10⁵ of either MSC, MSC1, or MSC2 cells and loaded in serum-free medium on top of collagen I coated inserts. Inserts were placed over wells with 20% serum containing-medium (as chemoattractant) and allowed to incubate for 16 hours in a humified CO₂ incubator. Inserts were transferred to calcein AM-HBSS wells and allowed to incubate for 1 hour at 37° C.

FIGS. 21A & 21B show graphs of average cell numbers per viewing field. Data presented are the average count of 3 fields per sample. Data are representative of duplicates in two independent experiments. FIG. 21C is illustration of migrating and invading cells visualized on an inverted fluorescence microscope (200×, Olympus®).

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. An isolated, stimulated mesenchymal stem cell, wherein the stimulated mesenchymal stem cell demonstrates, versus a mesenchymal cell that is not stimulated:
elevated secretion of IL4, IL6, and IL8, reduced secretion of TGFβ1, and increased expression of Jagged 1, MIR 155, and Bic; or
elevated secretion of IL4, IP10, RANTES, IL1RA, PGE2, and SMAD7,
reduced expression of TGFβ1, TGFβ3, Jagged 1, MIR155, and Bic, and increased indoleamine 2,3-dioxygenase activity.

2. The cell of claim 1, wherein the cell is stimulated with a Toll-like receptor ligand.

3. The cell of claim 2, wherein the Toll-like receptor ligand is selected from the group consisting of IL4, IL13, poly(A:U), poly(I:C), and combinations thereof, and aminoalkyl glucosaminide 4-phosphates, interferons, TNF-alpha, GM-CSF, lipopolysaccharide (LPS), and combinations thereof.

4. The cell of claim 3, wherein cell is incubated with the Toll-like receptor ligand for up to 2 hours, then removed.

5. The cell of claim 4, wherein the incubation is for up to 60 minutes.

6. The cell of claim 5, wherein the Toll-like receptor ligand is poly(I:C).

7. The cell of claim 5, wherein the Toll-like receptor ligand is LPS.

8. An isolated mesenchymal stem cell stimulated with at least one TLR3 ligand, wherein the stimulated mesenchymal stem cell exhibits,
elevated secretion of IL4, IL 10, CXCL5 (RANTES), CXCL10 (IP10), and PGE2;
reduced expression of TGFβ1, TGFβ3, Jagged 1, MIR155, and Bic; and
increased indoleamine 2,3-dioxygenase activity;
in comparison to an isolated mesenchymal cell that is not stimulated with the at least one TLR3 ligand.

9. The stimulated stem cell of claim 8, wherein the Toll-like receptor 3 ligand is poly(I:C).

10. The stimulated stem cell of claim 8, wherein the mesenchymal stem cell is incubated with a Toll-like receptor 3 ligand for up to 60 minutes.

11. The stimulated stem cell of claim 8, wherein the mesenchymal cell that is not stimulated with Toll-like receptor 3 ligand is a mesenchymal cell that is stimulated by at least one Toll-like receptor 4 ligand.

12. The TLR3-stimulated stem cell of claim 11, wherein said cellexhibits:
(i) increased secretion of fibronetin, and
(ii) decreased secretion of collagen,
in comparison to a mesenchymal cell that is stimulated with at east one TLR 4 ligand.

13. The TLR3-stimulated stem cell of claim 11, wherein the mesenchymal cell that is stimulated with the at least one TLR3 ligand exhibits inhibited chondrogenesis, osteogenesis, and adipogenesis, and wherein the mesenchymal cell that is stimulated with the at least one TLR4 ligand exhibits inhibited adipogenesis and stimulated osteogenesis.

14. An isolated mesenchymal stem cell stimulated with at least one TLR 4 ligand, wherein the stimulated mesenchymal stem cell exhibits:
elevated secretion of IL6 and IL8;
reduced secretion of TGFβ1; and
increased expression of Jagged 1, MIR155, and Bic;
in comparison to an isolated mesenchymal cell that is not stimulated with the at least one TLR4 ligand.

15. The stimulated mesenchymal stem cell of claim 14, wherein the Toll-like receptor 4 ligand is LPS.

16. The stimulated mesenchymal stem cell of claim 14, wherein the mesenchymal stem cell is incubated with a Toll-like receptor 4 ligand for up to 60 minutes.

17. The stimulated mesenchymal stem cell of claim 14, wherein the mesenchymal cell that is not stimulated with TLR4 ligand is a mesenchymal cell that is stimulated with at least one TLR 3 ligand.

18. The TLR3-stimulated mesenchymal stem cell of claim 17, wherein said cell exhibits:
(i) decreased secretion of fibronetin, and
(ii) increased secretion of collagen,
in comparison to a mesenchymal cell that is stimulated with the at least one TLR3 ligand.

19. The cell of claim 17, wherein the mesenchymal cell that is stimulated with the at least one TLR3 ligand exhibits inhibited chondrogenesis, osteogenesis, and adipogenesis, and wherein the mesenchymal cell that is stimulated with the at least one TLR4 ligand exhibits inhibited adipogenesis and stimulated osteogenesis.

20. A method for stimulating mesenchymal stem cells, comprising:
(a) isolating mesenchymal stem cells into a culture medium;
(b) incubating the mesenchymal stem cells of (a) for up to 2 hours with a Toll-like receptor ligand selected from the group consisting of IL4, IL13, poly(A:U), poly(I:C), and combinations thereof, and aminoalkyl glucosaminide 4-phosphates, interferons, TNF-alpha, GM-CSF, lipopolysaccharide (LPS), or combinations thereof;
(c) removing said Toll-like receptor ligand from the mesenchymal stem cells of (b); and
(d) optionally further incubating the mesenchymal stem cells of (c) thereby stimulating said mesenchymal stem cells.

21. The method of claim 20, wherein said incubation is from about 25 minutes to about 90 minutes.

22. The method of claim 21, wherein the incubation is for up to about 60 minutes.

23. The method of claim 20, wherein the Toll-like receptor ligand is poly (I:C) at a concentration of from about 0.5 μg/ml, to about 5 μg/mL of culture medium.

24. The method of claim 20, wherein the Toll-like receptor ligand is lipopolysaccharide at a concentration of from about 5 ng/mL to about 50 ng/mL of culture medium.

25. An isolated stimulated mesenchymal stem cell produced by a process comprising:
(a) isolating a mesenchymal stem cell into a culture medium;
(b) incubating the mesenchymal stern cell of (a) for up to 1 hour with a Toll-like receptor ligand selected from the group consisting of IL4, IL13, poly(A:U), poly(I:C), and combinations thereof, and aminoalkyl glucosaminide 4-phosphates, interferons, TNF-alpha, GM-CSF, lipopolysaccharide (LPS), and or combinations thereof;
(c) removing said Toll-like receptor ligand from the mesenchymal stem cell of (b); and
(d) optionally further incubating the mesenchymal stem cell of (c) thereby producing said stimulated mesenchymal stem cell.

26. The cell of claim 25, wherein said incubation is from about 25 minutes to about 90 minutes.

27. The cell of claim 26, wherein the incubation is for up to about 60 minutes.

28. The cell of claim 25, wherein the Toll-like receptor ligand is poly (I:C) at a concentration of from about 0.5 pg/ml to about 5 μg/mL of culture medium. 29, The cell of claim 25, wherein the T receptor ligand is poly (I:C) at a concentration of from about 5 ng/mL to about 50 ng/mL of culture medium.

30. The cell of claim 25, wherein the isolated mesenchymal stem cell is incubated with poly (I:C) at a concentration of about for about 60 minutes, and wherein the stimulated mesenchymal stem cell exhibits anti-inflammatory properties.

31. The cell of claim 25, wherein the isolated mesenchymal stem cell is incubated with LYS at a concentration of about 10 ng/mL for about 60 minutes, and wherein the stimulated mesenchymal stem cell exhibits pro-inflammatory properties.

32. An isolated mesenchymal stem cell for use in treating ovarian cancer, wherein said isolated mesenchymal stem cell is:
incubated with at least one TLR 4 ligand selected from the group consisting of aminoalkyl glucosaminide 4-phosphates, interferons, TNF-alpha, GM-CSF, lipopolysaccharide (LPS), and combinations thereof for up to 2 hours.

33. The isolated mesenchymal stem cell of claim 32, wherein the isolated mesenchymal stem cell is incubated with LPS for about 1 hour.

34. The isolated mesenchymal stem cell of claim 32, for use in reducing tumor growth in said ovarian cancer.

35. An isolated mesenchymal stern cell for use in treating diabetic peripheral neuropathy, wherein said isolated mesenchymal stern cell is:
incubated with at least one TLR 3 ligand selected from the group consisting of IL4, IL13, poly(A:U), poly(I:C), and combinations thereof for up to 2 hours.

36. The isolated mesenchymal stern cell of claim 35, for use in decreasing hyperalgesia associated with diabetic peripheral neuropathy.

37. The isolated mesenchymal stem cell of claim 35, for use in decreasing mechanical allodynia associated with diabetic peripheral neuropathy.

38. The isolated mesenchymal stem cell of claim 35, for use in lowering the serum level of at least one pro-inflammatory cytokine associated with diabetic peripheral neuropathy, wherein the pro-inflammatory cytokine is selected from the group consisting of IL-1 alpha, IL-1 beta, IL-2, IL-6, IL-17, and combinations thereof.

39. The isolated mesenchymal stem cell of claim 35, wherein the isolated mesenchymal stern cell is incubated with poly(I:C) for about 1 hour.

40. A stimulated, co-cultured mesenchymal stem cell produced by a process comprising:
(a) isolating a mesenchymal stern cell into a culture medium;
(b) incubating the isolated mesenchymal stem cell produced from step (a) for up to 1 hour with:
(i) a Toll-like receptor 3 ligand selected from the group consisting of IL4, IL13, poly(A:U), poly(I:C), and combinations thereof, or
(ii) a Toll-like receptor 4 ligand selected from the group consisting of aminoalkyl glucosaminide 4-phosphates, interferons, TNF-alpha, GM-CSF, lipopolysaccharide (LPS), and combinations thereof;
(c) isolating human fibroblast-like synoviocyte (FLS) cells derived from rheumatoid arthritis or osteoarthritis into a culture medium comprising TNF-alpha or lipopolysaccharide (LPS); and
(d) incubating the isolated mesenchymal stem cell produced from step (b) with the FLS cells produced from step (c);
thereby producing said stimulated, co-cultured mesenchymal stern cell.

41. The stimulated, co-cultured mesenchymal stem cell of claim 40, wherein the culture medium of step (c) comprises about 20 ng/mL of TNF-alpha or about 100 ng/mL of lipopolysaccharide (LPS).

42. The stimulated, co-cultured mesenchymal stem cell of claim 40, wherein the incubation of step (d) is from about 1 day to about 3 days.

43. An isolated mesenchymal stem cell for use in a method of treating acute lung injury, said method comprising delivering:
an isolated mesenchymal stem cell incubated with at least one Toll-like receptor 3 ligand selected from the group consisting of IL4, IL13, poly(A:U), poly(I:C), and combinations thereof for up to 2 hours.

44. The isolated mesenchymal stem cell of claim 35, wherein the mesenchymal stem cell is incubated for 1 hr with 1 μg/mL poly(LC).
